(19) United States
(12) Patent Application Publication
(10) Pub. No.: US 2014/0348867 A1
(43) Pub. Date: Nov. 27, 2014

(54) Isolated Brachyspira and Methods and Compositions for Expanding and Isolating Brachyspira

(71) Applicants: John Clare Samuel Harding, Humboldt (CA); Janet Elizabeth Hill, Saskatoon (CA); Joseph Rubin, Saskatoon (CA); Manuel Chirino-Trejo, Saskatoon (CA)

(72) Inventors: John Clare Samuel Harding, Humboldt (CA); Janet Elizabeth Hill, Saskatoon (CA); Joseph Rubin, Saskatoon (CA); Manuel Chirino-Trejo, Saskatoon (CA)

(21) Appl. No.: 14/233,414

(22) PCT Filed: Jul. 18, 2012
(86) PCT No.: PCT/CA2012/000682

§ 371 Date: Aug. 7, 2014

Related U.S. Application Data
(60) Provisional application No. 61/554,281, filed on Nov. 1, 2011.
(60) Provisional application No. 61/568,390, filed on Dec. 8, 2011.
(60) Provisional application No. 61/657,757, filed on Jun. 9, 2012.

(51) Int. Cl. C07K 14/20; C07K 16/12; C12N 1/20

(52) U.S. Cl. CPC C07K 14/20; C12N 1/20; C07K 16/1207
USPC 424/190.1; 435/252.1; 530/387.9

(57) ABSTRACT

The disclosure provides isolated—, compositions comprising—and methods of culturing —a Brachyspira sp. Sask30446 organism. The method comprises inoculating a liquid media or solid media with a sample comprising the Brachyspira sp. Sask30446 organism and incubating the liquid media or solid media at a temperature between 25-44° C. under anaerobic conditions. Also provided is isolated Brachyspira sp. Sask30446 organism, compositions comprising Brachyspira sp. Sask30446 organism and methods and uses thereof.

### RELATED APPLICATION

This application claims the benefit of priority of PCT Application PCT/CA2011/00828 filed Jul. 18, 2011 and provisional applications U.S. 61/554,281 filed Nov. 1, 2011; U.S. 61/568,390 filed Dec. 8, 2011 and U.S. 61/657,757 filed Jun. 9, 2012 each of which are herein incorporated by reference in their entirety.

### INCORPORATION OF SEQUENCE LISTING

A computer readable form of the Sequence Listing “Sequence Listing.txt” (47,697 bytes), created on Jul. 17, 2012, is herein incorporated by reference.

### FIELD OF THE DISCLOSURE

The present disclosure describes compositions and methods for culturing and isolating a Brachyspira species, such as Brachyspira sp. Sask30446, associated with a “dysentery-like” syndrome in swine. The disclosure provides isolated Brachyspira sp. Sask30446 and fragments or fractions thereof, immunogenic compositions comprising Brachyspira sp. Sask30446, and methods of inducing an immune response in a subject.

### INTRODUCTION

Recently a “dysentery-like” syndrome associated with high numbers of spirochetes has been identified that is unrelated to B. hyodysenteriae, B. pilosicoli, B. murdochii and B. intermedia). A novel species, Brachyspira sp. Sask30446 has been detected by PCR in the colon and other tissues of animals with the dysentery like syndrome.

Brachyspira species can be difficult to grow and the conditions that permit growth of a species are not necessarily applicable to other genera or to other species.

### SUMMARY OF THE DISCLOSURE

An aspect of the disclosure provides a method of culturing a Brachyspira sp. Sask30446 organism, the method comprising inoculating a liquid media or a solid media with a sample comprising the Brachyspira sp. Sask30446 organism and incubating the liquid media or solid media at a temperature between 25-44° C. under anaerobic conditions for example to provide an incubated culture.

In an embodiment, the solid media is an agar media selected from BAM, BJ, CVS, TSA, and Blood agar media. In another embodiment, the liquid media comprises Blood Heart Infusion (BHI) and/or Heart Infusion (HI) broth, 1-20% blood product and 0.5%-10% glucose.

In an embodiment, the method further comprises subculturing or passaging the Brachyspira sp. Sask30446 one or more passages.

In another embodiment, the subculturing comprises inoculating a liquid media or a further agar media with a portion of the agar media comprising a hemolytic zone comprising Brachyspira sp. Sask30446 organism.

Another embodiment includes a method of culturing a Brachyspira sp. Sask30446 organism, the method comprising:

a) inoculating BJ agar media, TSA media or CVS agar media with a sample comprising Brachyspira sp. Sask30446;

b) incubating the BJ agar media, TSA media or the CVS agar media at a temperature between 25-44° C., preferably at about 42° C. under anaerobic conditions for about 24 to 72 hrs, optionally about 40 to 56 hrs, preferably about 48 hrs or until zones of hemolysis are visible for example to provide an incubated culture; and

c) subculturing the zone of hemolysis.

In an embodiment, the anaerobic conditions comprise an atmospheric environment oxygen content of about 0% to about 2% oxygen.

In another embodiment, the sample comprising Brachyspira sp. Sask30446 is gastrointestinal tissue, gastrointestinal content and/or fecal material for example collected by fecal swab. In an embodiment, the sample cultured or used in a method herein is suspected of comprising Brachyspira sp. Sask30446.

In an embodiment, the temperature is between about 38-44° C. or between about 40-43° C., optionally about 42° C. for solid media and/or about 39° C. for liquid media.

In an embodiment, the subculturing comprises culturing Brachyspira sp. Sask30446 in a liquid media comprising BHI and/or HI, 1-20% blood product and 0.5%-10% glucose, preferably JBS media comprising BHI broth, about 5% ovine blood, about 5% fetal calf serum and about 1% glucose.

In another embodiment, the method comprises inoculating a liquid media with a zone of hemolysis comprising Brachyspira sp. Sask30446; incubating the inoculated liquid media under anaerobic conditions at a temperature between 25-44° C. to provide an incubated culture.

In yet another embodiment, the liquid media and/or the solid media comprise one or more antibiotics.

Also provided in another aspect is a method of a culturing a Brachyspira sp. Sask30446 organism from a sample from a subject infected or suspected of being infected with Brachyspira sp. Sask30446 comprising:

a) obtaining a sample from the subject;

b) inoculating a solid media with the sample and incubating at a temperature of 25-44° C. under anaerobic conditions; and

c) optionally one or more subculturing steps;

d) detecting the presence of a Brachyspira sp. Sask30446 polypeptide and/or polynucleotide in the sample and/or in the hemolytic zone and/or colony and/or incubated culture, which is indicative that the colony and/or hemolytic zone and/or incubated culture comprises Brachyspira sp. Sask30446.

Brachyspira sp. Sask30446 can for example be distinguished for example from Brachyspira pilosicoli and hyodysenteriae based on the phenotypic characteristics described herein for example in Tables 4 and/or 5.

Accordingly in an embodiment, wherein the method is for distinguishing Brachyspira sp. Sask30446 from known species such as Brachyspira pilosicoli and hyodysenteriae step d) is replaced and/or supplemented with determining the phenotypic characteristics of the incubated culture wherein detection of a strong hemolytic zone, ring phenomenon and one or more characteristics in Table 4 and/or 5 is indicative the that the colony and/or hemolytic zone and/or incubated culture comprises Brachyspira sp. Sask30446.

In an embodiment, the method comprises a subculturing step, the subculturing step comprising inoculating a liquid media or a solid media with a colony and/or hemolytic zone (e.g. agar section/slice comprising a hemolytic zone) and incubating the inoculated liquid media and/or solid media under anaerobic conditions at a temperature between 25-44° C. to provide an incubated culture.

In another embodiment, the liquid media comprises BHI and/or HI, 1-20% blood product and 0.5%-10% glucose and/or the solid media is selected from BAM, BJ, CVS and Blood agar media or a modified media thereof.

A further aspect includes a method of isolating a Brachyspira sp. Sask30446 organism from a sample comprising culturing a sample according to a method described herein and extracting/isolating the Brachyspira sp. Sask30446 organism from the liquid media or solid media.

In an embodiment, the Brachyspira sp. Sask30446 is extracted/isolated by separating the Brachyspira sp. Sask30446 from the liquid media.

In another embodiment, the isolated Brachyspira sp. Sask30446 is frozen.

In an embodiment, the liquid media and/or the solid media comprises one or more antibiotics.

Another aspect includes an isolated Brachyspira sp. Sask30446 organism, the organisim comprising one or more molecules having a sequence of SEQ ID NOs: 7, 8 9, 11, 12, 25-34, 37, and/or 42-45; or one or more molecules having a sequence with at least 92.3% sequence identity to any one of SEQ ID NOs: 7 to 9, 11 and 12; at least 97.5% sequence identity to SEQ ID NO: 25; at least 98.5% sequence identity to SEQ ID NO:26; at least 93.5% sequence identity to SEQ ID NO:27 or 31; at least 95.5% sequence identity to SEQ ID NO:28, 29 and/or 34; at least 99.5% sequence identity to SEQ ID NO: 30, 32 and/or 37; at least 92.5% sequence identity to SEQ ID NO:33; and/or any combination thereof.

In another embodiment, the isolated Brachyspira sp. Sask30446 organism comprises one or more molecules having a sequence of SEQ ID NO: 42, 43, 44, and/or 45, for example comprises a polypeptide comprising one or more sequences of SEQ ID NO: 42, 43, 44, and/or 45 and/or a nucleic acid molecule encoding one or more sequences of SEQ ID NO: 42, 43, 44, and/or 45; or one or more polypeptide or nucleic acid molecules encoding a polypeptide comprising a sequence having at least 92.5% sequence identity with SEQ ID NO: 42, 95.5% sequence identity with SEQ ID NO:43; at least 96.5% sequence identity with SEQ ID NO:44 and/or 94.5% sequence identity with SEQ ID NO: 45 and/or any combination thereof.

In an embodiment, Brachyspira sp. Sask30446 organisms share about 94.5% sequence identity, about 95% sequence identity, about 96% sequence identity, about 97% sequence identity, about 98% sequence identity, about 99% sequence identity or about 99.5% sequence identity in one or more sequences described herein (e.g. polypeptide and/or nucleic acid), for example over the full length of the sequence, or at least 100, at least 200, at least 300, at least 400 or at least 500 residues.

For example, the sequence identity can be nucleic acid sequence identity, amino acid sequence identity or identity between a nucleic acid sequence and a second nucleic acid sequence encoding a polypeptide described herein.

In an embodiment, the Brachyspira sp. Sask30446 comprises one or more molecules having a sequence of SEQ ID NOs: 7, 8 9, 11, 12, 25-34, 37, 42-45 and/or sequences with at least 94.5% at least 95%, at least 96%, at least 97%, at least 98%, at least 99%, or at least 99.5% sequence identity to SEQ ID NOs: 7, 8 9, 11, 12, 25-34, 37, 42, 43, 44, and 45.

In yet another embodiment, the Brachyspira sp. Sask30446 organism is packaged in a vial, such as a sterile vial and is optionally frozen, dessicated or refrigerated.

In an embodiment, the isolated Brachyspira sp. Sask30446 is a Gram negative, anaerobic, spirochete and beta-hemolysis positive bacteria.

In an embodiment, the isolated Brachyspira sp. Sask30446 causes a dysentery-like disease in swine.

In an embodiment, Brachyspira sp. Sask30446 is characterized by the bacteria strain deposited with the International Depository of Canada (IDAC) (1015 Arlington St., Suite H3390 Winnipeg, MB R3E 3R2) on Nov. 16, 2011 under Accession number 16111-01.

In a further embodiment, the isolated Brachyspira sp. Sask30446 is a gram-negative, anaerobic, spirochete bacteria characterized by the bacteria strain deposited with the International Depository of Canada (1015 Arlington St., Suite H3390 Winnipeg, MB R3E 3R2) on Nov. 16, 2011 under Accession number 16111-01.

In yet a further embodiment, the isolated Brachyspira sp. Sask30446 comprises genomic sequence with at least 94.5%, at least 95%, at least 96%, at least 97%, at least 98%, at least 99%, or at least 99.5% sequence identity to the bacteria strain deposited with the International Depository of Canada (IDAC) (1015 Arlington St., Suite H3390 Winnipeg, MB R3E 3R2) on Nov. 16, 2011 under Accession number 16111-01.

In an embodiment, the isolated Brachyspira sp. Sask30446 is attenuated, live or killed.

A further aspect includes a media composition comprising Brain Heart Infusion and/or Heart Infusion broth, 1 to 20% blood product and 0.5% to 10% glucose.

In an embodiment, the media composition comprises ovine blood and fetal calf serum.

In an embodiment, the media composition comprises 5% ovine blood product, 5% fetal calf serum and 1% glucose.

A further aspect includes a composition comprising an isolated Brachyspira sp. Sask30446 and optionally a carrier or diluent.

In an embodiment, the composition is a Brachyspira sp. Sask30446 organism and diluent and/or culture that is cultured according to a method described herein.

In an embodiment, the composition is an immunogenic composition. In an embodiment, the composition is a pharmaceutical composition and the carrier or diluent is a pharmaceutically acceptable carrier or diluent.

In an embodiment, the composition further comprises an adjuvant.

Also provided in another aspect is a use of the isolated Brachyspira sp. Sask30446 organism, a composition comprising the Brachyspira sp. Sask30446 organism and/or a composition comprising the isolated Brachyspira sp. Sask30446 organism and an isolated Brachyspira sp. Sask30446 polypeptide, selected from SEQ ID NO:11, 12, 26, 28, 30, 32, 34, and/or 42-45; a polypeptide having at least 92.3% sequence identity to any one of SEQ ID NOs: 11 and 12; a polypeptide having at least 98.5% sequence identity to SEQ ID NO: 26; a polypeptide having at least 95.5% sequence identity to SEQ ID NO: 28; a polypeptide having at least 99.5% sequence identity to SEQ ID NO: 30; a polypeptide having at least 99.5% sequence identity to SEQ ID NO: 32; a polypeptide having at least 95.5% sequence identity to SEQ ID NO: 34; a polypeptide having at least 92.5% identity with SEQ ID NO: 42, 95.5% sequence identity with SEQ ID NO:43; at least 96.5% identity with SEQ ID NO:44 and/or 94.5% identity with SEQ ID NO: 45 and/or any combination thereof; or a combination of two or more thereof, to induce an immune response in a subject.

A further aspect includes a method for inducing an immune response in a subject against the isolated Brachyspira sp. Sask30446 organism, a composition comprising the isolated Brachyspira sp. Sask30446 organism and/or a composition comprising isolated Brachyspira sp. Sask30446 organism and an isolated polypeptide of any one of SEQ ID NO:11, 12, 26, 28, 30, 32, 34, and/or 42-45; a polypeptide having at least 92.3% sequence identity to any one of SEQ ID NOs: 11 and 12; a polypeptide having at least 98.5% sequence identity to SEQ ID NO: 26; a polypeptide having at least 95.5% sequence identity to SEQ ID NO: 28; a polypeptide having at least 99.5% sequence identity to SEQ ID NO: 30; a polypeptide having at least 99.5% sequence identity to SEQ ID NO: 32; a polypeptide having at least 95.5% sequence identity to SEQ ID NO: 34 and/or or one or more polypeptides having at least 92.5% identity with SEQ ID NO: 42, 95.5% sequence identity with SEQ ID NO:43; at least 96.5% identity with SEQ ID NO:44 and/or 94.5% identity with SEQ ID NO: 45; or a combination of two or more thereof, comprising administering to the subject or a cell from the subject an effective amount of the isolated Brachyspira sp. Sask30446 organism or the isolated polypeptide.

In an embodiment the subject is a swine, preferably a pig.

In an embodiment, the sample is obtained from a swine, preferably a pig with hemorrhagic colitis.

Another aspect includes a process of obtaining a Brachyspira sp. Sask30446 organism comprising:

a) obtaining a sample from a subject infected or suspected of being infected with Brachyspira sp. Sask30446;

b) culturing the sample according to a method described herein to provide an incubated culture; and

c) optionally one or more subculturing steps;

d) determining the presence of a Brachyspira sp. Sask30446 polypeptide or polynucleotide in the sample and/or in the incubated culture; and

e) extracting/isolating Brachyspira sp. Sask30446 for example isolating a hemolytic zone and/or colony from solid media and/or isolating Brachyspira sp. Sask30446 from the incubated culture;

wherein the presence of a Brachyspira sp. Sask30446 polypeptide or polynucleotide in the sample and/or incubated culture and optionally the presence of a hemolytic (e.g. on horse blood), tiny, clear, wet/glistening, “fried egg” shaped colony or zone of hemolysis on the agar media indicates the extracted/isolated organism comprises Brachyspira sp. Sask30446.

Another aspect of the disclosure includes a kit comprising an isolated Brachyspira sp. Sask30446 organism, a composition comprising Brachyspira sp. Sask30446 and one or more components selected from resuspension diluent, vial and/or instructions for use.

In an embodiment, the kit is for use with a method or process described herein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be discussed in relation to the drawings in which:

FIG. 1. Growth of Brachyspira sp. Sask30446 on BAM-SR agar after 8 days at 37° C. (top). Colonies are <1 mm in diameter and have a “fried egg” shape. Haemolysis is apparent.

FIG. 2. Gram stain of colony from plate in FIG. 1. Lower panel shows detail of slide with 5.0 μm scale bar.

FIG. 3. Gram stain of colon contents from pig with hemorrhagic colitis showing abundant spirochetes (A) and detail of same slide (B). qPCR results using primers JH0224/JH0225 indicated ˜1×10⁷ Sask30446 organisms per gram of feces. FIG. 3C is electron micrographs of Brachyspira sp. Sask30446 recovered from broth culture.

FIG. 4 Hemolytic zones produced by Brachyspira sp. Sask30446.

FIG. 5. Image showing Ring phenomenon—a bright, enhanced zone of hemolysis produced around a hole in the agar where a plug is removed from a hemolytic zone. Note approximately the right 23 of the image is hemolytic; the ring is on the edge of the hemolytic zone.

FIG. 6. Dark field microscopy image of Brachyspira sp. Sask30446.

FIG. 7. Anaerobic jar containing broth cultures of B. sp. Sask30446 in JBS media.

FIG. 8. Phylogenetic tree based on alignment of 810 bp of the nox gene of Brachyspira spp., including B. sp. Sask30446. The alignment was created using CLUSTALw, followed by distance calculation (F84 matrix) and neighbour joining using PHYLIP. The tree is a consensus of 100 bootstrap iterations, and bootstrap values are indicated at the major nodes. GenBank accession numbers for nox sequences are indicated in the tree. Scale bar indicates 0.02 substitutions per site.

FIG. 9. Fecal consistency scores (line, left ordinate), fecal smear spirochete scores (grey bars, left ordinate) and B. sp. Sask30446 DNA concentration (triangle, right ordinate) for inoculated pigs. Pig IDs are indicated in the upper right corner of each panel. Fecal scores were done daily. Quantitative PCR was performed on colon tissue samples collected at necropsy. Fecal consistency scored as 0=normal, 1=wet cement, 2=runny, 3=mucoid, 4=bloody diarrhea. ND=fecal smear spirochete score not done.

FIG. 10 is a heat-map showing daily fecal scores and culture results for all experimental and control pigs.

FIG. 11 is a western blot and a Coomassie stained gel demonstrating antibody detection of Brachyspira sp. Sask30446 compositions.

### DETAILED DESCRIPTION

Brachyspira species including B. hyodysenteriae, B. pilosicoli, B. murdochii and B. intermedia are associated with intestinal disease including swine dysentery (B. hyo) and spirochetal colitis (B. pilo). Recently a “dysentery-like” syndrome associated with high numbers of spirochetes, but without recognized Brachyspira species or enteric pathogens has emerged. This novel spirochete, designated Brachyspira sp. Sask30446, is not detected and/or distinguished from other spirochetes using currently commercially available diagnostic tests.

Methods for identifying Brachyspira sp. Sask30446 using for example PCR are described in PCT/CA2011/000828 titled DIAGNOSTIC METHOD FOR COLITIS filed Jul. 18, 2011, which is herein incorporated by reference. An example of a method of identifying Brachyspira sp. Sask30446 is also described below. Other methods can also be used, for example using probes to detect Brachyspira sp. Sask30446 specific sequences, for example nox gene Brachyspira sp. Sask30446 specific sequences, as well as genus specific primers and sequencing to compare for example to sequences described herein.

Brachyspira species, including Brachyspira sp. Sask30446, can be difficult to culture. Described herein are methods for culturing and isolating Brachyspira sp. including Brachyspira sp. Sask30446. Also described is isolated Brachyspira sp. Sask30446, and compositions comprising Brachyspira sp. Sask30446. Compositions for growing Brachyspira species as well as processes of making immunogenic compositions comprising Brachyspira sp. Sask30446 are also provided.

Accordingly, an aspect of the disclosure includes a method of culturing a Brachyspira sp. Sask30446 organism comprising inoculating a liquid media or a solid media, the solid media or a liquid media comprising a blood product, with a sample comprising the Brachyspira sp. Sask30446 organism and incubating the liquid media or the solid media at a temperature between 25-44° C., preferably between 37° C. and 42° C. under anaerobic conditions, preferably of about 0% to about 2% oxygen, to provide an incubated culture (e.g. incubated solid culture or an incubated liquid culture, including for example primary culture or subculture). For example, an anaerobic gas pack can be used to reduce the oxygen concentration. In an embodiment, a commercially available anaerobic gas pack such as—Oxoid AnaeroGen™ is used, as directed. When used as directed Oxoid AnaeroGen™ can reduce oxygen levels in a vessel such a jar to below 1% within 30 minutes. In another embodiment, the anaerobic conditions are provided by an anaerobic chamber.

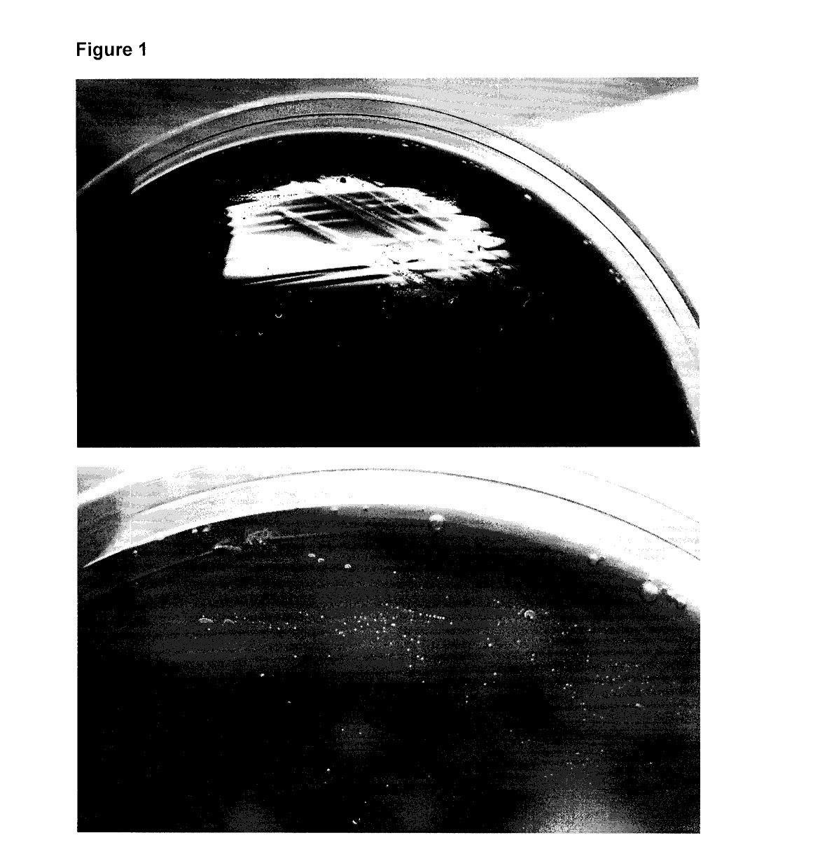

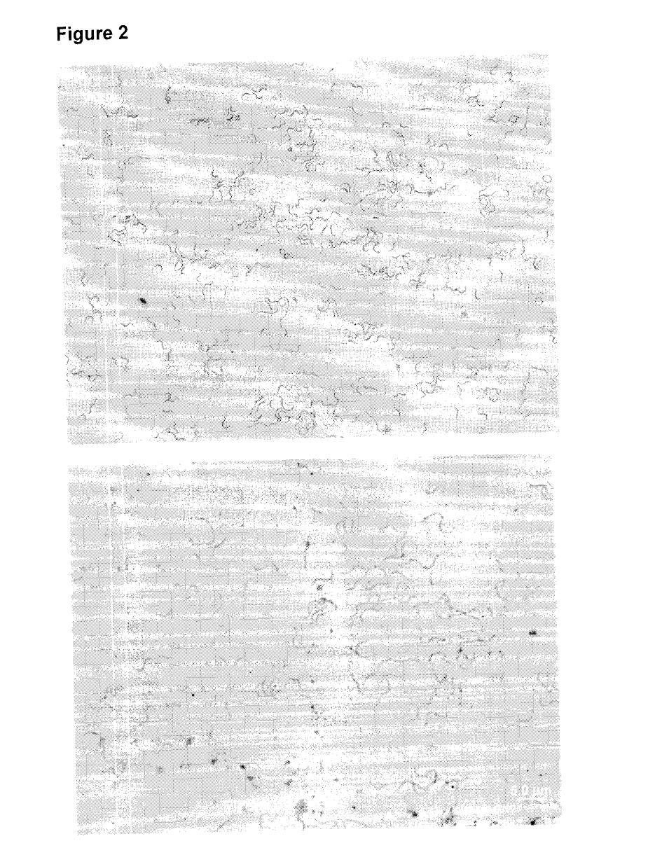

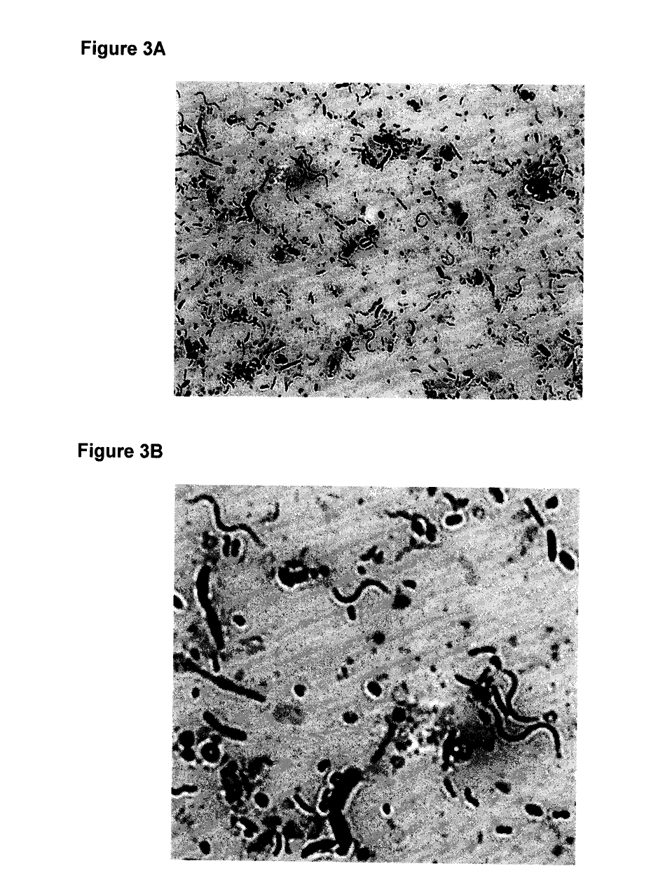

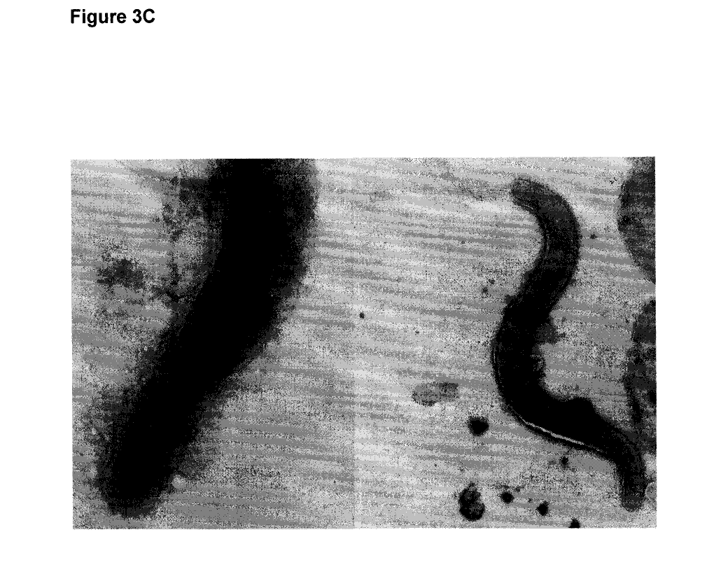

The term “Brachyspira sp. Sask30446” refers to a species of Brachyspira comprising one or more of SEQ ID NOs: 11, 12, 26, 28, 30, 32, 34, 42, 43, 44 and 45 and/or a sequence with at least 92.5, at least 93.5, at least 94.5 at least 95.5, at least 96.5, at least 97.5, at least 98.5, at least 99, at least 99.5%, at least 92.5%, at least 95.5%, at least 96.5%, at least 94.5% identity with a polynucleotide sequence encoding SEQ ID NOs: 11, 12, 26, 28, 30, 32, 34, 42, 43, 44 and/or 45 respectively and which are for example obtainable by a method disclosed herein (e.g. using a solid or liquid media described with methods provided herein). For example, NADPH oxidase polynucleotides from isolates of Brachyspira sp. Sask30446 share for example 99% sequence identity at the polynucleotide level (e.g. SEQ ID NOs: 7-9), whereas the closest known DNA relative shares only 92.2% sequence identity (SEQ ID NO:10). In an example, Brachyspira sp. Sask30446 isolates share at least 99% polypeptide sequence identity with at least one polypeptide having a sequence of SEQ ID NO: 11, 12, 26, 28, 30, 32, 42, 43, 44 and 45. Brachyspira sp. Sask30446 for example, when cultured on Blood Agar Media-SR (BAM-SR) agar supplemented with horse blood, under anaerobic conditions at for example 37-44° C., grow as hemolytic, small, clear, wet looking, “fried egg” shaped colonies (as in FIG. 1). Gram stain of isolates from agar plates show pleiomorphic, Gram negative spirochetes with tapered ends. Length of the cells is variable, for example about 2-20 μm, for example having an average of 8.5 μm (FIG. 2; Table 4). Brachyspira sp. Sask30446 can demonstrate hemolytic zones that are characterized by a ring phenomenon. Spirochetes with the same morphology as above are abundant in samples of colon contents, colon tissue and/or feces from pigs with hemorrhagic colitis that also test positive by the B. sp. Sask30446 qPCR assay (primers JH0224/JH0225) with counts of ˜1×10⁷ B. sp. Sask30446 organisms per gram of feces or colon contents (FIG. 3). In an embodiment, the number of copies of DNA is a proxy for organism number.

Brachyspira sp. Sask30466 is a laboratory identifier and the species may be renamed. A name under consideration is Brachyspira campestris and the species may be referred to as B. campestris herein. In recognition of terminology changes in the art, the strains described herein are phenotypically characterized, including by parameters of Gram stain, shape, type and hemolysis ability as well as other parameters described in Tables 4 and 5. Further, the strains are genotypically characterized by gene sequence for a number of genes. A person skilled in the art using for example a PCR based test described for example herein and/or in PCTCA2011000828, would be able to determine if an isolated Brachyspira sp. for example isolated and/or obtained using a method disclosed herein is a Brachyspira sp. Sask30446 organism. An isolate of Brachyspira sp. Sask30466 has been deposited with the International Depository of Canada (1015 Arlington St., Suite H3390 Winnipeg, MB R3E 3R2) on Nov. 16, 2011 under Accession number 16111-01. A “Brachyspira sp. Sask30446” can also therefore refer to a species having the same phenotypic and/or genotypic characteristics as the deposited Brachyspira sp. Sask30446 (Accession number 16111-01), and/or a species of Brachyspira comprising one or more polynucleotides having sequences in common with the deposited Brachyspira sp. Sask30446 (Accession number 16111-01) and/or with at least 92.5, 93, 93.5, 94, 94.5, 95, 95.5, 96, 96.5, 97, 97.5, 98, 98.5, 99 or 99.5% sequence identity with said polynucleotides.

In an embodiment, isolates that fall within the species share about 94.5, 95, 95.5, 96, 96.5, 97, 97.5, 98, 98.5, 99 or 99.5% pairwise nucleotide sequence identity.

Brachyspira sp. Sask30446 is a strain of what is believed to be a new species of Brachyspira based for example on NADH oxidase gene sequences and other sequences as described below. The name proposed for the species is Brachyspira campestris. Brachyspira campestris are phylogenetically distinct from recognized Brachyspira spp.

The term “anaerobic conditions” as used herein means conditions where the oxygen content of the atmospheric environment is about 4% oxygen or less, about 3% oxygen or less, about 2% oxygen or less, about 1% oxygen or less, about 0% oxygen, or alternatively about 0% to about 4%, about 0% to about 3%, about 0% to about 2% or about 0% to about 1% oxygen or about 0.5% to about 4%, about 0.5% to about 3%, about 0.5% to about 2% or about 0.5% to about 1% oxygen. The oxygen content can in another embodiment be about 0%, about 1%, about 2%, about 3% or about 4%.

In an embodiment, the sample is first determined or subsequently determined (e.g. subsequent to culturing or isolating) to comprise Brachyspira sp. Sask30446 using, for example the method described below or a method described in PCT/CA2011/000828 titled DIAGNOSTIC METHOD FOR COLITIS filed Jul. 18, 2011.

The “sample” can be any material that comprises (and/or is suspected to comprise) Brachyspira sp. Sask30446 organisms, such as but not limited to infected tissue, gastrointestinal content and/or fecal material for example collected by fecal swab obtained from a subject, such as a pig. The sample can be used directly as obtained from the source (e.g. animal such as pig) or following a pretreatment to modify the character of the sample. The sample can be or be derived from any biological gastrointestinal source such as tissues; cells, including primary cells (e.g. colon cells); as well as the contents of gastrointestinal tissues (e.g. cecal contents) and/or their end products such as feces (e.g. fecal swab). For example the sample can comprise contents, cells, and/or tissues derived from stomach, duodenum, ileum, colon, caecum and/or rectum. The sample can be for example a fresh tissue such as a biopsy, or a frozen source comprising the organism e.g. either frozen contents, frozen Brachyspira sp. Sask30446 culture stock (e.g. such as a frozen agar slice comprising a Brachyspira sp. Sask30446 hemolytic zone or liquid culture) or frozen infected tissue. The sample can be preprocessed or treated prior to use (e.g. pretreated), such as diluting viscous fluids, and the like. Methods of pre-treatment can involve fractionation, filtration, distillation, concentration, inactivation of interfering components, the addition of reagents, and the like.

The sample to be cultured can for example be a part of the same sample confirmed to comprise Brachyspira sp. Sask30446, a pretreated or processed sample derived from this sample or a sample obtained from the same source (e.g. a fecal swab from an animal confirmed to be positive for Brachyspira sp. Sask30446). The sample can also for example be a sample suspected of comprising Brachyspira sp. Sask30446 based for example on animal disease symptoms and/or the lack of other pathogenic Brachyspira species. Growth characteristics and sequence confirmation can be subsequently used to determine if the Brachyspira species is Brachyspira sp. Sask30446

Brachyspira species can live in the colonic crypts. Accordingly, in an embodiment, the sample is fresh tissue (such as colon, cecum or rectum). In another embodiment, the sample is a rectal swab (e.g. obtained by contacting mucosa ˜5 cm inside rectum).

In an embodiment, the sample is obtained from a swine. In an embodiment, the swine is a pig.

As a person skilled in the art would know, samples obtained from a subject should be placed in sealed containers to avoid cross contamination.

In an embodiment, samples such as fresh tissue or gastrointestinal content (e.g. fecal swab) are maintained at about 4° C., for example in a refrigerator. In another embodiment, the samples are stored frozen, for example at about −20° C. or about −80° C.

The term “colon” as used herein means the large intestine, for example the large intestine of a subject pig, and “colon contents” means digested material contained within the colon including the rectum.

The term “colonic tissue” as used herein means tissue derived from the colon.

The term “colon cell” as used herein refers to a cell of colonic tissue.

The term “caecum” or “cecum” as used herein means the first portion of the large intestine that forms an elongated dilated pouch in the pig.

The term “fecal material” as used herein refers to digestive waste products that have been expelled or could be expelled from the subject during defecation.

As Brachyspira spp. are delicate, multiple freeze thaw cycles are preferably avoided.

The media is for example contained in a vessel such as a plate (e.g. petri dish), tube (e.g. agar slant) or flask (e.g. liquid media). Inoculating a liquid media or a solid media means for example inoculating solid media where the solid media is comprised in such a vessel.

In an embodiment, the solid media is an agar media. The agar media can be for example comprised in an agar plate, an agar slant or any other tissue culture appropriate vessel comprising agar media.

In an embodiment, the agar media (e.g. also referred to as “agar”, “agar plate”, etc.) is inoculated by smearing the sample on a portion of the agar media (e.g. agar plate). In an embodiment, the agar media can be inoculated by touching a loop to a tissue lesion comprising Brachyspira sp. Sask30446 and transferring a small amount of material to the agar media. Although broth enrichment or filtering of the sample is not required to grow Brachyspira sp. Sask30446 on solid media, in an embodiment, either or both of these steps are taken prior to inoculating the agar media.

In an embodiment, the media e.g. solid agar or liquid media comprises at least one blood product. In an embodiment, the inoculum is an isolated Brachyspira sp. Sask30446 or a pure culture, where the inoculum is an isolated colony or pure culture, antibiotics can be omitted from the media. In an embodiment, where the inoculum is not an isolated Brachyspira sp. Sask30446 or pure culture, e.g. a tissue sample, the media comprises at least one antibiotic.

In an embodiment, the media comprises a sugar, for example glucose.

In an embodiment, the agar media is selected from BAM media (see for example Vet Microbiol. 2005 Feb. 25; 105(3-4):229-34. Epub 2004 December 28. Rapid isolation of Brachyspira hyodysenteriae and Brachyspira pilosicoli from pigs. Calderaro A, Bommezzadri S, Piccolo G, Zuelli C, Dettori G, Chezzi C.), BJ media (see for example Improved selective medium for the isolation of Treponema hyodysenteriae. R A Kunkle and J M Kinyon J Clin Microbiol. 1988 November; 26(11): 2357-2360), and CVS media (Colisitin, Vancomycin, Spectinomycin; see for example Vet Microbiol. 2005 Feb. 25; 105(3-4):229-34. Epub 2004 Dec. 28. Rapid isolation of Brachyspira hyodysenteriae and Brachyspira pilosicoli from pigs. Calderaro A, Bommezzadri S, Piccolo G, Zuelli C, Dettori G, Chezzi C), TSA media (trypticase soy agar with 5% bovine blood) and Blood agar (Trypticase Soy Agar with 5% sheep's blood) and/or a modified version thereof, for example where any one or more of, or each of, the components is varied for example by up to about 50%, about 40%, about 30%, about 20% or about 10%. The Blood Agar can be purchased for example from Becton Dickinson, in Sparks, Md. Recipes for BJ, CVS and BAM are known in the art, and exemplary recipes for BJ, CVS and BAM are provided below. For example, BAM is a blood agar media comprising Blood agar base 2, beef extract, and peptone. BAM-SR additionally comprises antibiotics spectinomycin and rifampin. TSA media comprises enzymatic digests of casein and soybean meal supplemented with blood for example comprising tryptone, soytone sodium chloride and agar plus for example 5% bovine blood.

In an embodiment, the media is a modified version of BAM, TSA, BJ media, CVS or Blood agar e.g. modified BJ media, modified CVS media etc. In an embodiment, the media is a modified BJ media, a modified CVS media, a modified BAM media, wherein the amounts of one or more of the components is varied by up to about 50%, about 40%, about 30%, about 20% or about 10%. The modified media can include for example additional antibiotics and/or additional components.

In an embodiment, the media comprises at least one antibiotic selected from Colistin, Vancomycin, Spiramycin, Rifampin and Spectinomycin. In an embodiment, a combination of antibiotics is used, for example, Rifampin and Spectinomycin, or Colistin, Vancomycin and Spiramycin. Other combinations include for example Colistin, Vancomycin, Spiramycin, Rifampin and Spectinomycin. Antibiotics are added and particularly important when culturing from a sample obtained from a subject, such as fecal sample comprising a number of organisms.

In an embodiment, the antibiotic is selected from a rifamycin, glycopeptide, aminocyclitol, macrolide and/or polymyxin antibiotic.

In an embodiment, the concentration of Colistin can be about 3.125 μg/ml about 12.5 μg/ml (e.g. 6.25 μg/ml); the concentration of Vancomycin can be about 3.125 μg/ml about 12.5 μg/ml (e.g. 6.25 μg/ml); the concentration of Spiramycin can be about 12 μg/ml about 50 μg/ml (e.g. 25 μg/ml); the concentration of Rifampicin can be about 6.25 μg/ml-about 25 μg/ml (e.g. 12.5 μg/ml); the concentration of Spectinomycin can be about 50 μg/ml about 400 μg/ml (e.g. 200 μg/ml).

In an embodiment, the concentration of the antibiotic for example Colistin is about 3.125 μg/ml, about 6.25 μg/ml or about 12.5 μg/ml. In an embodiment, the concentration of the antibiotic, for example Vancomycin, is about 3.125 μg/ml, about 6.25 μg/ml or about 12.5 μg/ml. In another embodiment, the concentration of the antibiotic for example Spiramycin, is about 12 μg/ml, 25 μg/ml, or about 50 μg/ml. In another embodiment, the concentration of the antibiotic, for example Rifampicin, is about 6.25 μg/ml, about 12.5 μg/ml or about 25 μg/ml. In another embodiment, the concentration of the antibiotic is about 6.25 mg/ml, about 12.5 μgml or about 30 μg/ml. In another embodiment, the concentration of antibiotic, for example Spectinomycin, is about 50 μg/ml, about 200 μg/ml, or about 400 μg/ml. The antibiotic can also be for example at any amount between the stated ranges, for example in increments of about 5 μg/ml, about 10 μg/ml or about 20 μg/ml.

In an embodiment, the method of culturing a Brachyspira sp. Sask30446 organism comprises inoculating a blood agar media (BAM e.g. BAM-SR) or a modified BAM and incubating the inoculated media at a temperature between 25-44° C. under anaerobic conditions.

In an embodiment, the media, for example BAM, comprises a blood product.

In an embodiment, the blood is mammalian blood. In an embodiment, the blood is horse blood. In another embodiment the blood is ovine (sheep) blood. In yet a further embodiment, the blood is bovine blood. In a further embodiment, the blood is defibrinated blood. In an embodiment, the blood is whole blood.

In an embodiment, the media, for example BAM, comprises 5-10% mammalian blood. In an embodiment, the media, for example BAM, comprises about 5%, about 6%, about 7%, about 8%, about 9% or about 10% blood.

In an embodiment, the BAM comprises blood agar base and a mammalian blood product. In an embodiment, the blood agar base is blood agar base no. 2 (Oxoid). For example, the composition of blood agar base No. 2 comprises 15 g/L proteose peptone, 2.5 g/L liver digest, 5.0 g/L yeast extract, 5.0 g/L sodium chloride, 12 g/L agar. These are all generic products that could be purchased from any chemicalmedia supplier for example Difco or Becton Dickinson (BD) (Sparks Md.).

In an embodiment, the media is a modified BAM media wherein the amount of one or more or each of the components can be varied by about 50%, about 40%, about 30%, about 20% or about 10%.

In an embodiment, the blood agar base is 20 to 60 g/L for example 30, 35, 40, 45 or 50 g/L of the BAM. In an embodiment, the BAM comprises beef extract. In an embodiment, the beef extract is about 1.5 g/L, about 2 g/L, about 2.5 g/L, about 3 g/L, about 3.5 g/L, about 4 g/L, or about 4.5 g/L. In a further embodiment the BAM comprises Bacto Peptone (Difco) optionally at about 2.5 g/L, about 3 g/L, about 3.5 g/L, about 4 g/L, about 4.5 g/L, about 5 g/L, about 5.5. g/L, about 6 g/L, about 6.5 g/L about 7 g/L or about 7.5 g/L. In an embodiment, the BAM comprises antibiotics, such as spectomycin and/or rifampin. Other antibiotics can optionally be included.

In an embodiment, the BAM comprises Blood Agar Base no. 2 (Oxoid) (40 g/L), Beef Extract (Difco) (3 g/L) and Bacto Peptone (Difco) (5 g/L)), supplemented with defibrinated horse blood (7%), spectinomycin (400 μg/ml) and rifampin (15-30 μg/ml) (e.g. BAM-SR) (Calderaro et al., 2005).

In an embodiment, the solid media is inoculated directly for example with a sample of an infected subject, for example a sample of colon tissue from an infected pig. In an embodiment, the sample is incubated in anaerobic liquid media and the liquid media is used to inoculate the agar culture. In another embodiment, the inoculation comprises placing a filter comprising sample on solid media such as BAM, for example filtering the sample (e.g. colon tissue in anaerobic media) through a 0.45 μm filter such that the sample is retained on the filter and placing the filter on the BAM. For example, the tissue is placed on top of a filter so that the organisms have to swim through the filter to reach the agar media. Non-motile organisms and other debris are retained on the filter. In another embodiment, the method comprises inoculation of media with broth, e.g. anaerobic broth that was incubated with the tissue sample, for example at room temperature for 30 minutes, through a filter. For example, 2 drops of broth are dripped onto a filter placed on agar media and incubated.

In an embodiment, the agar media, for example BAM, is incubated for at least 1 day, at least 2 days, at least 4 days, at least 6 days, at least 8 days or at least 10 days. For example, after 8 days of anaerobic incubation using BAM-SR agar Brachyspira sp. Sask30446 is visible as a hemolytic (on horse blood), tiny, clear, wet/glistening, “fried egg” shaped colony. The colonies are less than 1 mm in diameter (FIG. 1).

In an embodiment, the method comprising culturing a Brachyspira sp. Sask30446 organism comprises inoculating a BJ agar media or CVS agar media or modified BJ media or modified CVS media and incubating the BJ agar media or the CVS agar media (or modified media) at a temperature between 25-44° C., preferably 37-44° C. under anaerobic conditions. In an embodiment, the agar media is BJ. In another embodiment, the media is CVS. In another embodiment, the media is modified BJ or modified CVS.

BJ and CVS media have been shown to have useful properties. For example, these media have been found when culturing Brachyspira sp. Sask30446, to be useful for one or more of: inhibiting growth of contaminating organisms, increasing quantity of growth (e.g. proportion of the plate with hemolytic zones), intensity of hemolysis and the discernibility of the “ring” phenomenon. The “ring” phenomenon refers to production of a bright, enhanced zone of hemolysis produced around a hole in the agar where a plug is removed from a Brachyspira sp. Sask30446 hemolytic zone (see FIG. 5). Growth of Brachyspira sp. Sask30446 is indicated for example on BJ and CVS agar media by the appearance of hemolytic zones. A hemolytic zone can be subcultured one or more times allowing for example multiple rounds of purification.

As used herein “BJ media” or “BJ agar media” means an agar media comprising at least Trypticase soy agar, Pig Feces Extract, Colistin, Vancomycin, Spiramycin, Rifampicin, Spectinomycin and a blood product optionally a bovine blood product, at the concentrations as specified elsewhere herein or known in the art. A recipe for BJ media includes Trypticase soy agar 40 g; Pig Feces Extract 50 ml, Sterile distilled water 976 ml, Colistin—3.125 μg/ml-12.5 μg/ml (e.g. 6.25 μg/ml) concentration in final plate; Vancomycin—3.125 μg/ml-12.5 μg/ml (e.g. 6.25 μg/ml) concentration in final plate; Spiramycin—12 μg/ml-50 μg/ml (e.g. 25 μg/ml) concentration in final plate; Rifampicin—6.25 μg/ml-25 μg/ml (e.g. 12.5 μg/ml) concentration in final plate Spectinomycin—50 μg/ml-400 μg/ml (e.g. 200 μg/ml) concentration in final plate and Bovine blood 50 ml.

As used herein “modified BJ media” as used herein means BJ media where one or more of the components is decreased or increased by up to about 10%, 20%, 30%, 40% or 50% for example, modified BJ media can include Trypticase soy agar 30-50 g; Pig Feces Extract 30-70 ml; Sterile distilled water; Colistin—3.125 μg/ml-12.5 μg/ml (e.g. 6.25 μg/ml) concentration in final plate; Vancomycin—3.125 μg/ml-12.5 μg/ml (e.g. 6.25 μg/ml) concentration in final plate; Spiramycin—12 μg/ml-50 μg/ml (e.g. 25 μg/ml) concentration in final plate; Rifampicin—6.25 μg/ml-25 μg/ml (e.g. 12.5 μg/ml) concentration in final plate; Spectinomycin—50 μg/ml-400 μg/ml (e.g. 200 μg/ml) concentration in final plate; and Bovine blood 40-60 ml. The modified media can also contain one or more additional antibiotics or one or more additional components.

As used herein “CVS media” or “CVS agar media” means an agar media comprising at least Trypticase soy agar, Colistin, Vancomycin, Spectinomycin and a blood product, such as bovine blood. The concentrations of components can be as described elsewhere.

As used herein “modified CVS media” as used herein means CVS media where one or more of the components is decreased or increased by up to about 10%, 20%, 30%, 40% or 50% for example. The modified media can also contain one or more additional antibiotics or one or more additional components.

As used herein “JBS media” means a broth media comprising at least brain heart infusion (BHI), BHI+about 1% glucose (wt/vol) with about 5% ovine blood (vol/vol), and about 5% calf serum (vol/vol), such as deactivated fetal calf serum.

It has been found that Brachyspira sp. Sask30446 colonies and/or zones of hemolysis grown on solid agar can be expanded using JBS media, e.g. comprising BHI+1% glucose with 5% blood and 5% serum. In some embodiments, colonies and/or zone of hemolysis are transferred to JBS to initiate a starter culture, for example the colony or zone of hemolysis is transferred to about 2 mL, 3 mL, 4 mL, 5 mL, 6 mL, 7 mL, 8 mL, 9 mL, 10 mL, 15 mL or more of JBS broth and cultured for a suitable time. In certain embodiments, the starter culture is then transferred to a larger volume of liquid broth. The liquid broth can for example be JBS or other media such media comprising HI and serum.

Media comprising HI and serum can for example include about 5% serum, 6% serum, 7% serum, 8% serum, 9% serum, 10% serum, or more e.g. 20% serum.

In an embodiment, the method comprises: a) inoculating BJ agar media or CVS agar media or a modified form thereof with a sample comprising Brachyspira sp. Sask30446; b) incubating the BJ agar media or the CVS agar media or modified form thereof at a temperature between 25-44° C. under anaerobic conditions for about 24 to 72 hrs, optionally about 40 to 56 hrs, preferably about 48 hrs or until zones of hemolysis are visible; and c) optionally subculturing the zones of hemolysis.

For example, after inoculating the BJ or CVS agar media or modified form thereof, the agar media (e.g. plates) is placed in an anaerobic environment such as an anaerobic chamber or jar with for example Oxoid anaerobic gas packs. Very good growth is achieved for example after about 48 hours where growth is indicated by zones of hemolysis. At about 48 hours for example, the anaerobic jar is opened, the plate or other vessel removed and a Brachyspira sp. Sask30446 hemolytic zone is sub-cultured to fresh media. Multiple subcultures (for example 2, 3, 4 or more) can be used for example to obtain a pure culture. Subculturing is also used for example to propagate the culture. For each sub-culture, typically a single round zone of hemolysis is subcultured on to fresh media and incubated as previously described. Subculturing involves for example contacting the Brachyspira sp. Sask30446 hemolytic zone with a sterile inoculating instrument or obtaining a portion of agar media from a Brachyspira sp. Sask30446 hemolytic zone and inoculating an agar media (e.g. by streaking the agar across the agar media using sterile technique with a sterile loop) with the Brachyspira sp. Sask30446 hemolytic zone and/or inoculating a liquid media with Brachyspira sp. Sask30446.

The hemolytic zones produced by Brachyspira sp. Sask30446 on for example BJ and/or CVS agar media can be considered “Strong 3-hemolysis” similar to for example B. hyodysenteriae and in contrast to B. pilosicoli which produces “Weak β-hemolysis”.

Brachyspira sp. Sask30446 grown for example on BJ or CVS agar media is also “ring” positive. For example, when a plug of agar from a hemolytic zone is removed and the plate is re-incubated, a bright, enhanced ring of hemolysis surrounds the hole in the media. See for example FIG. 5.

In an embodiment, the incubation temperature is between about 35-44° C. (e.g. 35° C., 36° C., 37° C., 38° C., 39° C., 40° C., 41° C., 42° C., 43° C., 44° C., or any 0.1° C. increment between 35° C. and 44.9° C.), between about 38-44° C., between 40-43° C. or between 39-42 C. In another embodiment, the temperature is about 35° C., about 36° C., about 37° C. or about 38 C. In an embodiment, the temperature is about 39° C., about 40° C., about 41° C., about 42° C., about 43° C. or about 44° C. In an embodiment, the temperature is about 42° C. For example, solid media cultures are incubated at about 42° C. and liquid media cultures are inoculated at about 39° C.

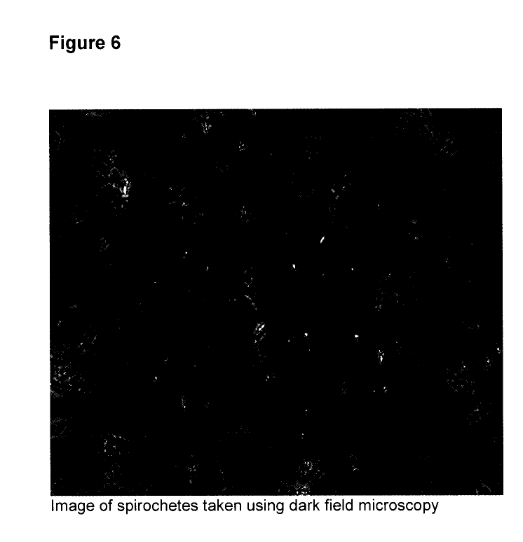

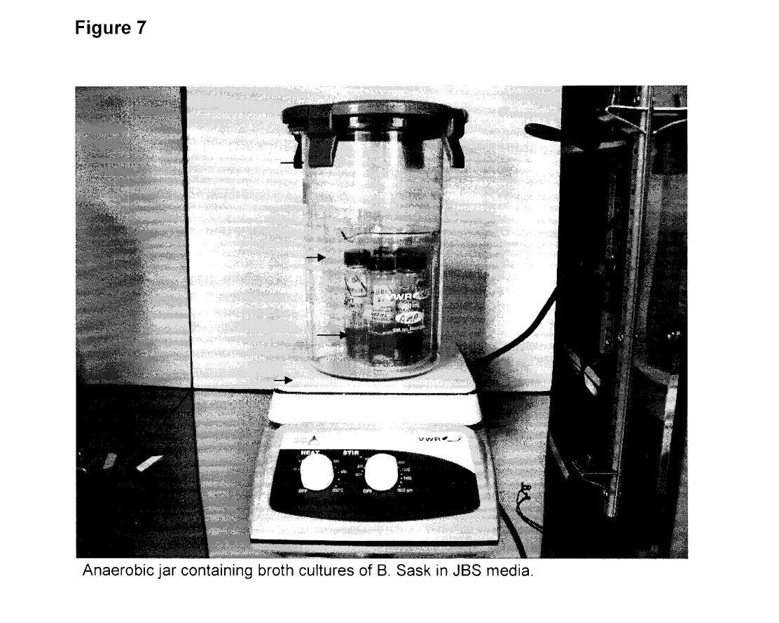

In an embodiment, the cultured Brachyspira sp. Sask30446 is Gram stained. Gram stains of isolates from agar plates show pleiomorphic, Gram negative spirochetes with tapered ends. Length of the cells is variable, 2-20 μm (FIG. 2, Table 4). Spirochetes with the same morphology are abundant in samples of colon contents (feces) from pigs with hemorrhagic colitis that also test positive by the Brachyspira sp. Sask30446 qPCR assay (primers JH0224/JH0225) with counts of ˜1×10⁷ Brachyspira sp. Sask30446 organisms per gram of feces or colon contents (FIG. 3). Brachyspira sp. Sask30446 can be visualized for example using dark field microscopy (FIG. 6) or hanging drop microscopy.

Another embodiment includes a method of a culturing a Brachyspira sp. Sask30446 organism from a sample from a subject infected or suspected of being infected with Brachyspira sp. Sask30446 comprising:

a) obtaining a sample from the subject;

b) inoculating a liquid media or a solid media with the sample and incubating at a temperature of 25-44° C. preferably 37-44° C. under anaerobic conditions to obtain a colony and/or hemolytic zone; and

c) optionally one or more subculturing steps;

wherein the presence of a Brachyspira sp. Sask30446 polypeptide and/or polynucleotide in the sample and/or in the hemolytic zone and/or colony and/or one or more of the phenotypic characteristics described in Tables 4 and/or 5 is indicative that the colony and/or hemolytic zone is Brachyspira sp. Sask30446.

In an embodiment, the presence of hemolytic (e.g. on horse blood), tiny, clear, wet/glistening, “fried egg” colonies and/or the presence of zones of hemolysis indicates growth of Brachyspira sp. Sask30446 is also indicative that the colony and/or hemolytic zone is Brachyspira sp. Sask30446.

It is further disclosed herein that Brachyspira sp. Sask30446 can be propagated using liquid media. Brachyspira sp. Sask30446 could not be cultured or cultured consistently in standard liquid broths (e.g. Brain Heart Infusion and 10% deactivated calf serum) under standard conditions e.g. at 39° C. or 42° C. Accordingly, an aspect the disclosure provides a method of propagating a Brachyspira sp., such as Brachyspira sp. Sask30446 comprising inoculating a liquid media comprising 1-20% blood product and/or 0 to 10% glucose, incubating the inoculated liquid media at 25-44° C. preferably 37-44° C. under anaerobic conditions to provide an incubated liquid culture; and optionally passaging the incubated liquid culture one or more times.

In an embodiment, the media is incubated in an environment with a minimum and/or maximum level of CO₂. In an embodiment, the maximum atmospheric CO₂ level for solid media is less than or equal to 20%, less than or equal to 15% or less than or equal to 13%. In an embodiment, the minimum level is for example 5%, 10% or 13%.

The liquid media can for example be a liquid media that is suitable for Brachyspira sp. comprising Brain Heart Infusion (BHI), Heart Infusion (HI) broth or the like, 1-20% blood product and 0.5%-10% glucose. In an embodiment, the liquid media comprises brain heart infusion (BHI) broth with 0.5% to 10% glucose and 0-20% of a blood product, for example whole blood and/or whole blood in combination with serum. In an embodiment, the liquid media is JBS, described below. In another embodiment, the liquid media comprises trypticase soy broth (TSB).

BHI broth (available as a powder for reconstitution from Becton Dickinson, Sparks, Md.) is a general purpose growth media that supports the growth of a wide variety of organisms, made from boiled hearts and brains, for example cattle hearts and brain. The broth can be powdered for later reconstitution, for example with water.

Similarly, HI broth was made by reconstituting HI powder which can be obtained for example from Oxoid limited, (Basingstoke, United Kingdom).

In an embodiment, the blood product is an ovine blood product. In another embodiment, the blood product is selected from a calf blood product or other suitable source of blood product. As used herein “blood product” means whole blood or a fraction such as serum. The blood product can be defibrinated and/or can be inactivated for example by heat inactivation, and/or can be filtered.

In an embodiment, the blood product is ovine blood product. In an embodiment, the liquid media comprises e.g. by volume at least 1%, 2%, 3%, 4%, 5%, 6%, 7%, 8%, 9%, or at least 10% blood product optionally an ovine blood product. In an embodiment, the liquid media comprises at most 20%, 19%, 18%, 17%, 16%, 15%, 14%, 13%, 12%, 11% or at most 10% blood product.

In another embodiment, the liquid media further comprises at least 1%, 2%, 3%, 4%, 5%, 6%, 7%, 8%, 9%, or at least 10% calf blood product (e.g. by volume). In an embodiment, the liquid media comprises at most 20%, 19%, 18%, 17%, 16%, 15%, 14%, 13%, 12%, 11% or at most 10% calf blood product.

In an embodiment, the blood product (e.g. ovine and/or calf) is whole blood, or a fraction thereof such as serum, plasma and the like. In an embodiment, the blood product (e.g. ovine and/or calf) is a fetal blood product, such as a deactivated fetal blood product.

In an embodiment, the liquid media comprises at least 0.5%, 1%, 2%, 3%, 4%, 5%, 6%, 7%, 8%, 9% or at least 10% glucose (weight/volume). In an embodiment, the liquid media comprises at least 1% glucose.

In an embodiment, the liquid media comprises brain heart infusion (BHI) broth, such as at least 80%, 81%, 82%, 83%, 84%, 85%, 86%, 87%, 88%, 89%, 90%, 91%, 92%, 93%, 94%, 95% by volume. In a further embodiment, the liquid media comprises brain heart infusion broth, glucose, ovine blood product and calf blood product. In an embodiment, the liquid media comprises about 90% BHI broth, about 1% glucose (wt/vol), about 5% ovine blood product (vol/vol) and about 5% calf blood product (vol/vol).

In an embodiment, the anaerobic conditions comprise about 4% oxygen or less, about 3% oxygen or less, about 2% oxygen or less about 1% oxygen or less about 0% oxygen, or alternatively about 0% to about 4%, about 0% to about 3%, about 0% to about 2% or about 0% to about 1% oxygen, about 0.5% to about 4%, about 0.5% to about 3%, about 0.5% to about 2% or about 0.5% to about 1% oxygen. The oxygen content can in another embodiment be about 0%, about 0.5%, about 1%, about 2%, about 3% or about 4%.

In an embodiment, the liquid media is incubated for example at 35-44° C., for example at 37-42° C., or at any 0.1° C. increment between 35° C. and 41.9° C., between about 38-42° C., or between 39-42° C. In another embodiment, the minimum temperature is about 35° C., about 36° C., about 37° C., or about 38° C. In an embodiment, the maximum temperature is about 39° C., about 40° C., about 41° C., or about 42° C. In an embodiment, the temperature is about 39° C. In an embodiment, the liquid media is incubated at about 35° C., about 36° C., about 37° C., about 38 C about 39° C., about 40° C., about 41° C. or about 42° C.

In an embodiment, the incubated culture is incubated for a sufficient time, about 24 hrs, about 48 hrs, about 72 hrs, or until a desired level of growth is achieved. A person skilled in the art would be familiar with principles for assessing suitable lengths of incubation to obtain a desired outcome (e.g. size of hemolytic zone) or level of growth (e.g. number of spirochetes). In an embodiment, an inoculated liquid media is incubated for a period of time sufficient to permit logarithmic growth and is harvested for example during the logarithmic growth phase.

In an embodiment, the media comprises antibiotics or other commonly used reagents for tissue culture growth of an organism as would be known to a person skilled in the art. For example, in applications where a liquid media is being inoculated directly, antibiotics such as those described elsewhere would be added to the liquid media to prevent growth of non Brachyspira sp. Sask30446 organisms. Where the liquid culture is inoculated with a purified Brachyspira sp. Sask30446 colony or hemolytic zone, antibiotics in the liquid media can be reduced and/or omitted.

In an embodiment, the Brachyspira sp. is Brachyspira sp. Sask30446. It is predicted that other Brachyspira species that share for example high homology with Brachyspira sp. Sask30446 can be propagated using the present methods and compositions.

In another aspect, the disclosure provides use of a media disclosed herein for culturing or propagating Brachyspira sp. Sask30446.

The methods and compositions permit culturing propagation and isolation of Brachyspira sp. Sask30446 when the inoculum (e.g. sample) comprises Brachyspira sp. Sask30446. The inoculum can be any source comprising the Brachyspira sp. For example, the inoculum can be for example, a section of solid media comprising Brachyspira sp. Sask30446, a sample comprising Brachyspira sp. Sask30446 such an infected tissue, fecal swab or intestinal content. The inoculum is in an embodiment, derived from a subject sample (e.g. a solid media section comprising Brachyspira sp. grown and/or isolated from a sample obtained from a subject). The inoculum can also for example be a liquid culture comprising Brachyspira sp. Sask30446. Brachyspira sp. Sask30446 can be propagated for example by subculturing, and/or passaging an incubated liquid culture comprising Brachyspira sp. Sask30446. Accordingly, in an embodiment, the method further comprises one or more passaging or subculturing steps. A person skilled in the art would be familiar with passaging cultures (liquid and solid) and using for example, sterile technique.

The methods described herein can also be suitably applied to related Brachyspira spp., for example phlyogentically closely related and/or phenotypically closely related. In an embodiment, the related Brachyspira spp. exhibits at least 80%, at least 85%, at least 90% sequence or at least 93%; at least 94%; at least 95% sequence identity to 2 or more, such as 3 or more, 4 or more or 5 or more Brachyspira sp. Sask30446 nucleic acid molecules or polypeptides disclosed herein.

The term “isolated” as used herein for example with respect to Brachyspira sp. Sask30446 means that the microorganism or fragment thereof, has been increased in purity, wherein “purity” is a relative term, not “absolute purity.” In particular aspects, an isolated or a purified Brachyspira sp. Sask30446 is 60% free, preferably at least 75% free, and more preferably at least 90% free from other components with which it is naturally associated or associated following culturing.

A further aspect includes a method of isolating a Brachyspira sp. Sask30446 organism from a sample comprising culturing/propagating a sample according to the method described herein, for example using solid media and/or liquid media; and extracting/isolating the Brachyspira sp. Sask30446 organism from the solid or liquid media.

The extracting or isolating step can comprise when from a solid media, removing a colony or cutting a portion of the solid agar comprising a hemolytic zone and optionally removing all or part of the solid media, for example slicing a top layer of agar from the solid media and/or dissolving the media. When extracting from liquid media, the isolating step can comprise centrifuging the inoculated incubated liquid media for example, according to a method known in the art, and removing the growth media. Alternatively, the liquid media can be removed by aspirating and/or decanting.

For example, in an embodiment, a solid media for example BJ, Blood agar or CVS is inoculated with a sample comprising Brachyspira sp. such as Brachyspira sp. Sask30466 (or suspected to comprise the organism), the solid media is incubated under anaerobic conditions for about 24-72 hours and a solid media section comprising a hemolytic zone is used to inoculate a liquid media comprising 1-20% blood product and glucose; the liquid media is incubated for 12-48 hrs, for example with rotation; the inoculated incubated liquid media is optionally sub-cultured and the organism harvested.

Another aspect includes a process of obtaining a Brachyspira sp. Sask30446 organism comprising:

a) obtaining a sample from a subject infected or suspected of being infected with Brachyspira sp. Sask30446;

b) culturing the sample according to a method described herein, e.g. inoculating a solid media with the sample and incubating at a temperature of 25-44° C. under anaerobic conditions to obtain a hemolytic zone and/or colony and inoculating a liquid media with the hemolytic zone and/or colony and incubating at a temperature of 25-48° C. under anaerobic conditions; and

c) optionally one or more subculturing steps;

d) determining the presence of a Brachyspira sp. Sask30446 polypeptide or polynucleotide in the sample and/or in the hemolytic zone and/or of the colony; and e) isolating the hemolytic zone and/or colony and/or a portion thereof;

wherein the presence of a Brachyspira sp. Sask30446 polypeptide or polynucleotide in the sample and/or the hemolytic zone and/or colony and the presence of hemolytic (e.g. on horse blood), tiny, clear, wet/glistening, “fried egg” colonies or zones of hemolysis on the agar media indicates the extracted hemolytic zone and/or colony comprises Brachyspira sp. Sask30446.

The phrase “Brachyspira sp. Sask30446 polynucleotide” as used herein refers to a polynucleotide having for example at least 92.5% sequence identity to any one of SEQ ID NOs: 7 to 9; a cpn-60 polynucleotide having for example at least 97.5% sequence identity to SEQ ID NO:26; an est polynucleotide having for example at least 93.5% sequence identity with SEQ ID NO:27; a glpk polynucleotide having for example at least 95.5% sequence identity with SEQ ID NO: 29; a pgm polynucleotide having for example at least 93.5% sequence identity with SEQ ID NO: 31 a thi polynucleotide having for example at least 92.5% sequence identity with SEQ ID NO:33; a 16S rRNA polynucleotide having for example, at least 99.5% sequence identity with SEQ ID NO:37 and/or any polynucleotide sequence present in a Brachyspira species comprising any one of SEQ ID NOs:7-9, 25, 27, 29, 31, 33, 35 and 37; and/or which is found in a Brachyspira species associated with haemorrhagic colitis in pigs, wherein the Brachyspira species is not B. pilosicoli and/or B. hyodysenteriae and/or B. murdochii and includes native-sequence polynucleotides, and naturally occurring variants including a portion of a polynucleotide, an isoform, precursor, complex, or modified form and derivatives of the polynucleotide. The polynucleotide can have at least 92.5%, 93.5%, 94.5%, 95.5%, 96.5%, 97.5%, 98.5%, 99% or at least 99.5% or more sequence identity with a sequence of SEQ ID NOs: 7 to 9, 25, 27, 29, 31, 33, 35 and 37. For example, Brachyspira sp. Sask30446 NADPH oxidase polynucleotides have been identified which share about 99% sequence identity. Also included are polynucleotides that encode a Brachyspira sp. Sask30446 polypeptide.

Brachyspira sp. Sask30446 polynucleotides further include sequences that differ from a native sequence due to degeneracy in the genetic code. As one example, DNA sequence polymorphisms within the nucleotide sequence of a Brachyspira sp. Sask30446 NADPH oxidase polynucleotide may result in silent mutations that do not affect the amino acid sequence. Variations in one or more nucleotides may exist among subjects within a population due to natural allelic variation. DNA sequence polymorphisms may also occur which lead to conservative changes in the amino acid sequence of a polypeptide. Fragments are also included.

The term “Brachyspira sp. Sask30446 polypeptide” as used herein refers to a polypeptide comprising at least 92.5%, at least 93.5%, at least 94.5%, at least 96.5%, at least 96.5%, at least 97.5%, at least 98.5%, at least 99% or at least 99.5% sequence identity to SEQ ID NOs: 11, 12, 26, 28, 30, 32, 24, 36, 42, 43, 44 or 45, a polypeptide encoded by any one of SEQ ID NOs: 7 to 9, 25, 27, 29, 31, 33, 35 or 37, any polypeptide expressed in a Brachyspira species comprising any one of SEQ ID NOs: 11, 12, 26, 28, 30, 32, 24, 36 42, 43, 44 or 45, and/or in a Brachyspira species associated with haemorrhagic colitis in pigs, wherein the Brachyspira species is not B. pilosicoli and/or B. hyodysenteriae and/or B. murdochii and one or more of the phenotypic characteristics described in Tables 4 and/or 5 and in particular includes the native-sequence polypeptide, isoforms, all homologs, fragments, precursors, complexes, and modified forms and derivatives thereof. For example, a Brachyspira sp. Sask30446 NADPH oxidase polypeptide sequence comprises 270 amino acids (encoded by 810 nucleotides) and is 92.3% identical (97% similar) to both EF517544 Brachyspira innocens and DQ487124 Brachyspira suanatina.

In an embodiment, the inoculum is an agar section comprising Brachyspira sp. Sask30446. Accordingly, in an embodiment, the method further comprises culturing the Brachyspira sp. Sask30446 on solid agar to provide an inoculum (e.g. a hemolytic zone) for inoculating a liquid media.

As mentioned, the presence of a Brachyspira sp. Sask30446 polypeptide or polynucleotide in the sample and/or the hemolytic zone and/or colony and/or incubated culture and the presence of hemolytic (e.g. on horse blood), tiny, clear, wet/glistening, “fried egg” colonies or zones of hemolysis on the solid agar media and/or one or more of the phenotypic characteristics described in Tables 4 and/or 5 indicates the extracted hemolytic zone and/or colony comprises Brachyspira sp. Sask30446.

A person skilled in the art would recognize that the identity of the organism can be confirmed at any step, and need be confirmed only at one step, for example at the stage of obtaining a hemolytic zone on a solid agar plate and/or after isolating the inoculating and incubated liquid media to confirm the identity of the Brachyspira sp., for example Brachyspira sp. Sask30446. For example, the sample and/or inoculum can be confirmed to comprise Brachyspira sp. Sask30446. The sample and/or inoculum can be used to inoculate a solid agar media and/or liquid media culture.

The bacterial isolates are preferably purified and their identity determined and/or confirmed using methods disclosed herein and/or in PCT/CA2011/000828 titled DIAGNOSTIC METHOD FOR COLITIS filed Jul. 18, 2011.

In an embodiment, the solid media is BJ, CVS or blood agar.

In an embodiment, the liquid media comprises BHI and/or HI broth, glucose and whole blood. In another embodiment, the liquid media further comprises serum.

In an embodiment, the method further provides Brachyspira sp. Sask30446 phenotypic information, which can be used and/or can aid in determining whether a subject suspected of being infected with Brachyspira sp. Sask30446 is infected, the presence of a zone of hemolysis on a BJ or CVS agar media, such as strong 8-hemolysis and/or one or more characteristics in Table 4 and/or 5, is indicative the subject is infected with Brachyspira sp. Sask30446. Phenotypic information can be used in conjunction with the methods in PCT/CA2011/000828 titled DIAGNOSTIC METHOD FOR COLITIS filed Jul. 18, 2011 and/or described below.

The term “subject” as used herein refers to any member of the animal kingdom, preferably a mammal, more preferably a member of the swine family, including pigs, hogs and boars or a member of the avian family such as geese.

As used herein and in the appended claims, the singular forms “a,” “an,” and “the” include plural reference unless the context clearly dictates otherwise. As well, the terms “a” (or “an”), “one or more,” “at least one,” “comprising,” “including,” “characterized by” and “having” can be used interchangeably herein.

In an embodiment, the method of screening for or detecting the presence of Brachyspira sp. Sask30446 organism in a sample from a subject using for example a culturing method described herein comprises:

a) obtaining a sample from the subject;

b) inoculating a solid media and/or liquid media with the sample and/or an inoculum derived from the sample and incubating at a temperature of 25-44° C. under anaerobic conditions, wherein the solid media is BJ or CVS solid agar media; for sufficient time to obtain one or more hemolytic zone.

c) inoculating a liquid media with the hemolytic zone;

d) detecting a Brachyspira sp. Sask30446 polynucleotide or polypeptide in any cell obtained in step b) or c);

wherein the presence of a Brachyspira sp. Sask30446 polynucleotide or polypeptide is indicative of the presence of Brachyspira sp. Sask30446 in the subject.

Methods of detecting Brachyspira sp. Sask30446 polynucleotide or polypeptide are disclosed in PCT/CA2011/000828 titled DIAGNOSTIC METHOD FOR COLITIS filed Jul. 18, 2011 and/or described below.

Another embodiment provides an isolated Brachyspira sp. Sask30446 organism. In an embodiment, the isolated Brachyspira sp. Sask30446 is obtained by a method described herein (e.g. using a solid media or liquid media described herein). The solid agar culture methods and/or obtaining of hemolytic zones and/or very small colonies. The broth culture methods for example permit scaled propagation and isolation of Brachyspira sp. Sask30446, particularly where combined with a prior step of purifying the Brachyspira sp. Sask30446 organism using solid media, such as BJ or CVS on which after about 24-48 hours of incubation of hemolytic zones can be visible. In an embodiment, the isolated Brachyspira sp. Sask30446 comprises one or more sequences of SEQ ID NOs: 7, 8 9, 11, 12, 25-34 and 37; or at least 92.3% sequence identity to any one of SEQ ID NOs: 7 to 9, 11 and 12; at least 97.5% sequence identity to SEQ ID NO: 25; at least 98.5% sequence identity to SEQ ID NO:26; at least 93.5% sequence identity to SEQ ID NO:27 or 31; at least 95.5% sequence identity to SEQ ID NO:28 or 29 or 34; at least 99.5% sequence identity to SEQ ID NO: 30 or 32 or 37; at least 92.5% sequence identity to SEQ ID NO:33; and/or any combination thereof. In an embodiment, the Brachyspira sp. Sask30446 comprises one or more polynucleotides or polypeptides with at least 97%, 98% or 99% sequence identity to one or more of SEQ ID NOS: 7-9, 11, 12, 25-29, 31, 33-34 and/or at least 99.5% sequence identity to one or more of SEQ ID NOS:30, 32 or 37.

In another embodiment, the isolated Brachyspira sp. Sask30446 organism comprises one or more sequences of SEQ ID NO: 42, 43, 44, and/or 45, for example comprising a polypeptide comprising one or more sequences of SEQ ID NO: 42, 43, 44, and/or 45 and/or a nucleic acid molecule encoding one or more sequences of SEQ ID NO: 42, 43, 44, and/or 45; or one or more sequences having at least 92.5% sequence identity with SEQ ID NO: 42, 95.5% sequence identity with SEQ ID NO:43; at least 96.5% sequence identity with SEQ ID NO:44 and/or 94.5% sequence identity with SEQ ID NO: 45 and/or any combination thereof.

The above sequences are Brachyspira sp. Sask30446 polynucleotide or polypeptide sequences including NADPH oxidase (nox1) (polynucleotide: SEQ ID NOs:7-9; polypeptide: SEQ ID NOs:11-12), chaperonin 60 (cpn60) (polynucleotide: SEQ ID NO:25; polypeptide: SEQ ID NO:26), esterase (est) (polynucleotide: SEQ ID NO:27; polypeptide: SEQ ID NO:28), glucose kinase (glpk) (polynucleotide: SEQ ID NO:29; polypeptide: SEQ ID NO:30), phosphoglucomutase (pgm) (polynucleotide: SEQ ID NO:31; polypeptide: SEQ ID NO:32), acetyl-CoA acetyltransferase (thi) (polynucleotide: SEQ ID NO:33; polypeptide: SEQ ID NO:34) and small subunit ribosomal RNA (16S rRNA) (polynucleotide: SEQ ID NO:37). Also provided is HLY1 (SEQ ID NO: 42), HLY2 (SEQ ID NO:43), HLY3 (or hlyB) (SEQ ID NO: 44) and HLY4 (also hlyC)(SEQ ID NO:45).

In an embodiment, the isolated Brachyspira sp. comprises a DNA genome encoding one or more polypeptides selected from SEQ ID NO: 11, 12, 26, 28 30, 32, 34, 42, 43, 44 and/or 45; SEQ ID NOs 11, 12, 26, 28, 30, 32, 34 42, 43, 44 and/or 45 and/or one or more polypeptides selected from polypeptides with sequences that have at least 92.3% sequence identity with SEQ ID NO: 11, 12 or 42, at least 98.5% sequence identity with SEQ ID NO:26; at least 95.5% sequence identity with SEQ ID NO:28, 34 or 43; at least 99.5% sequence identity with SEQ ID NO: 30 or 32, at least 96.5% sequence identity with SEQ ID NO:44 and/or 94.5% sequence identity with SEQ ID NO: 45 and/or any combination thereof. The term “sequence identity” alternatively referred to as “identity” as used herein refers to the percentage of sequence identity between two polypeptide sequences and/or two polynucleotide sequences, for which methods of determining are known in the art. For example, in order to determine the percentage of identity between two polypeptide sequences, the amino acid sequences of such two sequences are aligned, preferably using the Clustal W algorithm (Thompson, J D, Higgins D G, Gibson T J, 1994, Nucleic Acids Res. 22 (22): 4673-4680), together with BLOSUM 62 scoring matrix (Henikoff S. and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990: 215: 403).

“Percent sequence identity” of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An isolated organism can be assayed to determine if it is Brachyspira sp. Sask30446 by for example amplifying a polynucleotide of the isolated organism to be assayed as described herein and as in PCT/CA2011/000828 titled DIAGNOSTIC METHOD FOR COLITIS filed Jul. 18, 2011 and comparing the sequence to one or more of the Brachyspira sp. Sask30446 sequences provided herein. The presence of one or more sequences of SEQ ID NOs: 7, 8 9, 11, 12, 25-34 and 37; or a sequence with at least 92.3% sequence identity to any one of SEQ ID NOs: 7 to 9, 11 and 12; at least 97.5% sequence identity to SEQ ID NO: 25; at least 98.5% sequence identity to SEQ ID NO:26; at least 93.5% sequence identity to SEQ ID NO:27 or 31; at least 95.5% sequence identity to SEQ ID NO:28 or 29 or 34; at least 99.5% sequence identity to SEQ ID NO: 30 or 32 or 37; at least 92.5% sequence identity to SEQ ID NO:33; and/or any combination thereof, is indicative of the organism being Brachyspira sp. Sask30446. Further confirmation can be obtained by analyzing the growth properties of the isolated organism. An isolate with the requisite sequence identity and growth properties as described herein, is indicative that the isolated organism is Brachyspira sp. Sask30446. In an embodiment, the sequence is at least about 97%, 98%, or 99% identical to one more of SEQ ID NOs: 7, 8 9, 11, 12, 25-29, 31, 33 and 34 and/or at least 99.5% identical to one or more of SEQ ID NOs: 30, 32 and 37.

In an embodiment the isolated Brachyspira sp. Sask30446 is a gram-negative, anaerobic, spirochete bacterium. In an embodiment, the isolated Brachyspira sp. B comprises the characteristics described in Table 4 and/or 5.

In an embodiment, the isolated Brachyspira sp. is characterized by the properties of the bacteria strain deposited with the International Depository of Canada (1015 Arlington St., Suite H3390 Winnipeg, MB R3E 3R2) on Nov. 16, 2011 under Accession number 16111-01. In yet a further embodiment, the isolated Brachyspira sp. Sask30446 comprises genomic sequence with at least 95%, 96%, 97%, 98%, 99%, or at least 99.5% sequence identity to the bacteria strain deposited with the International Depository of Canada (IDAC) (1015 Arlington St., Suite H3390 Winnipeg, MB R3E 3R2) on Nov. 16, 2011 under Accession number 16111-01.

In an embodiment, the isolated Brachyspira sp. is the strain deposited with IDAC Accession number 161111-01 and/or is a progeny and/or immunologically active derivative of the Brachyspira sp. Sask30446 strain deposited under Accession number 16111-01.

A further aspect includes a microbiological culture comprising Brachyspira sp. Sask30446, grown according to a method described herein using for example a solid media or liquid media described herein.

A further embodiment includes an isolated Brachyspira sp. Sask30446 obtainable by a method or process described herein. For example, a sample comprising or suspected of comprising Brachyspira sp. Sask30446 is obtained from a subject, the sample is cultured according to a method described herein using for example a media disclosed herein and the sample and/or cultured organism is tested for one or more Brachyspira sp. Sask30446 polypeptides or polynucleotides (e.g. disclosed below). If positive, a colony and/or hemolytic zone is extracted from the agar and/or solid media.

A further aspect concerns a method of preparing the isolated Brachyspira sp. Sask30446 organism. In an embodiment, the Brachyspira sp. Sask30446 is cultured according to a method described herein and the Brachyspira sp. Sask30446 is extracted from the solid media and/or a portion thereof, for example from the agar media, for example by removing a colony or cutting a portion of the solid agar comprising a hemolytic zone isolated from the solid media (e.g. extracted from the solid media plate, contaminants etc). The isolated Brachyspira sp. Sask30446 can be used to inoculate a liquid media, inoculated as described. The organism can be isolated from the liquid culture.

In an embodiment, the method of isolating a Brachyspira sp. Sask30446 organism from a sample comprises culturing a sample comprising Brachyspira sp. Sask30446 according to a method described herein; and extracting the Brachyspira sp. Sask30446 organism from the solid and/or liquid media.

In an embodiment, the Brachyspira sp. Sask30446 is extracting by cutting a portion of the solid media comprising a hemolytic zone.

In another embodiment, the Brachyspira sp., such as Brachyspira sp. Sask30446, is isolated by separating the organism from the liquid media, for example by centrifuging the incubated inoculated liquid media according to a method known in the art.

The isolated colony or agar portion can be used to further propagate the organism (e.g. subculturing onto a fresh growth media plate or liquid media culture).

In an embodiment, the isolated colony or agar portion is used to inoculate a starter liquid culture and the starter culture is used to inoculate a liquid media.

In an embodiment, Brachyspira sp. Sask30446 is frozen down, for example by freezing an agar plate, slant, etc or an agar portion (e.g. slice) at about −80° C., for example, by placing the portion in a sterile vial such as a cryo vial or freezing the plate comprising a hemolytic zone and/or colony obtained using a culturing method described herein. In an embodiment, the sample used in the method of culturing Brachyspira sp. Sask30446 comprises thawing an agar plate, plug or slice comprising a Brachyspira sp. Sask30446 hemolytic zone and incubating the plate and/or inoculating a solid media with the thawed agar plug.

In another embodiment, Brachyspira sp. Sask30446 is frozen down by resuspending the isolated Brachyspira sp. Sask30446 in a suitable freezing media, comprising for example DMSO or other cryoprotectant.

In an embodiment, a culture of Brachyspira sp. Sask30446 is frozen at for example −80° C., for example in liquid media such as JBS media.

A further aspect includes a liquid media composition for propagating Brachyspira sp. Sask30446.

In an embodiment, the liquid media composition comprises BHI and/or HI, a blood product such as ovine blood, for example at a concentration disclosed herein (e.g. 1-20% v/v of the composition) and glucose for example at a concentration disclosed herein e.g. 0.5% to 10% v/v of the composition. The liquid media composition can for example be powdered and later reconstituted. In yet another embodiment, the liquid media composition comprises calf blood product such as fetal calf blood product as also disclosed herein (e.g. 1-20%) and can for example be deactivated using for example methods known in the art (e.g. heat inactivation).

In an embodiment, the blood product is whole blood. In another embodiment, the blood product is serum. In another embodiment, the media comprises a mixture of whole blood and serum.

Another aspect, includes a process of producing a Brachyspira sp. Sask30446 composition such as an immunogenic composition comprising:

a) inoculating a liquid media with an inoculum according to a method described herein, and

b) incubating the inoculated liquid media at a temperature of 25-44° C. under anaerobic conditions;

c) optionally one or more subculturing steps;

d) optionally confirming the identity/presence of a Brachyspira sp. Sask30446 polypeptide or polynucleotide in the sample;

e) isolating the Brachyspira sp. Sask30446; and

f) combining the composition of step e) with a carrier or diluent.

In an embodiment, the inoculum is an agar section comprising Brachyspira sp. Sask30446. Accordingly, in an embodiment, the method further comprises a prior step of culturing the Brachyspira sp. Sask30446 on solid agar to provide an inoculum (e.g. a hemolytic zone) for inoculating the liquid media.

In an embodiment, the isolated Brachyspira sp. Sask30446 is inactivated. In an embodiment, Brachyspira sp. Sask30446 is inactivated prior to step f) (e.g. prior to combining with a carrier or diluent, or after step f). Various physical and chemical methods of bacterial inactivation are known in the art. Examples are UV-radiation, X-ray radiation, gamma-radiation and heating. Examples of inactivating chemicals are beta-propiolactone, glutaraldehyde, beta-ethyleneimine, hypochlorite and formaldehyde. Other methods of inactivating the bacteria are known to the skilled person.

The term “inactivated” as used herein means that the bacterium is killed and incapable of replication and causing clinical disease.

In another embodiment, the Brachyspira sp. Sask30446 is live attenuated obtainable by a method known in the art.

The term “attenuated” as used herein means that the bacterium is live and may be capable of replicating without typically causing clinical disease.

In an embodiment, the inactivated or attenuated Brachyspira sp. Sask30446 has the characteristics of the strain deposited with the International Depository of Canada (1015 Arlington St., Suite H3390 Winnipeg, MB R3E 3R2) on Nov. 16, 2011 under Accession number 16111-01 and/or comprises a progeny and/or immunologically active derivative of the Brachyspira sp. Sask30446 strain deposited under Accession number 16111-01.

In another embodiment, the disclosure provides an immunogenic fragment or fraction of Brachyspira sp. Sask30446 organism obtainable by a method known in the art. For example, a soluble fraction can be obtained by detergent solubilization of Brachyspira sp. Sask30446. The fractions and/or fragments can be for example purified outer membrane antigens from Brachyspira sp. Sask30446, such as outer membrane proteins. In an embodiment, the fraction is an outer membrane fraction. In an embodiment, the fraction is a soluble fraction.

A “fragment” of Brachyspira sp. Sask30446, as used herein is any immunogenic component or part of Brachyspira sp. Sask30446 such as a polypeptide comprising at least one antigenic determinant.

A further aspect includes a composition comprising an isolated Brachyspira sp. Sask30446 organism and/or a fragment or fraction thereof in admixture with a suitable diluent or carrier, such as a veterinarily suitable diluent or carrier or a pharmaceutically suitable diluent or carrier. In an embodiment, the composition comprises an isolated Brachyspira sp. Sask30446 organism. The isolated Brachyspira sp. Sask30446, can be live, attenuated and/or killed and can be isolated and/or cultured using a method described herein. In an embodiment, the composition further includes an isolated polypeptide of Brachyspira sp. Sask30446, including for example one or more polypeptides of SEQ ID NO:11, 12, 26, 28, 30, 32, 34, 42, 43, 44 and 45; a polypeptide having at least 92.3% sequence identity to any one of SEQ ID NOs: 11, 12 and 42; at least 98.5% sequence identity to SEQ ID NO: 26; at least 95.5% sequence identity to SEQ ID NO: 28, 34 or 43; at least 99.5% sequence identity to SEQ ID NO: 30; at least 99.5% sequence identity to SEQ ID NO: 32; at least 96.5% sequence identity to SEQ ID NO:44 and/or 94.5% sequence identity to SEQ ID NO: 45 and/or any combination thereof; or a combination of two or more thereof. In an embodiment, the composition is an immunogenic composition comprising one or more of Brachyspira sp. Sask30446 organism an isolated polypeptide of Brachyspira sp. Sask30446, including for example one or more polypeptides of SEQ ID NO:11, 12, 26, 28, 30, 32, 34, 42, 43, 44 and 45; a polypeptide having at least 92.3% sequence identity to any one of SEQ ID NOs: 11, 12 and 42; a polypeptide having at least 98.5% sequence identity to SEQ ID NO: 26; a polypeptide having at least 95.5% sequence identity to SEQ ID NO: 28, 34 or 43; a polypeptide having at least 99.5% sequence identity to SEQ ID NO: 30; a polypeptide having at least 99.5% sequence identity to SEQ ID NO: 32; and/or a polypeptide having at least 96.5% identity to SEQ ID NO:44 and/or at least 94.5% sequence identity to SEQ ID NO: 45 and/or any combination thereof; or a combination of two or more thereof, optionally formulated with an adjuvant.

In an embodiment, the sequence identity (e.g. of the polypeptide, polynucleotide and/or organism genome) is at least at least 98%, 98.5%, 99% or at least 99.5% sequence identity to SEQ ID NO: 25; at least 99% or at least 99.5% sequence identity to SEQ ID NO:26; at least 94%, 94.5%, 95%, 95.5%, 96%, 96.5%, 97%, 97.5%, 98%, 98.5%, 99% or at least 99.5% sequence identity to SEQ ID NO:27, 31 or 45; at least 95.5%, 96%, 96.5%, 97%, 97.5%, 98%, 98.5%, 99% or at least 99.5% sequence identity to SEQ ID NO:28, 29, 34, 43 or 45; at least 99.5% or at least 99.8% sequence identity to SEQ ID NO: 30 or 32 or 37; at least 92.5%, 93%, 93.5%, 94%, 94.5%, 95%, 95.5%, 96%, 96.5%, 97%, 97.5%, 98%, 98.5%, 99% or at least 99.5% sequence identity to SEQ ID NO:33 or 42; and/or any combination thereof.

In an embodiment, the composition comprises the Brachyspira sp. Sask30446 strain deposited with the International Depository of Canada (1015 Arlington St., Suite H3390 Winnipeg, MB R3E 3R2) on Nov. 16, 2011 under Accession number 16111-01 or a strain with similar characteristics (e.g. sequence etc.). The strain can for example be live e.g. live culture, attenuated and/or killed. In an embodiment, the composition comprises a progeny and/or immunologically active derivative of the Brachyspira sp. Sask30446 strain deposited under Accession number 16111-01.

In an embodiment, the composition is a liquid composition, for example for direct inoculation, admixture with drinking water and/or for adding to feed. In an embodiment the composition is a solid composition, for example for admixing with feed and/or in an admixture with feed. In an embodiment, the composition is a liquid Brachyspira sp. Sask30446 immunogenic composition comprising a Brachyspira sp. Sask30446 described herein and a diluent or carrier. In an embodiment, the composition is a liquid Brachyspira sp. Sask30446 immunogenic composition comprising a Brachyspira sp. Sask30446 described herein and carrier.

As described herein, compositions comprising Brachyspira sp. Sask30446 are immunogenic giving rise for example to the production of antibodies and/or cell mediated immunity upon inoculation. Accordingly, an embodiment comprises an immunogenic composition.

In an embodiment, the composition is an immunogenic composition comprising live and/or live attenuated and/or inactivated Brachyspira sp. Sask30446. In an embodiment, the immunogenic composition comprises an immunogenic fraction or immunogenic fragment of Brachyspira sp. Sask30446.

Suitable diluents include for example a buffered solution, saline aqueous solvent, non-aqueous solvent and/or the like. The composition can also comprise a preservative.

In yet a further embodiment, the composition is a pharmaceutical composition wherein the suitable diluent or carrier is a pharmaceutically suitable diluent or carrier.

Suitable pharmaceutically acceptable carriers known in the art include, but are not limited to, gold particles, sterile water, saline, glucose, dextrose or buffered solutions.

Carriers may include auxiliary agents including, but not limited to, diluents, stabilizers (i.e., sugars and amino acids), preservatives, wetting agents, emulsifying agents, pH buffering agents, viscosity enhancing additives, colors and the like. In general, a diluent or carrier is selected on the basis of the mode and route of administration, and standard pharmaceutical and/or veterinary practice.

The compositions described herein can be prepared by known methods for the preparation of pharmaceutically acceptable compositions that can be administered to subjects, such that an effective quantity of the active substance is combined in a mixture with acceptable diluents or carriers. Suitable diluents and carriers are described, for example, in Remington's Pharmaceutical Sciences (Remington's Pharmaceutical Sciences, 20^{th} ed., Mack Publishing Company, Easton, Pa., USA, 2000). On this basis, the compositions include, albeit not exclusively, solutions of the substances in association with one or more pharmaceutically acceptable diluents or carriers, and contained in buffered solutions with a suitable pH and iso-osmotic with the physiological fluids.

The composition can be for example desiccated or lyophilized.

In an embodiment, the composition and/or isolated Brachyspira sp. Sask30446 is thermally inactivated, dessicated. lyophilized, spray-dried, or dried.

In an embodiment, the composition and/or isolated Brachyspira sp. Sask30446 are provided in a kit for example with a diluent for rehydrating the composition e.g. vaccine composition. Accordingly another aspect comprises a kit comprising an isolated Brachyspira sp. Sask30466 and a resuspension diluent. The kit can comprise one or more components for example a container such as a sterile vial for containing the composition and instructions for use.

An “immunogenic composition” as used herein means a composition that induces an immune response, for example induces an antibody response.

In an embodiment, the immunogenic composition is for use in or as a vaccine, e.g. a vaccine composition.

In an embodiment, the composition is a vaccine composition.

The term “vaccine” or “vaccine composition” as used herein is a vaccine for veterinary use comprising antigenic substances and is administered for the purpose of inducing a specific and active or passive immunity against a disease provoked by Brachyspira sp. Sask30446.

As described below, pigs inoculated with high doses of Brachyspira sp. Sask30446 often became sick. Attenuated strains for example would be expected to induce protective immunity.

Immunogenicity can be significantly improved if the immunizing agent (i.e. the isolated Brachyspira sp. Sask30446 organism and/or fraction or fragment thereof) and/or composition is, regardless of administration format, co-immunized with an adjuvant. Commonly, adjuvants are used as a 0.05 to 1.0 percent solution in phosphate buffered saline. Adjuvants enhance the immunogenicity of an immunogen but are not necessarily immunogenic in and of themselves. Adjuvants may act by retaining the immunogen locally near the site of administration to produce a depot effect facilitating a slow, sustained release of immunogen to cells of the immune system. Adjuvants can also attract cells of the immune system to an immunogen depot and stimulate such cells to elicit immune response. As such, embodiments encompass compositions and pharmaceutical compositions further comprising adjuvants.

Adjuvants have been used for many years to improve the host immune responses to, for example, vaccines. Intrinsic adjuvants (such as lipopolysaccharides) normally are the components of killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Aluminum hydroxide and aluminum phosphate (collectively commonly referred to as alum) are for example routinely used as adjuvants in human and veterinary vaccines.

A wide range of extrinsic adjuvants can provoke potent immune responses to immunogens. These include saponins complexed to membrane protein antigens (immune stimulating complexes), pluronic polymers with mineral oil, killed mycobacteria and mineral oil, Freund's complete adjuvant, Incomplete Freund's adjuvant, bacterial products such as muramyl dipeptide (MDP) and lipopolysaccharide (LPS), as well as lipid A, and liposomes. Other suitable adjuvants include for example CpG, peanut oil and oilwater mixtures for example sold as Amphigen™ (Pfizer) and Immunostim™ (Bioniche).

In an embodiment, the adjuvant is selected from aluminum compounds (such as aluminum hydroxide, aluminum phosphate, and aluminum hydroxy phosphate). The antigen can be precipitated with, or adsorbed onto, the aluminum compound according to standard protocols. Other adjuvants such as RIBI (ImmunoChem, Hamilton, Mont.) can also be used. In another embodiment, the adjuvant is selected from bacterial toxins (e.g., the cholera toxin (CT), the E. coli heat-labile toxin (LT), the Clostridium difficile toxin A and the pertussis toxin (PT), or combinations, subunits, toxoids, or mutants thereof). Other adjuvants (such as a bacterial monophosphoryl lipid A (MPLA) of various sources (e.g., E. coli, Salmonella minnesota, Salmonella typhimurium, or Shigella flexneri, saponins, or polylactide glycolide (PLGA) microspheres) can also be used.

A further aspect is the use of the isolated Brachyspira sp. Sask30446 or a composition of the disclosure comprising isolated Brachyspira sp. Sask30446, or an isolated fragment or fraction thereof such as a polypeptide of Brachyspira sp. Sask30446, optionally including for example one or more polypeptides of SEQ ID NO:11, 12, 26, 28, 30, 32 and 34; a polypeptide having at least 92.3% sequence identity to any one of SEQ ID NOs: 11 and 12; a polypeptide having at least 98.5% sequence identity to SEQ ID NO: 26; a polypeptide having at least 95.5% sequence identity to SEQ ID NO: 28; a polypeptide having at least 99.5% sequence identity to SEQ ID NO: 30; a polypeptide having at least 99.5% sequence identity to SEQ ID NO: 32; and/or a polypeptide having at least 95.5% sequence identity to SEQ ID NO: 34; or a combination of two or more thereof, to induce an immune response in a subject.

Also included in another aspect is a method for inducing an immune response in a subject against the isolated Brachyspira sp. Sask30446 organism and/or fraction or fragment thereof such as an isolated polypeptide of any one of SEQ ID NO:11, 12, 26, 28, 30, 32 and 34; a polypeptide having at least 92.3% sequence identity to any one of SEQ ID NOs: 11 and 12; a polypeptide having at least 98.5% sequence identity to SEQ ID NO: 26; a polypeptide having at least 95.5% sequence identity to SEQ ID NO: 28; a polypeptide having at least 99.5% sequence identity to SEQ ID NO: 30; a polypeptide having at least 99.5% sequence identity to SEQ ID NO: 32; and/or a polypeptide having at least 95.5% sequence identity to SEQ ID NO: 34; or a combination of two or more thereof, comprising administering to the subject or a cell from the subject an effective amount of the isolated Brachyspira sp. Sask30446 organism or the isolated polypeptide.

A person skilled in the art would readily know how to prepare polynucleotides and polypeptides of isolated Brachyspira sp. Sask30446 given the teachings of the present disclosure (e.g. sequence data). For example, Brachyspira sp. Sask30446 a polynucleotide can be prepared by cloning the nucleotide sequence and a Brachyspira sp. Sask30446 polypeptide can be prepared by for example expressing the cloned nucleotide sequence in a suitable cell.

Administering” or “administration” includes any means for introducing the immunogenic composition into a subject. Examples include but are not limited to oral, buccal, sublingual, pulmonary, transdermal, and transmucosal delivery, as well as subcutaneous, intraperitoneal, intravenous, and intramuscular injection. Oral delivery can include for example administering the composition by oral bolus, or through a water system and/or a food system, which is for example provided to subjects (e.g. pigs) for example in group pens. In addition, the composition can be administered all at once, as for example, by a bolus injection; multiple times, such as by a series of tablets; or delivered substantially uniformly over a period of time, as for example, using transdermal delivery. Further, the dose of the compound can be varied over time. A compound can be administered using an immediate release formulation, a controlled release formulation, or combinations thereof. The term “controlled release” includes sustained release, delayed release, and combinations thereof.

The term “a cell” includes a single cell as well as a plurality or population of cells. Administering an agent to a cell includes both in vitro and in vivo administrations.

The term “eliciting an immune response” or “inducing an immune response” as used herein means initiating, triggering, causing, enhancing, improving or augmenting any response of the immune system, for example, of either a humoral or cell-mediate nature. The initiation or enhancement of an immune response can be assessed using assays known to those skilled in the art including, but not limited to, antibody assays (for example ELISA assays), antigen specific cytotoxicity assays and the production of cytokines (for example ELISPOT assays).

The term “isolated protein” refers to a protein substantially free of cellular material or culture media when produced by recombinant DNA techniques, or chemical precursors or other chemicals when chemically synthesized.

A subject may be immunized with a composition e.g. pharmaceutical composition, comprising an isolated Brachyspira sp. Sask30446; and optionally further including an isolated polypeptide thereof, the isolating polypeptide selected from SEQ ID NO: 11, 12, 26, 28, 30, 32, 34, 42, 43, 44 and 45; or having at least 92.3% sequence identity to any one of SEQ ID NOs: 11, 12 and 42; at least 98.5% sequence identity to SEQ ID NO: 26; at least 95.5% sequence identity to SEQ ID NO: 28 or 43; at least 99.5% sequence identity to SEQ ID NO: 30; at least 99.5% sequence identity to SEQ ID NO: 32; at least 95.5% sequence identity to SEQ ID NO: 34; at least 96.5% identity to SEQ ID NO:44 and/or 94.5% identity to SEQ ID NO: 45; or a combination of two or more thereof, by any conventional route as is known to one skilled in the art. This may include, for example, immunization via a mucosal (e.g., ocular, intranasal, oral, gastric, pulmonary, intestinal, rectal, vaginal, or urinary tract) surface or via the parenteral (e.g., subcutaneous, intradermal, intramuscular, intravenous, or intraperitoneal) route. Preferred routes depend upon the choice of the immunogen as will be apparent to one skilled in the art. The administration can be achieved in a single dose or repeated at intervals. The appropriate dosage depends on various parameters understood by skilled artisans such as the immunogen itself (i.e. peptide vs. Brachyspira sp. Sask30446 organism), the route of administration and the condition of the animal to be vaccinated (weight, age and the like).

A person skilled in the art will appreciate that the compositions can be formulated for administration to subjects in a biologically compatible form suitable for administration in vivo. The substances may be administered to living organisms including humans and animals. Administration of a therapeutically active amount of the pharmaceutical compositions is defined as an amount effective, at dosages and for periods of time necessary, to achieve the desired result.

The composition e.g. pharmaceutical composition may be administered systemically. Depending on the route of administration, the pharmaceutical composition may be coated in a material to protect the composition from the action of enzymes, acids and other natural conditions that may inactivate the compound.

The compositions can be administered to swine, including healthy swine, for example as early as at about 3 weeks of age

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Isolating Brachyspira sp. Sask30446

In an embodiment, Brachyspira sp. Sask30446 is isolated from a sample obtained from an infected subject. For example, an animal with severe mucohaemorrhagic typhlocolitis unrelated to B. hyodysenteriae and B. pilosicoli (Bh and Bp) (e.g. in tissues and/or feces) can be a source of Brachyspira sp. Sask30446. An infected tissue sample or fecal swab is cultured according to a method described herein e.g. using a solid media such as BAM-SR, CVS of BJ, the sample and/or growth on the agar media is confirmed to comprise the Brachyspira sp. Sask30446 polynucleotide or polypeptide and the hemolytic zone and/or colony comprising Brachyspira sp. Sask30446 is extracted from the agar media, and optionally frozen or used for example to inoculate a subsequent agar media.

The sample can be obtained from a subject suspected to be infected with Brachyspira sp. Sask30446, based on for example phenotypic presentation and/or genotypic testing. For example detailed necropsy examinations on two affected and two non-affected pigs found that the affected pigs demonstrated bloody diarrhea, whereas the age-matched, non-affected pigs demonstrated normal feces. The pathologic lesions in the 2 clinical cases were typical of swine dysentery: necrotizing haemorrhagic non-suppurative colitis+/− typhlitis. There were no remarkable lesions in the non-affected pigs. The Brachyspira sp. was identified on direct examination of colon of the affected but not of the non-affected pigs. The Brachyspira species Sask30446 was detected by PCR in the colonic tissue of only affected pigs. Testing for B. hyodysenteriae and B. pilosicoli was completed and shown to be negative for all pigs for all tissues. Other known intestinal swine pathogens including Lawsonia intracellularis, rotavirus, transmissible gastroenteritis and porcine circovirus were ruled out as was Porcine Reproductive and Respiratory Syndrome (PRRS) virus. Accordingly a sample from an animal with these characteristics can be cultured, confirmed to be Brachyspira sp. Sask30446 and extracted from a solid agar as described.

a Method of Detecting Presence of Brachyspira Sp. Sask30446 Polynucleotide

The presence of Brachyspira sp. Sask30446 can be detected in a sample by using SEQ ID NO: 1 and 2 or SEQ ID NO: 3 and 4 to amplify an approximately 241 base pair sequence. As these primers are specific for Brachyspira sp. Sask30446, amplification is indicative that the sample comprises Brachyspira sp. Sask30446. This 241 base pair sequence has been amplified in several different isolates. The isolates share about 99% sequence identity over the 241 base pair sequence. For example, SEQ ID NO: 7 and SEQ ID NO: 8 differ at positions 11 and 12.

Bnox sequence PCR primers (sequence below) can amplify a 241 bp fragment of the Brachyspira sp. Sask30446 sequence.

BnoxF 5′-TAG CYT GCG GTA TYG CWC TTT GG-3′ [JH0222] (SEQ ID NO:1) and BnoxR 5′-CTT CAG ACC AYC CAG TAG MG CC-3′ [JH0223] (SEQ ID NO:2), where W=A or T and Y=C or T.

A second set of primers that can be used is SEQ ID NO:3 and 4: 5′-TCG CTA MT TAT TCC MC MG GA-3′ [JH0224] (SEQ ID NO:3) and 5′-MA CGC ATT TCT ATT CCA GCA-3′ [JH0225] (SEQ ID NO:4)

The primers SEQ ID NO: 5 and 6 which amplify an approximate 184 base pair sequence can alternatively be used to detect the presence of Brachyspira sp. Sask30446.

Primer sequence and examples of Brachyspira sp. Sask30446—184 bp amplification product sequences are provided:

(SEQ ID NO: 5)
JH0249 5′-TCATAGATGCTGGAATAGAAATGC-3′
(SEQ ID NO: 6)
JH0250 5′-GCACCGTTAGGTAAAGTTTCCA-3′
>TM25_H02_2478-cnf_002 to.0.2
SEQ ID NO: 7
TCGCTAAATTTATCCAACAAGGACAGGATATCATTAATGAAATAGCTAAA
CCTGAAGTAAAAAAAGTTATGGTTGTTGGTGCTGGTTATATAGGTGTTGA
GCTTATAGAAGCATTCAAAAACCATGGTAAAGAAGTTATCTTAATGGAAT
CTATGCCTAGAGTTATGGCTAACTATTTTGATAAAGAAATCACTGATGAA
GCTGAAAAAAGAATCATAGATGCTGGAATAGAAATGCGTTT
>TM25_H06_2482-cnf_034 to.0.2
SEQ ID NO: 8
TCGCTAAATTATTCCAACAAGGACAGGATATCATTAATGAAATAGCTAAA
CCTGAAGTAAAAAAAGTTATGGTTGTTGGTGCTGGTTATATAGGTGTTGA
GCTTATAGAAGCATTCAAAAACCATGGTAAAGAAGTTATCTTAATGGAAT
CTATGCCTAGAGTTATGGCTAACTATTTTGATAAAGAAATCACTGATGAA
GCTGAAAAAAGAATCATAGATGCTGGAATAGAAATGCGTTT

### Materials and Methods for PCR Detection

Tissue samples were weighed prior to DNA extraction to facilitate quantification. Genomic DNA was extracted from tissue samples using the Blood & Tissue Extraction Kit (Qiagen) according to manufacturer's instructions. Genomic DNA was extracted from feces or intestinal contents using the QIAamp DNA Stool Mini Kit (Qiagen). All assay reaction mixtures consisted of 1× iQ SYBR green supermix (Bio-Rad), 400 nmol/L concentrations of each of the appropriate primers, and 2 μL of template DNA in a final volume of 25 μL. An iCycler or MyiQ thermocycler (Bio-Rad) was used for all reactions with the following program: 95° C. for 3 min, followed by 40 cycles of 15 s at 95° C., 15 s at the appropriate annealing temperature, and 15 s at 72° C. A final melt at 95° C. for 1 min was done prior to a dissociation curve analysis (55° C. to 95° C. in 0.5° C. steps for 10 s increments). Fluorescence signals were measured every cycle at the end of the annealing step and continuously during the dissociation curve analysis. The resulting data were analyzed using iQ5 optical system software (Bio-Rad). All reactions were performed in duplicate (within the assay). A standard curve consisting of a 10-fold dilution series of plasmid containing the target DNA sequence was included with each set of samples analysed. Results were reported as target copy number per gram of tissue or feces.

### Exemplary Recipes for BAM BJ and CVS

### Exemplary BJ Agar

Trypticase soy agar 40 g

Pig Feces Extract 50 ml

Sterile distilled water 976 ml

Colistin—3.125 μg/ml-12.5 μg/ml (e.g. 6.25 μg/ml) concentration in final plate

Vancomycin—3.125 μg/ml-12.5 μg/ml (e.g. 6.25 μg/ml) concentration in final plate

Spiramycin—12 μg/ml-50 μg/ml (e.g. 25 μg/ml) concentration in final plate

Rifampicin—6.25 μg/ml-25 μg/ml (e.g. 12.5 μg/ml) concentration in final plate

Spectinomycin—50 μg/ml-400 μg/ml (e.g. 200 μg/ml) concentration in final plate

Bovine blood 50 ml

### Modified BJ Agar

Trypticase soy agar 30-50 g

Pig Feces Extract 30-70 ml

Sterile distilled water 900-976 ml (modified according to volume changes)

Colistin—3.125 μg/ml-12.5 μg/ml (e.g. 6.25 μg/ml) concentration in final plate

Vancomycin—3.125 μg/ml-12.5 μg/ml (e.g. 6.25 μg/ml) concentration in final plate

Spiramycin—12 μg/ml-50 μg/ml (e.g. 25 μg/ml) concentration in final plate

Rifampicin—6.25 μg/ml-25 μg/ml (e.g. 12.5 μg/ml) concentration in final plate

Spectinomycin—50 μg/ml-400 μg/ml (e.g. 200 μg/ml) concentration in final plate

Bovine blood 40-60 ml

Pig feces extract can be made according methods known in the art for example (Kunkle R. A., Harris D. L., Kinyon J. M. Autoclaved liquid media for propagation of Treponema hyodysenteriae. J. Clin. Micro. October 1998, p 669-671). The procedure described below is modified in that the extract is diluted 1:1 with PBS prior to freezing. For example pig feces extract can be prepared according to the following:

### 1 Part Pig Feces (200 g): 4 Parts PBS (800 ml)

The Feces+PBS is agitated, for example by placing in a flask with a stir bar, and stirred, for example, overnight in a fridge. After a suitable length of time, for example, the next day, the stir plate is turned off and the contents are allowed to settle. The mixture is centrifuged to separate the solid and liquid fractions. The supernatant is decanted into a new flask and mixed again with equal parts PBS (˜1 L). Aliquots of the desired volume are frozen for use later. Feces should ideally be obtained from a healthy, young, growing pig that has not been treated with antibiotics. Solid stools should be used.

### Exemplary CVS Agar

Trypticase soy agar 20 g

Sterile distilled water 438 ml

Colistin—3.125 μg/ml-12.5 μg/ml (e.g. 6.25 μg/ml) concentration in final plate

Vancomycin—3.125 μg/ml-12.5 μg/ml (e.g. 6.25 μg/ml) concentration in final plate

Spectinomycin—50 μg/ml-400 μg/ml (e.g. 200 μg/ml) concentration in final plate

Bovine blood 50 ml

### Modified CVS Agar

Trypticase soy agar 15-25 g

Sterile distilled water 418 to 438 ml (according to volume changes)

Colistin—3.125 μg/ml-12.5 μg/ml (e.g. 6.25 μg/ml) concentration in final plate

Vancomycin—3.125 μg/ml-12.5 μg/ml (e.g. 6.25 μg/ml) concentration in final plate

Spectinomycin—50 μg/ml-400 μg/ml (e.g. 200 μg/ml) concentration in final plate

Bovine blood 50 ml 40-60 ml

The agar is poured into petri plates for example in 10 mm (height), or standard 15 mm plates (height). Other plate sizes can also used. Thinner plates for example have been found to save space in anaerobic growth jars.

Growth of Brachyspira sp. Sask30446 on BAM

Brachyspira sp. Sask30446 is a fastidious, slow growing, anaerobe. Growth of Sask30446 on solid medium has best been achieved on BAM-SR agar (Blood Agar Base no. 2 (Oxoid) (40 g l⁻¹), Beef Extract (Difco) (3 g l⁻¹) and Bacto Peptone (Difco) (5 g 1′)), supplemented with defibrinated horse blood (7%), spectinomycin (400 mg ml⁻¹) and rifampin (30 mg ml⁻¹) (Calderaro et al., 2005). Growth (confirmed by a positive PCR result with a Sask30446-specific assay) and Gram stain, has also been observed on this media made with sheep blood instead of horse blood, although isolated colonies were not obtained.

Direct inoculation of agar media with colon tissue from an affected pig can be used.

Inoculation of agar media through a 0.45 μm filter (sample incubated on top of filter on top of media) can be used.

Inoculation of agar media with broth that was incubated with the tissue sample at room temperature for 30 minutes can be used. Inoculation can be directly on to the agar surface or through a 0.45 micron filter.

After 8 days of incubation at 37° C., Brachyspira sp. Sask30446 is visible as a hemolytic (on horse blood), tiny, clear, wet/glistening, “fried egg” shaped colony. The colonies are less than 1 mm in diameter (FIG. 1).

Gram stains of isolates from agar plates show pleiomorphic, Gram negative spirochetes with tapered ends. Length of the cells is variable, 2-20 μm (FIG. 2). Spirochetes with the same morphology are abundant in samples of feces from pigs with hemorrhagic colitis that also test positive by the Brachyspira sp. Sask30446 qPCR assay (primers JH0224/JH0225) with counts of ˜1×10⁷ Brachyspira sp. Sask30446 organisms per gram of feces or colon contents (FIG. 3).

One or more colonies are isolated by extracting the colony and/or hemolytic zone and optionally subculturing onto a solid media such as BJ or CVS.

Growing Brachyspira Sp. Sask30446 on BJ or CVS

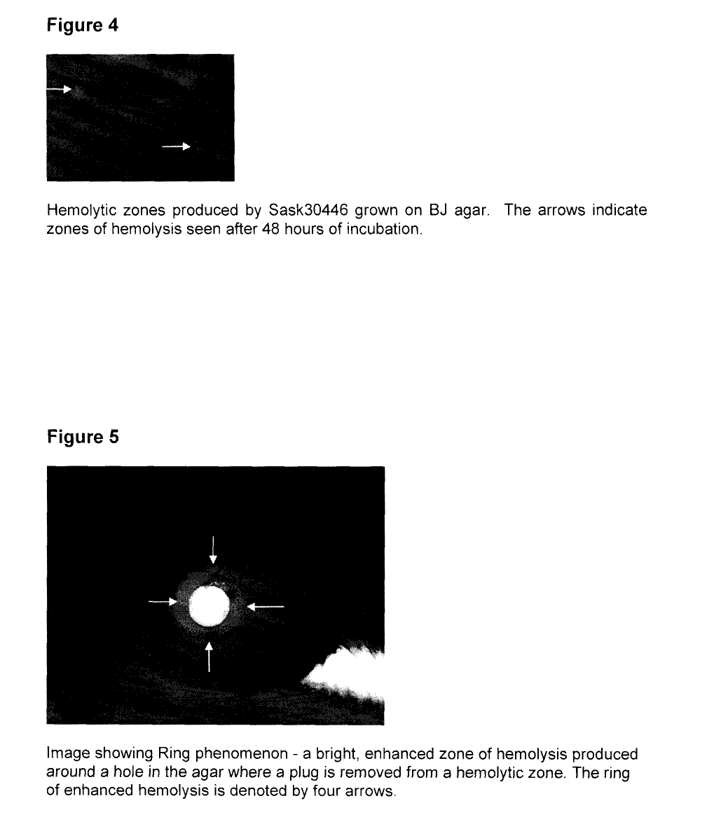

Brachyspira sp. Sask30446 can be grown on solid media such as BJ or CVS. For example, a sample is obtained that comprises or is suspected of comprising Sask30446 organisms (such as a fecal swab) and a BJ or CVS agar plate is inoculated. The plate is grown at about 42° C. under anaerobic conditions in an anaerobic jar with Oxoid anaerobic gas pack. Very good growth is seen after about 48 hrs (FIG. 4).

Growth is indicated by zones of hemolysis. At 48 hours the anaerobic jar is opened and hemolytic zones are sub-cultured to fresh media—because no colonies are formed, multiple subcultures (for example 4) are used to obtain a pure culture. For each sub-culture a single round zone of hemolysis is subcultured on to fresh media and incubated as previously described.

On BJ and CVS agar media, a bright, enhanced zone of hemolysis is produced around a hole in the agar where a plug is removed from a hemolytic zone (FIG. 5). Note approximately the right 23 of the image is hemolytic, the ring is on the edge of the hemolytic zone.

### Dark Field Microscopy.

Brachyspira sp. Sask30446 (and other spirochetes) can be visualized from either clinical samples, or cultures in/on media using dark field microscopy or hanging drop microscopy. The snake-like morphology of the organism can be seen, as well as its characteristic motility. Dark field microscopy is a useful technique for visualizing the spirochetes.

### Broth Culture

Brachyspira sp. Sask30446 can be grown and/or expanded by broth culture using a method described herein. For example, a pure culture of Brachyspira sp. Sask30446 is grown up on solid media (e.g. BJ or CVS) for about 48 hours. The solid media can serve as an inoculum to inoculate a broth culture comprising BHI and/or HI, 1-20% blood product (e.g. preferably ovine and fetal calf serum) and 0.5 to 10% glucose (e.g. preferably 1% glucose v/v).

For example, a section of agar (e.g. confluent hemolysis to ensure maximal bacterial numbers) is used to inoculate broth. For example, the agar section used to inoculate the broth can be approximately 1-2 cm². Other sizes can also be used. The agar section can also for example be a top slice (e.g. top half of agar thickness), or a slice through the thickness of an agar plate (e.g. 100×100 mm plate).

The agar slice is put into for example a vial containing 10 ml JBS broth and optionally a magnetic stir bar.

For example, JBS broth contains the following:
i. 9 ml BHI (brain heart infusion) (Becton Dickinson, Sparks Md.) broth supplemented with about 1% glucose
ii. 0.5 ml deactivated fetal calf serum
iii. 0.5 ml ovine blood

JBS broth can be made according to the following:
i. Brain Heart Infusion (BHI) broth+1% glucose is prepared and for example 9 ml is aliquoted into vials (for example into 32 ml vials) which contain a clean magnetic stir bar.
ii. The vials containing the BHI broth+glucose and stir bar are autoclaved to sterilize the media.
iii. In a biosafety cabinet (laminar air flow to reduce the risk of contaminating the sterile media) 0.5 ml of deactivated fetal calf serum and 0.5 ml of ovine blood are added.

The vial lid is loosely attached allowing the anaerobic atmosphere to reach the media.

The vial is placed inside an anaerobic jar, a gas pack (such as oxoid packs described previously) and optionally an anaerobic indicator strip are inserted and the jar is sealed.

The jar is then incubated at for example 39° C. on a stir plate.

After approximately 24 hours of incubation, large numbers of spirochetes can be seen using dark field microscopy.

The spirochetes can be isolated for example by spinning down the broth culture. The isolated spirochetes can for example be resuspended in a suitable diluent and/or freezing solution and frozen for example at −80° C.

A culture of the spirochetes can also be frozen for example at −80° C. The culture can be thawed and grown for example at 39° C.

Additional Brachyspira sp. Sask30466 sequences

Brachyspira sp. Sask30446 Bnox (810 nucleotides)
(SEQ ID NO: 9)
CCTGAAGGTTTGAAATCTGAAGGCATCGATGTTTATATGGGACATGAAGT
AACAAAAATAGACTGGGCTAACAAAAAATTACATATCAAAGAATTAAAAA
CAGGTAAAGAGTTTGAAGACAATTATGATAAACTTATACTTGCTACTGGT
TCTTGGCCTGTAACTCCTCCTATAGAAGGATTAAAACAAGAAGGTACTGA
ATACGGTCTTAAAAAAGGTATTTTCTTCGCTAAATTATTCCAACAAGGAC
AGGATATCATTAATGAAATAGCTAAACCTGAAGTAAAAAAAGTTATGGTT
GTTGGTGCTGGTTATATAGGTGTTGAGCTTATAGAAGCATTCAAAAACCA
TGGTAAAGAAGTTATCTTAATGGAATCTATGCCTAGAGTTATGGCTAACT
ATTTTGATAAAGAAATCACTGATGAAGCTGAAAAAAGAATCATAGATGCT
GGAATAGAAATGCGTTTAGGTGAAACTGTTAAAAAGTTTGAAGGTGATGA
CAGAGTTAAAAAAGTTGTTACCGATAAAGGTTCTTATGATGTAGATATGG
TAGTTATGTCTGTTGGTTTCAGACCTAATAGCGAACTTTATAAAGACTAT
TTGGAAACTTTACCTAACGGTGCTATTGTAGTAGATACTACTATGAGATC
AAGCAAAGATCCTGATGTTTATGCTATAGGAGACTGTGCTACTGTATATT
CAAGAGCTTCTGAAAAACAAGAGTATATTGCTTTAGCTACTAATGCTGTA
AGAATGGGTATTGTTGCTGCTAATAATGCTTTAGGAAAACATGTTGAATA
CTGCGGTACT
Brachyspira sp. Sask30446 Nox amino acid sequence
is 92.2% identical to its nearest DNA relative:
EF517546 Brachyspira innocens isolate C336
(SEQ ID NO: 10)
CCTGAAAGTTTAAAAGCTGAAGGCATAGATGTTTATATGGGACATGATGT
AACAAAAATAGACTGGGCTAATAAAAAATTACATGTTAAAGAATTAAAAA
CAGGTAAAGAGTTTGATGATAATTATGACAAACTTATTCTTGCTACAGGT
TCTTGGCCTGTAACTCCTCCTATAGAAGGTTTAATGCAGGAAGGTACTGA
ATACGGACTTAAAAAAGGTATTTTCTTCTCTAAATTATTCCAGCAAGGAC
AAGAAATTATTGATGAAATAGCTAAACCTGAAGTAAAAAAAGTTATGGTA
GTTGGTGCTGGTTATATAGGTGTTGAACTTATAGAAGCTTTCAAAAATCA
TGGTAAAGAAGTTATATTAATGGAAGCTATGCCTAGAGTTATGGCTAACT
ATTTTGACAAAGAAATTACTGATGAAGCTGAAAAAAGAATCAAAGAAGCT
GGCATAGAAATGCATTTAGGTGAAACTGTTAAAAAATTTGAAGGTGATGA
CAGAGTTAAAAAAGTTATTACTGACAAAGGTTCTTATGATGTAGATATGG
TAGTTATGTCTGTTGGTTTCAGACCTAATAGTGAGCTTTATAAAGATTAT
TTAGAAACTTTACCTAATGGTGCTATAAAAGTAGACACTACTATGAAAAC
TACAAAAGATCCTAATGTATTTGCTATAGGAGACTGTGCTACTGTATATT
CAAGAGCTTCTGGAAAAGAAGAATACATCGCTTTAGCTACTAATGCTGTA
AGAATGGGA
TTGTTGCTGCTAACAATGCTTTAGGAAAACATGTTGAATACTGCGGTACT
>translation of 241 bp fragment from Brachyspira sp.
Sask30446
(SEQ ID NO: 11)
AKFIQQGQDIINEIAKPEVKKVMVVGAGYIGVELIEAFKNHGKEVILMES
MPRVMANYFDKEITDEAEKRIIDAGIEMR
>810 bp translation from Brachyspira sp. Sask30446
SEQ ID NO: 12
PEGLKSEGIDVYMGHEVTKIDWANKKLHIKELKTGKEFEDNYDKLILATG
SWPVTPPIEGLKQEGTEYGLKKGIFFAKLFQQGQDIINEIAKPEVKKVMV
VGAGYIGVELIEAFKNHGKEVILMESMPRVMANYFDKEITDEAEKRIIDA
GIEMRLGETVKKFEGDDRVKKVVTDKGSYDVDMVVMSVGFRPNSELYKDY
LETLPNGAIVVDTTMRSSKDPDVYAIGDCATVYSRASEKQEYIALATNAV
RMGIVAANNALGKHVEYCGT
>AAC78822 NADH oxidase [Brachyspira murdochii
DSM 2563]
(SEQ ID NO: 13)
PESLKAEGIDVYMGHDVTKIDWANKKLHVKELKTGKEFDDNYDKLILATG
SWPVTPPIEGLMQEGTEYGLKKGIFFSKLFQQGQEIIDEIAKPEVKKVMV
VGAGYIGVELIEAFKNHGKEVILMEAMPRVMANYFDKEITDEAEKRIKEA
GIEMHLGETVKKFEGDDRVKRVVTDKGSYDVDMVVMSVGFRPNSELYKDY
LETLPNGAIKVDTTMKTTKDPNVFAIGDCATVYSRASGKEEYIALATNAV
RMGIVAANNALGKHVEYCGT

Brachyspira murdochii and B. innocens are the species with the closest related sequence to the translation product of the 810 Brachyspira sp. Sask30446 nox1 sequence and are 91% identical (SEQ ID NO:13) and 92-93% identical respectively, depending on the particular strains of each species being compared.

>AAC78816 NADH oxidase [Brachyspira pilosicoli]
(SEQ ID NO: 14)
SKLYQQGQDIINEIAKPEVKKVMVVGAGYIGVELIEAFKNHGKEVILMEA
MPRVMANYFDKEITDEAEKRIKDAGIELH

The above sequence, from Brachyspira pilosicoli, is 91% identical to B. sp. Sask30446 NADPH oxidase at the amino acid level, which is the closest reference sequence for this region of amino acid sequence.

In addition, the DNA sequences of several other genes in the genome of Brachyspira sp. Sask30446 have been determined. All sequences presented here were derived from cultured isolates and based on currently available sequence data for Brachyspira spp.

Sequences for chaperonin-60 and 16S rRNA were determined as described below, using established primers and PCR protocols to amplify the target regions from isolates.

A Multi Locus Sequence Typing (MLST) scheme originally published for the identification and strain differentiation of Brachyspira spp. by Rasback et al. (2007b) was applied to isolates of Brachyspira sp. Sask30446. Six samples from three separate culture plates were examined. In all cases, identical sequences were obtained for each of the targets. Representative sequences for each target are included, along with protein translations of the relevant open reading frames.

TABLE 1
Primers used in MLST (Rasback et al., 2007b)

| | PRIMER | |
|---|---|---|
| TARGET | NAME | SEQUENCE (5′-3′) |
| esterase (est) | EST-F229 | GATGCTTCAGGCGGAGTTATG (SEQ ID NO: 15) |
| | EST-R847 | CCACACTCATAGCATAAATACTG (SEQ ID NO: 16) |
| glucose kinase | GLPK-F123 | AGGCTGGGTAGAACATAATGC (SEQ ID NO: 17) |
| (glpk) | GLPK-R1158 | TCTTTACTTTGATAAGCAATAGC (SEQ ID NO: 18) |
| phosphoglucomutase | PGM-F172 | GTTGGTACTAACAGAATGAATA (SEQ ID NO: 19) |
| (pgm) | PGM-R1220 | CCGTCTTTATCGCGTACATT (SEQ ID NO: 20) |
| acetyl-CoA | THIO-F163 | TGTGTTATACAATCAGCACTTC (SEQ ID NO: 21) |
| acetyltransferase | THIO-R1079 | GTAGTAAGTATTCTAGCTCCAG (SEQ ID NO: 22) |
| (thioloase)(thi) | | |

### Chaperonin-60 (Cpn60 or GroEL or Hsp60)

Target sequence corresponds to the cpn60 “universal target”, widely exploited for bacterial species identification (Hill et al., 2004) (e.g. universal, degenerate PCR primers which can be applied for the amplification of a 549-567 bp region of cpn60 corresponding to nucleotides 274-828 of the E. coli cpn60 sequence from virtually any genome). Sequence was amplified from an isolated colony using primers H729 (5′-CGC CAG GGT TTT CCC AGT CAC GAC GAI III GCI GGI GAY GGI ACI ACI AC-3′) (SEQ ID NO:23) and H730 (5-AGC GGA TAA CM TTT CAC ACA GGA YKI YKI TCI CCR MI CCI GGI GCY TT-3′) (SEQ ID NO:24) and published PCR conditions (Brousseau et al., 2001).

>v12564 Brachyspira sp. Sask30446 chaperonin-60
(cpn60)
(SEQ ID NO: 25)
GCTACTGTTTTAGCTCAGGCTATGGTTAAAGAAGGTTTGAAAAATGTAA
CTAGCGGTGCTAATCCTATGCTTATTAAAAGAGGTATAGAAAAAGCTGT
TAGCGAAATAGTTGCTTATATCAAATCTGAAGCTAAACAAATTAAAGGC
AAAGAAGAAATTGCTCAGGTTGCTACTATTTCTGCTAACAATGATAAAG
AAATTGGTGCTTTAATCAGTGATGCTATGGAAAAAGTTGGTAAAGAAGG
CGTTATCACTGTAGAAGAAGCTAAATCTTTAGAAACTAGTCTTTCTTTG
GTAGAAGGTATGCAGTTTGACAGAGGTTATATCTCTCCATATTTCGTAA
CTAACGGAGATAGTATGACTGCTGAATTAGAAGATGCTTTACTTCTTAT
CTATGATAAAAAAATCTCTAACATGAAAGAACTTCTTCCTATACTTGAA
AAAATTGCTCAGACTGGAAGACCTTTCATTATTATCGCTGAAGATATTG
AAAATGAAGCTTTGGCTACTTTGGTACTTAACAAAATGAGAGGAGTATT
AAATGTATGTGCTGTT

This nucleotide sequence is 97% identical to Brachyspira intermedia(Genbank Accession JF907595), 96% identical to Brachyspira murdochii DSM 12563 (Genbank Accession CP001959), 96% identical to Brachyspira murdochii ATCC 51284 (Genbank Accession DQ099908), 95% identical to Brachyspira innocens ATCC 29796 (Genbank Accession DQ099906), 94% identical to Brachyspira hyodysenteriae WA1 (Genbank Accession CP001357), 94% identical to Brachyspira hyodysenteriae ATCC 27164 (Genbank Accession DQ099905), 92% identical to Brachyspira pilosicoli ATCC 51139 (Genbank Accession DQ099903), 91% identical to Brachyspira alvinipulli ATCC 51933 (Genbank Accession DQ099907), and 84% identical to Brachyspira aalborgi ATCC 43994 (Genbank Accession DQ099904). The nucleotide sequence encodes the following peptide sequence (reading frame+1):

>v12564 Brachyspira sp. Sask30446 chaperonin-60
(cpn60)
(SEQ ID NO: 26)
ATVLAQAMVKEGLKNVTSGANPMLIKRGIEKAVSEIVAYIKSEAKQIKGK
EEIAQVATISANNDKEIGALISDAMEKVGKEGVITVEEAKSLETSLSLVE
GMQEDRGYISPYFVTNGDSMTAELEDALLLIYDKKISNMKELLPILEKIA
QTGRPPIIIAEDIENEALATLVLNKMRGVLNVCAV

The nearest peptide neighbour is B. hyodysenteriae at 98% sequence identity. A phylogenetic tree showing the relationship of Brachyspira sp. Sask30446 to other species, based on cpn60 universal target sequence (555 bp) is shown in FIG. 4.

### Esterase (Est)

DNA sequence of Brachyspira sp. Sask30446 est MLST target:

>Brachyspira sp. Sask30446 esterase (est)
(SEQ ID NO: 27)
AAATATTTAGAAGAGAGAAATATAGGTTTTGATGTTGGAGTTAC
AAAGGTTCCTTTGGTTTGTCAGTCTTGTATATTTGATTTGCGTG
TTGGGGATTATAAAGTTCGTCCGGATATTAATATGGCTTATGAG
GCTTGTATCAATGCTCAAAATAATAATCCAAAAATGGGAAATTA
TGGAGCTGGTACAGGTGCTAGTGTTGGAAAAATACTTGGTGCTG
ATTATACTATGAAGTCTGGGCTTGGTTTTTATGCTGTTCAAATT
GATGATGTTAAGGTTGGTGCTATAGTTGCTCTTAATGCTTTCGG
CGATATTTATGATTATGATAATGGAAAAATGATTGCTGGTCTTC
TTAATGAAAATAAAGATGGGTTTAGAAGTTCTGAAGAGGAGCTT
ATAAAAATAACGCAGAATAATAATTTGTCTTTTACTTCTAATAA
TAATATAGTTACAAATACTACAATAGGAGCTGTAATTACAAATG
CCAAGTTTACAAAGTCACAAATGGGAAAAATAGCTTCTATGGCA
CATAACGGCTTTGCTAGGGCTATAAAACCAGTGCATACTACAGT
AGATGGCGA

This DNA sequence is 93% identical to the est sequence of Brachyspira murdochii DSM12563 (Genbank Accession CP001959), 86% identical to Brachyspira hyodysenteriae WA1 (Genbank Accession CP001357). This nucleotide sequence encodes the following protein sequence (Reading frame+1):

>Brachyspira sp. Sask30446 esterase (est)
(SEQ ID NO: 28)
KYLEERNIGFDVGVTKVPLVCQSCIFDLRVGDYKVRPDINMAYE
ACINAQNNNPKMGNYGAGTGASVGKILGADYTMKSGLGFYAVQI
DDVKVGAIVALNAFGDIYDYDNGKMIAGLLNENKDGFRSSEEEL
IKITQNNNLSFTSNNNIVTNTTIGAVITNAKFTKSQMGKIASMA
HNGFARMKPVHTTVDG

The nearest peptide neighbour is B. murdochii at 95% sequence identity.

### Glucose Kinase (Glpk)

DNA sequence of Brachyspira sp. Sask30446 glpk MLST target:

>Brachyspira sp. Sask30446 glucose kinase
(glpk)
(SEQ ID NO: 29)
GGAGTTTGCAGGAGCTATACAGATTGCTGGAGTTAAGCCTGAAG
AGATAGCTGCTATTGGTATTACAAATCAAAGGGAAACTACTGTT
GTATGGGATAAAAATACTGGAGAGCCTATTTATAATGCTATAGT
ATGGCAATGTAGAAGAACTGCTCCTATTTGCGATGATTTAAAGA
AAAAAGGACTTGATACTTATATAAGAGAAAATACAGGTTTAGTT
GTTGATGCTTATTTTTCTGGTACAAAAATAAAATGGATACTTGA
TAATGTTCCTGGTGCTAGAGAAAAAGCCAATAAAGGAGAATTAC
TATTTGGTACAATAGACACTTGGCTTGTATGGAAACTTACAGGC
GGTAAAGTTCATGTTACAGACTATACTAATGCATCAAGGACTAT
GATTTATAATATCAAAGATTTAAAATGGGACGAAAACATTTTAA
GAGAATTAGACATTCCTATGAGCATGCTTCCAGAGGTAAAAGAT
TCTTCTTGTGTTTACGGATATGCTAATATTAACGGTAAAGAAGT
ACCTATATCTGGAATAGCAGGAGACCAGCAGTCAGCATTATTCG
GTCAGGCTGGATTTAATAAGGGCGACACAAAAAATACTTATGGT
ACTGGAAGTTTCATTCTTATGAATATAGGAGAGAATTTTATATT
AAGTAAAAACGGACTTATTACTACTATAGGTATAGGATATAATG
GAAAAATAGAATATGCTTTGGAAGGTTCTGTATTTATAGCTGGT
GCTGTTATACAATGGGTACGCGATGAATTAAGACTTCTTCATGA
TGCAAAAGACACTGAATATTTCGCTACTAAAGTAAAAGATACTA
ACGGAGTTTATTTAGTACCTGCATTTGTTGGTCTTGGTGCTCCT
TATTGGGATATGTATGCTAGAGGCTGTTTAGTAGGTATTACAAG
AGGGGT

This sequence is 95% identical to Brachyspira murdochii DSM 12563 (Genbank Accession CP001959), 90% identical to Brachyspira hyodysenteriae WA1 (Genbank Accession CP001357) and 89% identical to Brachyspira pilosicoli strain 951000 (Genbank Accession CP002025). The nucleotide sequence encodes the following protein sequence (reading frame+2):

>Brachyspira sp. Sask30446 glucose kinase (glpk)
(SEQ ID NO: 30)
EFAGAIQIAGVKPEEIAAIGITNQRETTVVWDKNTGEPIYNAIVWQ
CRRTAPICDDLKKKGLDTYIRENTGLVVDAYFSGTKIKWILDNVPG
AREKANKGELLFGTIDTWLVWKLTGGKVHVTDYTNASRTMIYNIKD
LKWDENILRELDIPMSMLPEVKDSSCVYGYANINGKEVPISGIAGD
QQSALFGQAGFNKGDTKNTYGTGSFILMNIGENFILSKNGLITTIG
IGYNGKIEYALEGSVFIAGAVIQWVRDELRLLHDAKDTEYFATKVK
DTNGVYLVPAFVGLGAPYWDMYARGCLVGITRGV

The nearest peptide neighbour is B. murdochii at 99% sequence identity.

### Phosphoglucomutase (Pgm)

DNA sequence of Brachyspira sp. Sask30446 pgm MLST target:

>Brachyspira sp. Sask30446 phosphoglucomutase
(pgm)
(SEQ ID NO: 31)
CAGGTTTAGCTAATTATATATTAAAACAAGGCGGAAGTGATTATA
AAGTTGCTATAGGATATGATTCAAGAAATAATTCTGATGTATTTT
CAAAAGCTGCTGCTGAGATACTTTCTTCAAACGGAATAAAAGTTT
ATTTATATGATGATATTCACCCTATTTCACTTCTTTCTTATGCTG
TTAGAAGTTTAGGCTGTATTGCAGGAATAGTTGTTACTGCTAGTC
ATAACCCTAAGGAATATAATGGATATAAAGTTTATTGGACTGACG
GAGCTCAGGTTATACCGCCTCATGACAAAAATATCATAGATGAAG
TATTAAAAGTTAAGCCTGAAGAAGTAAAAATGGGCGACTCTTCAA
AAATTACTATAATAGGAAAAGATATTGAAGATAAATACATGAATG
ATTTGATGGTATATTTAGTTAATCCAGACATCATTAAAAAACATC
ATGATATAAAAATAGTTTATACCCCAATTCATGGTTCTGGATACA
AAATGGTTCCTATGGCTTTAAGAAAGGCTGGTTTTACAAATTTAA
CAACATTAGAAGGTGCTCAGCCTCCAGACGGAAATTTTCCTACTG
TAGAATCTCCTAACCCAGAAAATCCTGAAGCATTGCAGATAGCCG
TTAATAAAGCTAAAGAGATAGGTGCAGAACTTGTTATGGGTACCG
ACCCAGACTGCGACAGAATGGGATGTGCTTTGCTTACTAAAGATG
GAAGCTATATGTATCTTACAGGTAATCAGATAGGATCCATAATGG
CATACTATCTCATCACAAACAAAAAAAATATTAAAAATCCGTACA
TAGTAAAAACAATAGTAACAACTGAACTAGCTAGGGCTATTGCTG
ATGCTAATAATGTTAAGATTTACGATGTACTTACAGGTTTTAAAT
GGATTGCTGATGTTATAGAAAGAGATAAAGAAGGTACATATTTAT

This sequence is 93% identical to Brachyspira murdochii DSM 12563 (Genbank Accession CP001959), 89% identical to Brachyspira hyodysenteriae WA1 (Genbank Accession CP001357) and 88% identical to Brachyspira pilosicoli strain 951000 (Genbank Accession CP002025). This nucleotide sequence encodes the following protein sequence (reading frame+3):

>Brachyspira sp. Sask30446 phosphoglucomutase
(pgm)
(SEQ ID NO: 32)
GLANYILKQGGSDYKVAIGYDSRNNSDVFSKAAAEILSSNGIKVYL
YDDIHPISLLSYAVRSLGCIAGIVVTASHNPKEYNGYKVYWTDGAQ
VIPPHDKNIIDEVLKVKPEEVKMGDSSKITIIGKDIEDKYMNDLMV
YLVNPDIIKKHHDIKIVYTPIHGSGYKMVPMALRKAGFTNLTTLEG
AQPPDGNFPTVESPNPENPEALQIAVNKAKEIGAELVMGTDPDCDR
MGCALLTKDGSYMYLTGNQIGSIMAYYLITNKKNIKNPYIVKTIVT
TELARAIADANNVKIYDVLTGFKWIADVIERDKEGTYL

The nearest peptide neighbour is B. murdochii at 99% sequence identity.

### Acetyl-CoA Acetyltransferase (Thioloase)(thi)

DNA sequence of Sask30446 thi MLST target:

>Brachyspira sp. Sask30446 acetyl-CoA
acetyltransferase (thioloase)(thi)
(SEQ ID NO: 33)
TATTAATGCCGGAATACCTGTAGACAAACCGGCATTAACTATAAAT
ATATTATGCGGTTCAGGATTAAGAGCTGTATCAATGGCAGCACAAA
TGATTAAATCAGGAGATGCTGATATAGTAGTTGCAGGCGGTACTGA
AAATATGAGTATGGCTCCATATACTTCTTCAGCTATGAGAATGGGT
GCTAGAATGGGCGAAAGTAAAATGCAGGATACTTTACTTAATGATG
CTCTTATTTGTGCTTTTGAGCATTATCATATGGGAGTTACTGCTGA
AAATGTTGCAGAACAATGGGGTATTACAAGACAAGAACAAGATGAG
TTTGCATGCAGAAGCCAGAACAGAGCAGAAGAAGCAGTAAAAAGCG
GAAGATTTAAAGATGAAATAGTACCTGTTACAATCAAAACTAGAAA
AGGCGAAATAGTAGTTGATACAGATGAACACCCTACTTTTGGTACT
ACTATGGAATCTTTAGCTAAATTAAAACCTGCATTCAAAAAAGATG
GTACTGTTACTGCTGGTAATGCTTCTGGTATCAATGATGCTGCTTC
AGCTGTAGTTATTATGTCTAAAGAAAAAGCTGATGAGCTTGGAATT
AAACCTATGGCTAAGATTTTAGGTTATGCTACTCATGGTGTTGAGC
CTAGAATAATGGGTATAGGACCTATAGAGGCAACTAGAAAGGCTTT
GAAAATGGCTAATCTTACAGTTGAAGATATGGACTTAATAGAAAGT
AATGAGGCTTTCGCTGCTCAGTCTATAGCTGTTGCCCGTGAGTTAA
AATTCAATATGGACATAGTTAATGTCAATGGCGGAGCTA

This nucleotide sequence is 88% identical to Brachyspira murdochii DSM 12563 (Genbank Accession CP001959), 92% identical to Brachyspira hyodysenteriae WA1 (Genbank Accession CP001357) and 88% identical to Brachyspira pilosicoli strain 951000 (Genbank Accession CP002025). This sequence encodes the protein sequence (reading frame+2):

>Brachyspira sp. Sask30446 acetyl-CoA
acetyltransferase (thioloase) (thi)
(SEQ ID NO: 34)
INAGIPVDKPALTINILCGSGLRAVSMAAQMIKSGDADIVVAGGTENMSMA
PYTSSAMRMGARMGESKMQDTLLNDALICAFEHYHMGVTAENVAEQWGITR
QEQDEFACRSQNRAEEAVKSGRFKDEIVPVTIKTRKGEIVVDTDEHPTFGT
TMESLAKLKPAFKKDGTVTAGNASGINDAASAVVIMSKEKADELGIKPMAK
ILGYATHGVEPRIMGIGPIEATRKALKMANLTVEDMDLIESNEAFAAQSIA
VARELKFNMDIVNVNGGA

The nearest neighbour is B. pilosicoli at 95% sequence identity.

Small Subunit Ribosomal RNA (16S rRNA or SSU RNA)

Amplified sequence corresponds to nucleotides 11-536 of the E. coli 16S rRNA gene (encompassing variable regions V1, V2 and V3). Amplification primers were H1476 (5′-GAG TTT GAT CCT GGC TCA G-3′) (SEQ ID NO: 35) and H1478 (5′-GWA TTA CCG CGG CKG CTG-3′) (SEQ ID NO:36) (Dorsch and Stackebrandt, 1992).

>Brachyspira sp. Sask30446 16S rRNA, partial
(SEQ ID NO: 37)
AGCGAACGTTGGCGATGCGTCTTAAGCATGCAAGTCGAGCGGACTTATTC
GGGCAACTGGATAAGTTAGCGGCGAACTGGTGAGTAACACGTAGGTAATC
TGCCGTAGAGTGGGGGATAACCCATGGAAACATGGACTAATACCGCATAT
ACTCTTACTACACAAGTAGAGTAGAGGAAAGGAGCAATCCGCTTTACGAT
GAGCCTGCGGCCTATTAGCCTGTTGGTGGGGTAACGGCCTACCAAAGCTA
CGATAGGTAGCCGACCTGAGAGGGTGACCGGCCACATTGGGACTGAGATA
CGGCCCAGACTCCTACGGGAGGCAGCAGCTGAGAATCTTCCACAATGGAC
GAAAGTCTGATGGAGCGACATCGCGTGAGGGATGAAGGCCTTCGGGTTGT
AAACCTCGGAAATTATCGAAGAATGAGTGACAGTAGATAATGTAAGCCTC
GGCTAACTACGTGC

This sequence, while not identical to any published 16S rRNA sequences, is 98-99% identical to several other Brachyspira spp. However, it is well-established that 16S rRNA sequences are insufficient to discriminate Brachyspira spp. The 16S rRNA sequences are widely used to describe new bacterial species, and was determined as part of the description of Brachyspira sp. Sask30446.

The genome of Brachyspira sp. Sask30446 was sequenced and hemolysin polypeptide sequences were identified by comparing Brachyspira hyodysenteriae WA1 (ATCC 49526) annotated “hemolysin” polypeptides to predicted ORFs in Brachyspira sp. Sask30446. Four Brachyspira sp. Sask30446 sequences were identified and are presented in SEQ ID NO: 42, 43, 44 and 45. Predicted HLY1 is about 92% identical to Brachyspira hyodysenteriae peptide YP_{—}00272041; predicted HLY2 is about 95% identical to Brachyspira hyodysenteriae peptide YP_{—}002720787; predicted HLY3 is about 96% identical to Brachyspira hyodysenteriae YP_{—}002721411 (annotated as “hlyB”) and predicted HLY4 is about 94% identical to Brachyspira hyodysenteriae YP³_{—}002721605 (annotated as “hlyC”).

HLY1 is 92% identical to B. hyo peptide
YP_00272041
>HLY1 contig00731_1 length = 262128 numreads =
69597
(SEQ ID NO: 42)
MRLDEYVHSKGYTESRSKAQDIILAGGVEVNGVKVTSKAHKIKDTDNIEV
IQNIKYVSRAGQKLEKAFTEFGISVENKTCLDIGASTGGFTDCLLKHGAK
KVYALDVGHNQLVYKLRNDNRVISIEDFNAKDIKREMENDEIPSIVVSDV
SFISISKIAPIIFKELNNLEFWVTLIKPQFEAEKGEVSKGGIIRDDLLRE
KILNNAISRITEIGFKEVNRTVSPIKGAKGNIEYLAHFII
HLY2 is 95% identical to B. hyo peptide
YP_002720787
>HLY2 contig00727_1 length = 219037 numreads =
59508
(SEQ ID NO: 43)
MLSALFSGSETAYTSIDDVTLMRLVREKKIKEEDKKYWEKSSSMIPTLLV
GNNIVNISASSIITVFAVRLADILPNISTNLMVTISTATITILIIIFGEI
LPKVIMRVNAEKMMpyLLYFMKFCHFIFKPITFLMDKITTFIMNYFVPKR
LRDAEKRSALSSMDDITTIIHLGHKEGIIKEYTHEMLTGVIDFRNKTVEE
IMTPRVDMVCIEAETDVNEIIKLTVETGLSRFPVYEETVDHIIGIFHTRA
LFKEYVKGGGKLNKIKKKAIDYIMLPYFVPETKTISSLENDMQKKKLQMV
ITIDEYGGTAGLVTMEDITEEIMGDIEDESDKKEADVIRFKGKRIIINGN
APIEDVNKTLKLQLEHEEYQTIAGYVIDMLDHIPEINERFILKGYRVRIM
KVEDRRIVEMEFTPLKYTRTNENENTDTQETSDLEKNDLEILNE
HLY3 is 96% identical to B. hyo YP_002721411
(annotated as ″hlyB″)
>HLY3 contig00663_1 length = 151870 numreads =
47773
(SEQ ID NO: 44)
MFQFHLTSKAKKVIELYAQEEAKRLNHDMVTPEHILLGLLHESEALATRV
LLRLKIDLDRLKLELESAMVKSSTTKVEGTLPTAPRVQKLISRSAEEARA
LSHNYIGTEHLLLGLLREESGTAYNVLTSMGLELTILRQEILKMLGVAGS
NMSSMEQTNQEDTIKKVKTPTLDQFARDLTKMARDKALDKVIGRENEVMR
VVQILSRRKKNNPILLGEPGVGKTAIVEGLAEKIVAGDVPDILLKKRVLT
LDLSSVVAGTKYRGEFEERIKNIVLEIKKANNIIIFIDELHTLIGAGGAE
GALDAANMLKPALSRGEIQCIGATTINEYKKYIEKDGALVRRFQPINVEE
PSVEDTIEILNGIKPKYEEHHKVKYTDEAITAAAVLSKRYIFERHLPDKA
IDLIDEAGSRARLLNMTRPQEFKDLEKKIEELNQNKRNAVDNQNFEDAAR
IRDEISSLQEELSIKEEKWRGEREKIETFIEEDDIRHVISEITNIPIKRL
LNSESKRLIGMEEELHQKVVGQEEAISSISKAIRRSRAGLKTSKRPLGSF
IFLGPTGVGKTALAKVLSEFMFGDSDALIRIDMSEFMEKFAVSRLIGAPP
GYVGYEEGGGLTEKVRRKPYSLILFDEIEKAHPDVTNILLQVLEEGQLTD
NFGRKVDFSNTIIIITSNLGARDIVKGSSLGFNAVGSEKDANDIKNFALE
ELKQNFNPEFLNRIDDIIVFHTLSKDNLKDIINIMLKELNDAIKERNIVI
NLSEEAKNYIIDKGEDKKYGARSLRRAIQKEIEDYVSTEILFGNIEDGDT
INVDANDGSLIFSYDKSVRTENKELSKS
HLY4 is 94% identical to B. hyo YP_002721605
(annotated as ″hlyC″)
>HLY4 contig00657_1 length = 120161 numreads =
42457
(SEQ ID NO: 45)
MPIHKLISKILKKKDNNTDKNNYVNLSSLTEAEREIITNTIELKTKSVRE
IMVPRVDVVMIPIESSYDRVIKAFNKDRNSRIPVYKDGIDDIVGVLYVKD
LIDAEEKTFSLKKILHKPLFVPISISLMELLKNFREKQIHIAMVVDEYGG
FSGIVSMEDVLEQIIGDIRDEYDEEDEEIKSNDDGTYLVDARTRIDDENK
YDILPPIPDDEADTVGGELFSYLGRLPKRNEDIEYNGYSFTVVGKSGNIV
TKIRIE

B. sp. Sask30446 demonstrates strong hemolysis. The hemolysis genes for example may be associated with virulence. Accordingly in an embodiment, the isolated Brachyspira sp. Sask30446 organism comprises one or more sequences of SEQ ID NO: 42, 43, 44, and/or 45, for example comprising a polypeptide comprising one or more sequences of SEQ ID NO: 42, 43, 44, and/or 45 and/or a nucleic acid molecule encoding one or more sequences of SEQ ID NO: 42, 43, 44, and/or 45; or one or more sequences having at least 92.5% sequence identity with SEQ ID NO: 42, 95.5% sequence identity with SEQ ID NO:43; at least 96.5% sequence identity with SEQ ID NO:44 and/or 94.5% sequence identity with SEQ ID NO: 45 and/or any combination thereof.

In an embodiment, the method, kit, or composition comprises an isolated Brachyspira sp. Sask30446 organism comprising one or more sequences of SEQ ID NO: 42, 43, 44, and/or 45, for example comprising a polypeptide comprising one or more sequences of SEQ ID NO: 42, 43, 44, and/or 45 and/or a nucleic acid molecule encoding one or more sequences of SEQ ID NO: 42, 43, 44, and/or 45; or one or more sequences having at least 92.5% sequence identity with SEQ ID NO: 42, 95.5% sequence identity with SEQ ID NO:43; at least 96.5% sequence identity with SEQ ID NO:44 and/or 94.5% sequence identity with SEQ ID NO: 45 and/or any combination thereof.

### Immunogenic Compositions

An immunogenic composition is made for example by taking a sample from an animal confirmed to be infected with Brachyspira sp. Sask30446 and optionally growing on agar and/or in liquid broth as described herein. Other sources may be used such as previously isolated Brachyspira sp. Sask30446 including for example Brachyspira sp. Sask30446 strains with characteristics of the strain deposited with the International Depository of Canada (1015 Arlington St., Suite H3390 Winnipeg, MB R3E 3R2) on Nov. 16, 2011 under Accession number 16111-01 and/or a progeny or immunologically active derivative thereof. The sample is streaked on a solid agar plate and incubated for about 48 hrs. A section of the solid agar comprising a hemolytic zone is removed and placed in liquid media comprising ovine blood product. The inoculated liquid media is incubated for a sufficient time (for example 24 hours) and the Brachyspira sp. Sask30446 is isolated by spinning the culture media to obtain a pellet. The pellet is resuspended in a sterile diluent. The composition is suitably formulated for introduction into a subject such as an animal. The formulation is introduced into a subject and antibodies specific for the immunogenic composition are detected after a suitable number of days.

For example, rabbits were immunized with whole bacterial cells isolated from a broth culture, inactivated in 10% formalin repeatedly, for example at least 4 times, using a standard protocol and serum was isolated from each rabbit. An example of a standard protocol includes obtaining pre immune serum from the animal to be inoculated, immunizing with formalin inactivated whole cell culture in admixture with adjuvant such as Complete Freund's adjuvant (CFA), immunizing again at day 14 for example using CFA, immunizing further at days 28 and 35, for example using Incomplete Freund's Adjuvant (IFA), immunizing weekly thereafter for example for a total of 8 immunizations. The immunizations for example at days 42, 49, 56 and 63 comprised 0.9% NaCl. Further blood can be collected at for example the 7^{th} immunization to ELISA validate.

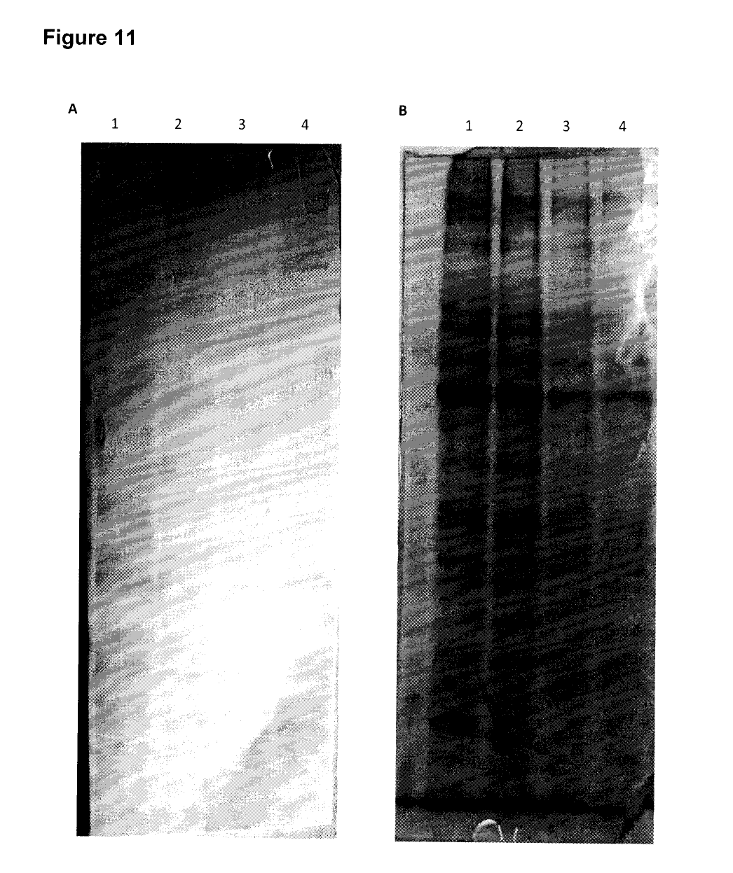

As shown in FIG. 11, antibodies raised recognize Brachyspira sp. Sask30446. FIG. 11 Panel A is a western blot showing specific immune detection of Brachyspira sp. Sask30446. The method used involved the following: Brachyspira sp. Sask30446 were grown in liquid culture and spun down for two minutes at 13,000 rpm. The pellets were washed twice in PBS. 50 μl of sample buffer (4.6% SDS, 10% Beta-mercaptoethanol, 20% glycerol, 1.5% Tris, 1% bromphenol blue) were added to the pellet. The samples were boiled for 2 min and stored at −80 degrees C. before use.

Four samples (two lanes of undiluted sample and two lanes of 2× dilution) were loaded onto an 10% SDS-PAGE gel. Electrophoresis was performed for 2 hours at 200V. The gel was stained by Coomassie Brilliant Blue for 1 h at room temperature and destained overnight. The destained Coomassie Blue stained gel is shown in FIG. 11B.

Proteins were transferred to the membrane at 400 mAmp for 2 hours. The membrane was blocked in 5% BSA overnight. Primary polyclonal rabbit antibodies (1:500 dilution) were added to the membrane for 1.5 hours. Secondary HRP-conjugated goat anti-rabbit antibody (1:10,000 dilution) was added to the membrane for 1 hour and the membrane was stained in 1% diaminobenzidine. FIG. 11A shows the specific immune detection. Lanes 3 and 4 are replicates of lanes 1 and 2 but comprising ½ the protein load.

A further aspect includes an isolated antibody that detects Brachyspira sp. Sask30446.

The term “antibody” as used herein is intended to include monoclonal antibodies, polyclonal antibodies, and chimeric antibodies. The antibody may be from recombinant sources and/or produced in transgenic animals.

The term “antibody binding fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies may be monospecific, bispecific, trispecific or of greater multispecificity. Multispecific antibodies may immunospecifically bind to different epitopes of a Brachyspira sp. Sask30446 polypeptide and/or or a solid support material. Antibodies may be from any animal origin including birds and mammals (e.g., human, murine, donkey, sheep, rabbit, goat, guinea pig, camel, horse, or chicken).

Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies.

In aspects, the antibody is a purified or isolated antibody. By “purified” or “isolated” is meant that a given antibody or fragment thereof, whether one that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

Compositions comprising sufficient numbers of live Brachyspira sp. Sask30446 can induce mucohemorrhagic diarrhea in swine similar to a causative agent of swine dysentery as described further below.

Swine dysentery is a diarrheal illness caused by Brachyspira hyodysenteriae and characterized by profuse muco-hemorrhagic colitis. Grow-finish pigs have been recently observed with clinical signs indistinguishable from swine dysentery where no B. hyodysenteriae could be detected in spite of large numbers of spirochetes seen on fecal smears. Based on NADH oxidase gene sequences, these spirochetes, referred to as Brachyspira sp. Sask30446 (or optionally B. sp. ‘campestris’), are phylogenetically distinct from recognized Brachyspira spp. An infection trial was conducted using colonic mucosal scrapings from field cases to demonstrate transmissibility, and to investigate the association between spirochetal shedding and diarrhea. Twelve inoculated and 6 control pigs were housed in separate rooms and fed a commercially prepared, non-medicated, pelleted diet.

Following three consecutive days of inoculation, nine of 12 inoculated animals developed muco-hemorrhagic diarrhea. All controls remained healthy. The number of spirochetes seen on fecal smears increased with the onset of diarrhea and significantly more spirochetes were seen on colonic mucosal smears from inoculated than control animals. The concentration of B. sp. Sask30446 DNA (genome copiesg) was also significantly higher in pigs that developed muco-hemorrhagic diarrhea than in control or inoculated animals without diarrhea. B. hyodysenteriae and B. pilosicoli were not isolated from any animal. The trial demonstrates that a significant relationship exists between B. sp. Sask30446 and diarrhea.

Grow-finish pigs with muco-hemorrhagic diarrhea consistent with swine dysentery were observed in a commercial barn in Saskatchewan, Canada. Tissues, carcasses and rectal swabs collected from a number of affected pigs over several months were analysed. Fibrinous muco-hemorrhagic colitis and typhlitis with superficial necrosis were seen on necropsy. Histologically, sub-acute to chronic muco-purulent to fibrino-suppurative colitis was seen. All samples were negative for B. hyodysenteriae, B. pilosicoli, and Lawsonia intracellularis by PCR, and Salmonella spp. by culture. Although large numbers of spirochetes were visible on Gram stained fecal smears, attempts to culture Brachyspira were unsuccessful. A diagnostic investigation was initiated to determine the cause of colitis using archived and new samples.

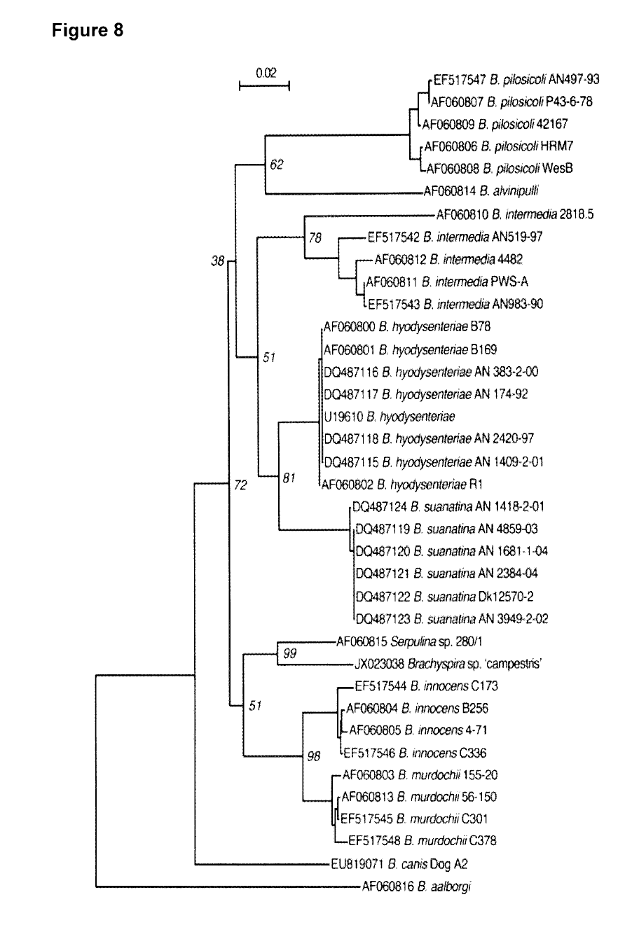

To identify the spirochetes seen in the feces, samples were tested by PCR using previously published primers for the NADH oxidase (nox) gene, a target universal to Brachyspira species and widely used for species identification in this genus.⁸ A phylogenetically distinct nox sequence, that is as different from recognized Brachyspira species as recognized species are from each other was detected (GenBank accession number JX023038) (FIG. 8).^{3,4} It is believed the organism is a new species, and is referred to as Brachyspira sp. Sask30446 (or Brachyspira sp. ‘campestris’). Multiple, epidemiologically unrelated cases have since been identified.

At the time the study was initiated it was not possible to culture B. sp. Sask30446, therefore homogenized colonic mucosal scrapings from pigs with B. sp. Sask30446 associated muco-hemorrhagic diarrhea were used to infect healthy pigs. In addition to reproducing disease, spirochete shedding in relation to clinical disease was evaluated by microscopic analysis of fecal smears.

B. sp. Sask30446 infected material used in this study was obtained from clinically affected 13-week-old pigs from a PRRS negative nursery-finish barn. Following necropsy, the colonic and caecal mucosa (the inoculum) were removed from the underlying submucosa and muscularis by scraping with the edge of a glass microscope slide, then frozen at −80° C. within four hours of collection. To confirm the absence of pathogens other than B. sp. Sask30446, sections of small and large intestine were processed for histopathology, bacterial culture, and PCR.

For detection of Brachyspira spp. in tissue samples, a SYBR green quantitative PCR was performed using primers targeting either the nox gene (Brachyspira sp. Sask30446) (JH0224 5′TCG CTA AAT TAT TCC AAC MG GA-3′ (SEQ ID NO: 3), JH0225 5′AAA CGC ATT TCT ATT CCA GCA-3′) (SEQ ID NO: 4) or the cpn60 gene (B. hyodysenteriae, B. pilosicoli) (JH0073 5′ AGT GM ATA GTT GCT CAT ATC AAA T-3′ (SEQ ID NO: 38), JH0074 5′ GCA TCA CTG ATT AAA GM CCA AT-3′ (SEQ ID NO: 39)) and (JH0077 5′ ACA ATG ATA MG AGA TAG GTG CTT-3′ (SEQ ID NO: 40), JH0078 5′ CTA ATG AAA GGC TAG TTT CTA ATG AT-3′ (SEQ ID NO: 41). Total DNA was extracted from tissue samples using either the QIAmp DNA stool mini kit or DNEasy blood and tissue kit (Qiagen Inc, Toronto Ontario) and 2 μl of extract used as template in PCR reactions. All PCR reactions were conducted on a Bio-Rad MyiQ thermocycler with iQ SYBR green supermix (Bio Rad Laboratories, Missisauga, Ontario) according to the manufacturer's instructions. Quantification was accomplished by use of a serially diluted standard curve of plasmids containing target sequences. The lower limit of detection for all assays was ˜10² copies per reaction (˜10⁴ copies per gram of starting material). All reactions were run in duplicate and each run included both extraction negatives and no template controls. For samples that resulted in a Cₜ value beyond the lowest standard, but with dissociation curves consistent with the expected product, a result of detected but not quantifiable (DNQ) was reported.

For this study, 18 five-week old, male weanling piglets were purchased from a high health commercial farrow to finish barn in Saskatchewan, Canada with no history of swine dysentery or previous laboratory diagnosis of Brachyspira spp. Farm selection was based on screening four and seven week old pigs from three farms for B. hyodysenteriae, B. pilosicoli and B. sp. Sask30446. The chosen facility had the lowest rate of B. sp. Sask30446 detection, 1 of 20 detectable but not quantifiable (DNQ), and 19 of 20 negative, and no B. hyodysenteriae or B. pilosicoli detected. The purchased pigs were conveniently selected, of average body weight compared to their cohorts, and appeared healthy.

On arrival animals were randomly assigned to control (CTRL, n=6) and inoculated (INOC, n=12) groups and held for a 10 day acclimation period prior to inoculation. They were fed a commercially prepared, non-medicated, pelleted starter diet ad libitum and housed in separate rooms in 4′×6′ pens containing 3 pigs. During acclimation all pigs were tested for B. hyodysenteriae, B. pilosicoli and B. sp. Sask30446 by quantitative PCR at five, seven and ten days prior to inoculation. Three INOC pigs were positive (DNQ) for B. sp. Sask30446 at single time points; one at 10 days prior to inoculation and two at five days prior to inoculation. Neither B. hyodysenteriae nor B. pilosicoli were detected.

An inoculum was prepared fresh daily by mixing approximately one part mucosal scraping and three parts phosphate buffered saline (0.1 M pH 7.0) (PBS). The final inoculum had an average concentration of Brachyspira sp. Sask30446 of 1.95×10⁶ genomes/ml by PCR yielding an average dose of 1.95×10⁸ genome equivalents per 100 ml. This dose was intermediate to recent trials with B. murdochii where 10⁶ colony/forming units were used and a ‘B. suanatina’ trial where 30 ml of a 10⁸ to 10⁹ inoculum was used.^{5,7}

Pigs were inoculated on 3 consecutive days (study days 0, 1 and 2) as done in previous Brachyspira infection trials.^{5,7} To decrease gastric transit time, feed was removed the evening prior to inoculation about 15-17 hours before inoculation. Briefly, pigs were sedated with azaperone (Vetoquinol Canada, Lavaltrie Quebec)(8 mg/kg IM), an 18 FR stomach tube (Benlan Inc, Oakville, Ontario) was passed, and 100 ml of tissue homogenate (INOC) or sterile PBS (CTRL) was administered followed by 50 ml of sterile PBS to flush residual inoculum into the stomach.

The pigs were observed twice daily; responsiveness, skin colour, appetite and body condition, feces score, respiratory effort and temperature were scored and recorded. The fecal score reflected the consistency and presence of blood and mucus. Gram stained fecal smears were made from rectal swabs collected from each pig daily. Slides were semi-quantitatively scored (0: no spirochetes seen, 1+: ≦1 spirochete/1000× field, 2+: 2-10 spirochetes/1000× field, 3+: 11-49 spirochetes/1000× field, 4+: >50 spirochetes/1000× field) by a single investigator who was blinded to the identity of the slide.

Pigs were euthanized by cranial captive bolt and exsanguination after the intensity of muco-hemorrhagic diarrhea peaked (INOC) or at the end of the study (CTRL). A complete necropsy was performed; special attention was paid to the stomach, mesenteric lymph nodes, duodenum, jejunum, ileum, spiral colon, caecum and rectum. Samples for histological examination, Salmonella culture, Lawsonia intracellularis PCR and PCV2 immunohistochemistry were analyzed.

Statistical analysis was performed using SPSS version 18.0 (SPSS Inc., Chicago). The presence of muco-hemorrhagic diarrhea in INOC vs. CTRL groups was compared using Fisher's exact test. The presence or absence of histologic and gross lesions in pigs with and without muco-hemorrhagic diarrhea and CTRL pigs was compared using Fisher's exact test. Terminal spirochete slide scores and B. sp. Sask30446 concentration (either negative, DNQ or quantifiable) in pigs with muco-hemorrhagic diarrhea, inoculated pigs without muco-hemorrhagic diarrhea and CTRL were compared using the Kruskal-Wallis AOV followed by post-hoc Mann-Whitney test if significant. Two-tailed P-values 50.05 were considered significant.

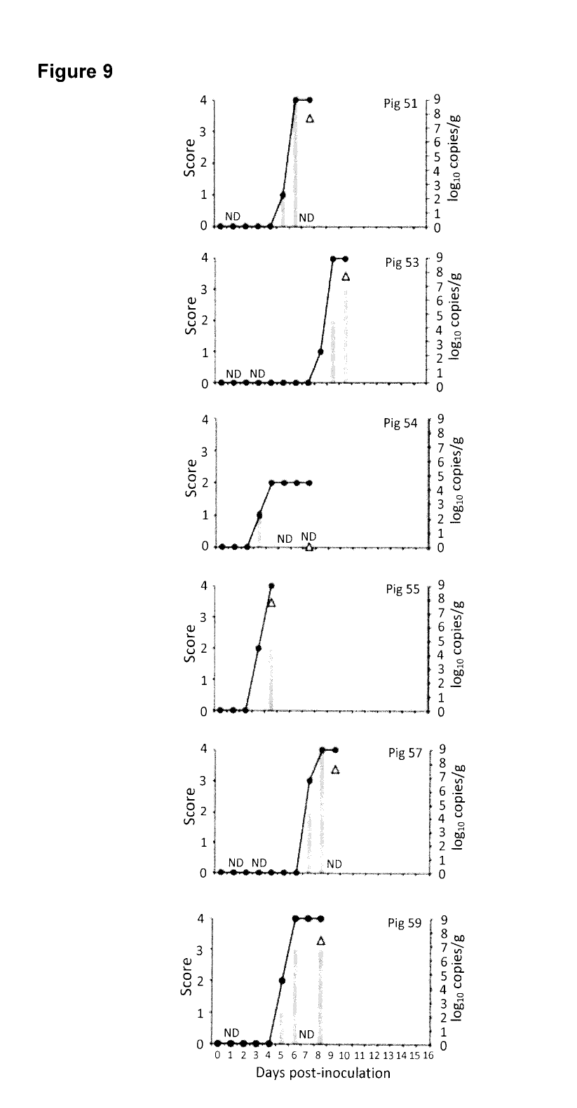

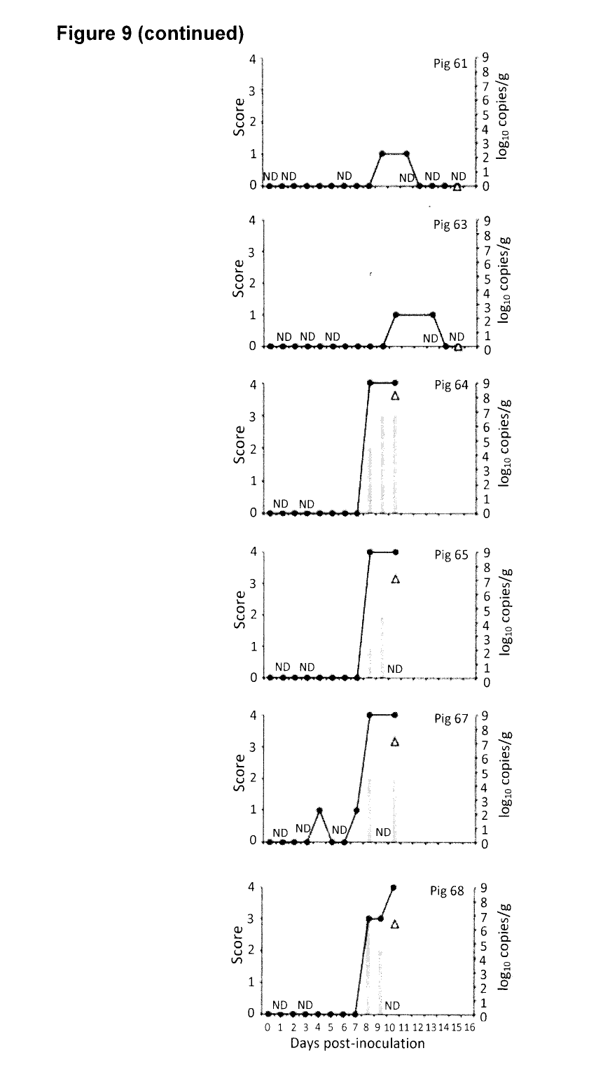

The study continued for 16 days following the first inoculation. All CTRL animals remained healthy throughout the study. Spirochetes were never seen in the feces nor detected by quantitative PCR. All samples collected at termination were negative for all pathogens tested. Of the 12 INOC pigs, nine developed muco-hemorrhagic diarrhea between days 4 and 10 post inoculation (PI) (FIG. 9). Of the remaining three INOC pigs, two (#61 and #63) developed soft feces on days nine and ten, that resolved on days 12 and 14 PI respectively. Pig #54 developed watery diarrhea on day four PI. Neither blood nor mucous were observed from any of these animals. Muco-hemorrhagic diarrhea was significantly more common in INOC (9 of 12) than CTRL (0 of 6) (P<0.001). In pigs that developed muco-hemorrhagic diarrhea, the number of spirochetes seen on Gram stained slides increased on the first or second day of elevated fecal scores (diarrhea) (FIG. 9). In one INOC pig (#67), transient mild diarrhea was observed four days before the first spirochetes were seen, and three days prior to the onset of persistent diarrhea (FIG. 9). Of the three INOC pigs that did not develop muco-hemorrhagic diarrhea, spirochetes were not observed from two (#61 and #63), transient low level spirochete shedding was observed on a single day for the other (#54) (FIG. 9). Significantly higher terminal spirochete slide scores were seen in pigs with muco-hemorrhagic diarrhea than either INOC pigs without muco-hemorrhagic diarrhea (P=0.011), or CTRL (P<0.001).

Lawsonia intracellularis, B. hyodysenteriae, B. pilosicoli, Salmonella spp., TGE and PRRS were not detected in terminal samples from any pig. A single INOC pig (#57) was weakly positive for PCV2, all other animals were negative. The concentration of B. sp. Sask30446 DNA detected by quantitative PCR was consistent with clinical observations and spirochete slide score (FIG. 9). Terminal B. sp. Sask30446 concentrations were significantly higher in pigs with muco-hemorrhagic diarrhea than either INOC without muco-hemorrhagic diarrhea (P=0.009), or CTRL (P<0.001).

Gross pathological findings were consistent with clinical signs; with more severe lesions seen in pigs with muco-hemorrhagic diarrhea than in INOC pigs without muco-hemorrhagic diarrhea. In affected pigs, enlargement of the mesenteric lymph nodes, fibrinous typhlitis, meso-colonic edema and/or congestion and abundant muco-hemorrhagic caecal colonic and rectal contents were seen. No abnormal findings were observed in CTRL pigs.

For histopathological examination, changes in the large intestines of all pigs were scored for inflammation and necrosis by one pathologist who was blinded to the status of the pigs.

Lesions were detected in the large intestines of all nine pigs with muco-hemorrhagic diarrhea (Table 2). The lesions involved necrosis of the superficial mucosa which was covered by a bacteria-rich, mucoid exudate that extended into the superficial crypts. When examined using Warthin-Faulkner silver stains, numerous long thin spirochetes were seen in the crypts and among the mixed bacteria on the surface. There was mild but variable congestion and hemorrhage in the lamina propria and a mild neutrophil infiltration in the lamina propria and lumen in some cases. Lesions were most consistent in the colon but were also detected in the caecum and rectum (Table 2). Lesions among the three inoculated pigs that did not develop muco-hemorrhagic diarrhea were mild and similar to the normal background lesions in the control pigs.

This study demonstrates that the emerging swine-dysentery-like disease is transmitted by the fecal-oral route. Interestingly, the incubation period observed here (4-10 days) is similar to a 1921 infection which utilized intestinal contents, where a 5-11 day incubation period was seen.¹⁰ The significant association of disease with a novel Brachyspira sequence and spirochetes in the feces strongly suggests the emergence of an unidentified pathogen.

Specific etiological diagnosis of brachyspirosis is challenging. The lack of pathognomonic clinical or pathological findings associated with B. sp. Sask30446 necessitates reliance on laboratory tests to differentiate it from B. hyodysenteriae or B. murdochii. Negative results should be interpreted with caution as current methods do not target B. sp. Sask30446. Similar to a previous trial with B. hyodysenteriae, the presence of spirochetes in the feces was predictive of diarrhea suggesting that evaluation of fecal smears is an invaluable diagnostic test.⁶

At present, it is not known how widespread B. sp. Sask30446 infection or disease is. Similarities in its clinical presentation with classical swine dysentery and the inability of published diagnostic tests to detect and differentiate it, suggest that its importance may be underappreciated. Presently, cases have been described in Alberta, Saskatchewan, and the Midwestern United States.^{4, 9, 11}

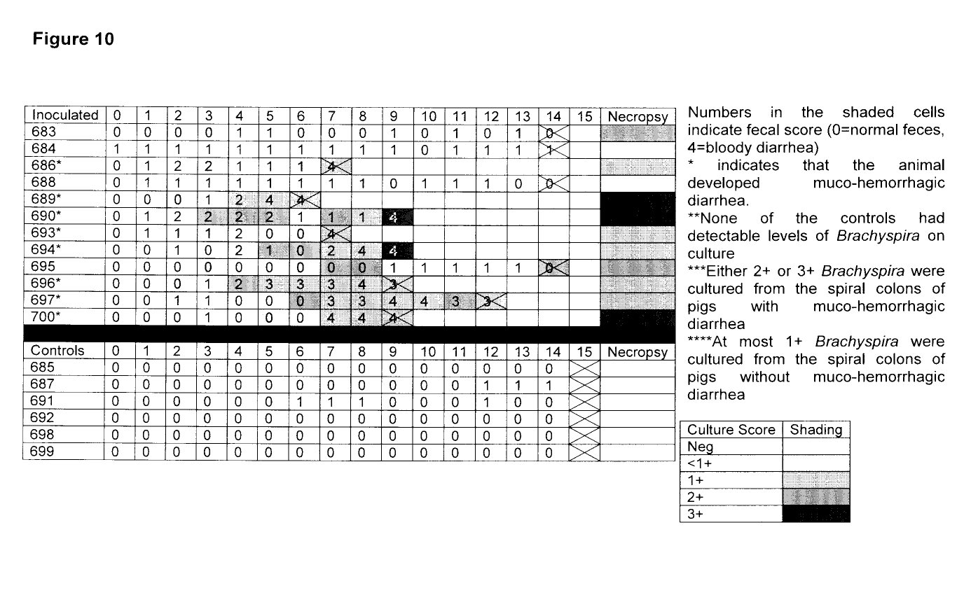

A second inoculation experiment was conducted and the results are presented in Table 3 and summarized in FIG. 10.

The second inoculation trial used liquid culture grown Brachyspira sp. Sask30446 propagated and isolated according to a method described herein. Inoculations were conducted similar to as described above except the source of Brachyspira sp. Sask30446 was isolated from infected swine and liquid culture propagated. The heat-map in FIG. 10 shows daily fecal scores and culture results for all experimental and control pigs. qPCR data was also obtained for all samples and supports these observations and agrees well with clinical signs.

Additional characteristics of Brachyspira sp. Sask30446 are provided in Table 4 and Table 5. These characteristics are useful for distinguishing B. sp. Sask30446, for example from B. hyodysenteriae or B. pilosicoli.

TABLE 2
Pathological lesions and observation of spirochetes on histological examination

| | | | | Gross Lesions | | | | |
|---|---|---|---|---|---|---|---|---|
| | | | | Colon | | | | |
| | | | | Mucoid | | Caecum | Spirochetes on | |
| | | | | and/or | | Muco- | Silver Stain | |
| | Histologic Lesions | | | hemorrhagic | Edema and | fibrinous | Absent- | |
| | Colon | Caecum | Rectum | colitis | congestion | typhlitis | Rare | Abundant |
| Control | 0/6^{†} | 0/6^{†} | 0/6^{†} | 0/6^{†} | 0/6^{†} | 0/6^{†} | 2/6 | 0/6 |
| Inoculated | 9/12 | 5/12 | 6/12 | 7/12 | 9/12 | 7/12 | 3/12 | 9/12 |
| Diarrheic | 9/9*^{†} | 5/9^{†} | 5/9^{†} | 7/9*^{†} | 9/9*^{†} | 7/9*^{†} | 0/9 | 9/9 |
| No diarrhea | 0/3* | 0/3 | 1/3 | 0/3* | 0/3* | 0/3* | 3/3 | 0/3 |

Comparison of histological and gross lesions and abundance of spirochetes on Warthin-Faulkner stained colonic sections between CTRL (n = 6), diarrheic (n = 9) and INOC without diarrhea (n = 3).
Significant differences (P ≦ 0.05) are denoted by superscript symbols.

TABLE 3
Raw data

| 683 | 0 | 1 | 2 | 3 | 4 | 5 | 6 | 7 | 8 | 9 | 10 | 11 | 12 | 13 | 14 | day post-inoculation |
|---|---|---|---|---|---|---|---|---|---|---|---|---|---|---|---|---|
| Fecal | 0 | 0 | 0 | 0 | 1 | 1 | 0 | 0 | 0 | 1 | 0 | 1 | 0 | 1 | 0 | |
| Culture | 0 | 0 | 0 | 0 | 0 | 0 | 0 | 0 | 0 | 0 | 0 | 0 | 0 | 1 | 0 | |
| Slide | | | | | | | | | | | | | | | | |
| qPCR | Neg | Neg | Neg | Neg | 4 | Neg | Neg | Neg | Neg | Neg | 4 | Neg | Neg | Neg | Neg | |
| 684 | 0 | 1 | 2 | 3 | 4 | 5 | 6 | 7 | 8 | 9 | 10 | 11 | 12 | 13 | 14 | day post-inoculation |
| Fecal | 1 | 1 | 1 | 1 | 1 | 1 | 1 | 1 | 1 | 1 | 0 | 1 | 1 | 1 | 1 | |
| Culture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | 4 | Neg | Neg | Neg | Neg | Neg | Neg | Neg | Neg | Neg | 5 | Neg | Neg | Neg | Neg | |
| 686 | 0 | 1 | 2 | 3 | 4 | 5 | 6 | 7 | 8 | 9 | 10 | 11 | 12 | 13 | 14 | day post-inoculation |
| Fecal | 0 | 1 | 2 | 2 | 1 | 1 | 1 | 4 | ND | ND | ND | ND | ND | ND | ND | |
| Culture | 0 | 0 | 0 | 1 | 0 | 0 | 1 | 3 | ND | ND | ND | ND | ND | ND | ND | |
| Slide | | | | | | | | | ND | ND | ND | ND | ND | ND | ND | |
| qPCR | Neg | Neg | DNQ | Neg | Neg | 6 | 5 | 6 | ND | ND | ND | ND | ND | ND | ND | |
| 688 | 0 | 1 | 2 | 3 | 4 | 5 | 6 | 7 | 8 | 9 | 10 | 11 | 12 | 13 | 14 | day post-inoculation |
| Fecal | 0 | 1 | 1 | 1 | 1 | 1 | 1 | 1 | 1 | 0 | 1 | 1 | 1 | 0 | 0 | |
| Culture | 0 | 0 | 0 | 0 | 1 | 0 | 0 | 0 | 0 | 0 | 0 | 0 | 0 | 0 | 0 | |
| Slide | | | | | | | | | | | | | | | | |
| qPCR | Neg | Neg | Neg | Neg | Neg | Neg | Neg | Neg | Neg | Neg | Neg | Neg | Neg | Neg | Neg | |
| 689 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 1 | 2 | 4 | 4 | ND | ND | ND | ND | ND | ND | ND | ND | |
| Culture | 0 | 0 | 1 | 0 | 3 | 3 | 3 | ND | ND | ND | ND | ND | ND | ND | ND | |
| Slide | | | | | | | | ND | ND | ND | ND | ND | ND | ND | ND | |
| qPCR | Neg | Neg | Neg | DNQ | 5 | 7 | 8 | ND | ND | ND | ND | ND | ND | ND | ND | |
| 690 | 0 | 1 | 2 | 3 | 4 | 5 | 6 | 7 | 8 | 9 | 10 | 11 | 12 | 13 | 14 | day post-inoculation |
| Fecal | 0 | 1 | 2 | 2 | 2 | 2 | 1 | 1 | 1 | 4 | ND | ND | ND | ND | ND | |
| Culture | 0 | 0 | 0 | 2 | 2 | 2 | 0 | 2 | 3 | 4 | ND | ND | ND | ND | ND | |
| Slide | | | | | | | | | | | ND | ND | ND | ND | ND | |
| qPCR | DNQ | Neg | Neg | Neg | Neg | Neg | 5 | 4 | 5 | 6 | ND | ND | ND | ND | ND | |
| 693 | 0 | 1 | 2 | 3 | 4 | 5 | 6 | 7 | 8 | 9 | 10 | 11 | 12 | 13 | 14 | day post-inoculation |
| Fecal | 0 | 1 | 1 | 1 | 2 | 0 | 0 | 4 | ND | ND | ND | ND | ND | ND | ND | |
| Culture | 0 | 0 | 0 | 1 | 0 | 0 | 1 | 3 | ND | ND | ND | ND | ND | ND | ND | |
| Slide | | | | | | | | | ND | ND | ND | ND | ND | ND | ND | |
| qPCR | Neg | Neg | Neg | Neg | 4 | Neg | Neg | 6 | ND | ND | ND | ND | ND | ND | ND | |
| 694 | 0 | 1 | 2 | 3 | 4 | 5 | 6 | 7 | 8 | 9 | 10 | 11 | 12 | 13 | 14 | day post-inoculation |
| Fecal | 0 | 0 | 1 | 0 | 2 | 1 | 0 | 2 | 4 | 4 | ND | ND | ND | ND | ND | |
| Culture | 0 | 0 | 0 | 0 | 1 | 2 | 2 | 3 | 3 | 4 | ND | ND | ND | ND | ND | |
| Slide | | | | | | | | | | | ND | ND | ND | ND | ND | |
| qPCR | Neg | Neg | Neg | Neg | Neg | Neg | 6 | 7 | 7 | 7 | ND | ND | ND | ND | ND | |
| 695 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 0 | 0 | 0 | 1 | 1 | 1 | 1 | 1 | 0 | |
| Culture | 0 | 0 | 0 | 1 | 0 | 0 | 1 | 2 | 2 | 0 | 0 | 0 | 0 | 0 | 2 | |
| Slide | | | | | | | | | | | | | | | | |
| qPCR | DNQ | Neg | Neg | Neg | 4 | Neg | Neg | 5 | Neg | Neg | 4 | Neg | 4 | Neg | Neg | |
| 696 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 1 | 2 | 3 | 3 | 3 | 4 | 3 | ND | ND | ND | ND | ND | |
| Culture | 0 | 0 | 1 | 0 | 2 | 3 | 3 | 3 | 3 | 3 | ND | ND | ND | ND | ND | |
| Slide | | | | | | | | | | | ND | ND | ND | ND | ND | |
| qPCR | DNQ | Neg | Neg | Neg | 5 | Neg | 7 | 6 | 7 | 6 | ND | ND | ND | ND | ND | |
| 697 | 0 | 1 | 2 | 3 | 4 | 5 | 6 | 7 | 8 | 9 | 10 | 11 | 12 | 13 | 14 | day post-inoculation |
| Fecal | 0 | 0 | 1 | 1 | 0 | 0 | 0 | 3 | 3 | 4 | 4 | 3 | 3 | ND | ND | |
| Culture | 0 | 0 | 0 | 0 | 0 | 0 | 2 | 3 | 3 | 3 | 3 | 2 | 3 | ND | ND | |
| Slide | | | | | | | | | | | | | | ND | ND | |
| qPCR | Neg | Neg | Neg | Neg | Neg | Neg | 4 | 6 | 7 | 7 | No | 6 | 5 | ND | ND | |
| | | | | | | | | | | | sample | | | | | |
| 700 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 1 | 0 | 0 | 0 | 4 | 4 | 4 | ND | ND | ND | ND | ND | |
| Culture | 0 | 0 | 0 | 0 | 0 | 1 | 0 | 3 | 3 | 3 | ND | ND | ND | ND | ND | |
| Slide | | | | | | | | | | | ND | ND | ND | ND | ND | |
| qPCR | Neg | Neg | Neg | Neg | Neg | Neg | 5 | 6 | 6 | 6 | ND | ND | ND | ND | ND | |
| 685 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 0 | 0 | 0 | 0 | 0 | 0 | 0 | 0 | 0 | |
| Culture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | DNQ | Neg | Neg | Neg | Neg | Neg | Neg | Neg | Neg | Neg | Neg | Neg | Neg | Neg | Neg | |
| 687 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 0 | 0 | 0 | 0 | 0 | 0 | 1 | 1 | 1 | |
| Culture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | Neg | Neg | Neg | Neg | Neg | Neg | Neg | Neg | Neg | Neg | Neg | Neg | Neg | Neg | Neg | |
| 691 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 1 | 1 | 1 | 0 | 0 | 0 | 1 | | | |
| Culture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | DNQ | Neg | Neg | Neg | Neg | Neg | Neg | Neg | Neg | Neg | Neg | Neg | Neg | Neg | Neg | |
| 692 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 0 | 0 | 0 | 0 | 0 | 0 | 0 | 0 | 0 | |
| Cutture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | Neg | Neg | Neg | Neg | Neg | Neg | Neg | Neg | Neg | Neg | Neg | Neg | Neg | Neg | Neg | |
| 698 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 0 | 0 | 0 | 0 | 0 | 0 | 0 | 0 | 0 | |
| Culture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | Neg | Neg | Neg | Neg | Neg | Neg | Neg | Neg | Neg | Neg | Neg | Neg | Neg | Neg | Neg | |
| 699 | 0 | 1 | 2 | 3 | 4 | 5 | 6 | 7 | 8 | 9 | 10 | 11 | 12 | 13 | 14 | day post-inoculation |
| Fecal | 0 | 0 | 0 | 0 | 0 | 0 | 0 | 0 | 0 | 0 | 0 | 0 | 0 | 0 | 0 | |
| Culture | 0 | 0 | 0 | 0 | 0 | 0 | 0 | 0 | 0 | 0 | 0 | 0 | 0 | 0 | 0 | |
| Slide | | | | | | | | | | | | | | | | |
| qPCR | Neg | Neg | Neg | Neg | Neg | Neg | Neg | Neg | Neg | Neg | Neg | Neg | Neg | Neg | Neg | |

TABLE 4
Characteristics of Brachyspira sp. Sask30446 (aka B. campestris)

| Characteristic | B. sp Sask30446. |
|---|---|
| Average cell length^{b} | 8.53 μm ± 2.19 μm |
| Average cell diameter^{b} | 0.46 μm ± 0.05 μm |
| No. flagella | 12-15 |
| β-hemolysis | Strong |
| Indole production | − |
| Hippurate hydrolysis | − |
| α-galactosidase | − |
| α-glucosidase | − |
| β-glucosidase | + |
| Pathogenicity for swine | Dysentery-like |
| | Muco-hemorrhagic diarrhea |

^{b}cell measurements are the average of 20 cells +/− 1 standard deviation the number of flagella refers to the number of flagella inserted at each end of the cell. These measurements were made on electron micrographs
The alpha and beta glucosidase and alpha galactosidase activity were determined using API zym (a commercially prepared kit from BioMerieux)

TABLE 5
Comparison of acid production by various Brachyspira species

| | Production of acid^{a} | | |
|---|---|---|---|
| | | B. hyodysenteriae | B. pilosicoli |
| Carbohydrate | B. sp. Sask30446 | ATCC 27164 | ATCC 51139 |
| D-cellobiose | − | + | + |
| D-fructose | − | − | + |
| D-galactose | − | − | + |
| D-glucose | − | + | + |
| D-maltose | − | − | − |
| D-mannose | − | + | + |
| D-ribose | − | − | + |
| D-trehalose | − | − | − |
| L-fructose | − | − | + |
| N-acetyl-D- | − | − | + |
| glucosamine | | | |

^{a}Acid production was defined by a ≧0.5 pH unit lower pH in broths containing organism and the carbohydrate compared to broths without carbohydrate.

Among recognized species of Brachyspira, B. sp. Sask30446 can be differentiated for example based on strong beta-hemolysis, lack of indole production, and failure to hydrolyze hippurate. With completely unknown samples, sequence information can be used to conclusively identify Brachyspira sp. Sask30446.

Accordingly, in an embodiment the isolated Brachyspira sp. Sask30446 is strongly hemolytic, exhibits ring phenomenon (i.e. is ring phenomenon positive), lacks indole production, and fails to hydrolyze hippurate. In an embodiment, such phenotypic characteristics including for example lack of acid production from the substrates listed in Table 5 is used to discriminate Brachyspira sp. Sask30446 from Brachyspira hyodysenteriae and/or Brachyspira pilosicoli

In an embodiment, the isolated Brachyspira sp. Sask30446 is strongly hemolytic, exhibits ring phenomenon and exhibits each of the characteristics described in Table 4 and/or 5.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### CITATIONS FOR REFERENCES REFERRED TO IN THE SPECIFICATION

1 Christopher-Hennings J, Nelson E A, Nelson J K, et al.: 1995, Detection of porcine reproductive and respiratory syndrome virus in boar semen by PCR. J Clin Microbiol 33:1730-1734.
2 Hampson D J, Fellstrom C, Thomson J R: 2006, Swine Dysentery. In: Diseases of Swine, eds. Straw B E, Zimmerman J J, D'Allaire STaylor DJ, 9th ed., pp. 785-805. Blackwell Publishing, Ames, 10.
3 Harding J C S, Chirino-Trejo M, Fernando C, et al.: 2010, Detection of a novel Brachyspira species associated with haemorrhagic and necrotizing colitis. In: International Pig Veterinary Society Congress, p. 740. Vancouver, Canada.
4 Harding J C S, Hill J E, Chirino-Trejo M, et al.: 2011, Detection of a novel Brachyspira species associated with haemorrhagic and necrotizing colitis. In: Carlos Pijoan Symposium on Swine Dysentery, pp. 27-32. St. Paul, Minn.
5 Jensen T K, Christensen A S, Boye M: 2010, Brachyspira murdochii colitis in pigs. Vet Pathol 47:334-338.
6 Olson L D, Fales W H: 1983, Comparison of stained smears and culturing for identification of Treponemahyodysenteriae. J Clin Microbiol 18:950-955.
7 Rasback T, Jansson D S, Johansson K E, Fellstrom C: 2007, A novel enteropathogenic, strongly haemolytic spirochaete isolated from pig and mallard, provisionally designated ‘Brachyspira suanatina’ sp. nov. Environ Microbiol 9:983-991.
8 Rohde J, Rothkamp A, Gerlach G F: 2002, Differentiation of porcine Brachyspira species by a novel nox PCR-based restriction fragment length polymorphism analysis. J Clin Microbiol 40:2598-2600.
9 Schwartz K: 2011, Brachyspira: What's Happening in Iowa and Why. In: Western Canadian Association of Swine Veterinarians Conference, pp. 25-33.
10 Whiting R A, Doyle L P, Spray R S: 1921, Swine Dysentery. Bulletin:3-15.
11. Burrough E, Strait E, Kinyon J, Bower L, Madson D, Schwartz K, Frana T, Songer J G. Comparison of atypical Brachyspira spp. Clinical isolates and classic strains in a mouse model of swine dysentery. Vet. Microbiol. (2012) http://dxdoi.org/10.1016/j.vetmic.2012.06.008.

1. A method of culturing a Brachyspira sp. Sask30446 organism comprising at least 96% sequence identity to SEQ ID NOs: 7, 8 and 9; at least 92.3% sequence identity to SEQ ID NOs: 11 and 12; at least 97.5% sequence identity to SEQ ID NO: 25; at least 98.5% sequence identity to SEQ ID NO:26; at least 93.5% sequence identity to SEQ ID NO:27 and 31; at least 95.5% sequence identity to SEQ ID NO:28, 29 and 34; at least 99.5% sequence identity to SEQ ID NO: 30, 32 and 37 and at least 92.5% identity to SEQ ID NO:33, the method comprising inoculating an agar media and/or a liquid media with a sample comprising the Brachyspira sp. Sask30446 organism and incubating the agar media and/or the liquid media at a temperature between 25-44° C. under anaerobic conditions, wherein the liquid media comprises BHI, 1-20% blood product and 0.5% to 10% glucose, optionally wherein the agar media corn irises BAM, BJ, CVS or Blood agar media or a modified media thereof.

2-4. (canceled)

5. The method of claim 1, wherein the method further comprises subculturing or passaging the Brachyspira sp. Sask30446 one or more passages, wherein the subculturing comprises inoculating a further liquid media or a further agar media with portion of the agar media comprising a hemolytic zone comprising Brachyspira sp. Sask30446 organism.

6. (canceled)

7. The method of claim 1, the method comprising:
a) inoculating the agar media optionally BJ agar media or CVS agar media or a modified media thereof, with a sample comprising the Brachyspira sp. Sask30446 organism;
b) incubating the agar media, optionally BJ agar media or the CVS agar media or a modified media thereof at a temperature between 25-44° C., optionally between about 38-44° C. or between 40-43° C., preferably about 42° C. under anaerobic conditions for about 24 to 72 hrs, optionally about 40 to 56 his, preferably about 48 hrs until one or more zones of hemolysis are visible; and
c) subculturing the one or more zones of hemolysis.

8. (canceled)

9. The method of claim 1, wherein the sample is gastrointestinal tissue, gastrointestinal content and/or fecal material for example collected by fecal swab.

10. (canceled)

11. The method claim 7, wherein the subculturing comprises culturing the Brachyspira sp. Sask30446 in a liquid media comprising BHI and/or HI, 1-20% blood product and 0.5-10% glucose, preferably JBS media, wherein the liquid media is inoculated with the one or more zones of hemolysis comprising Brachyspira sp. Sask30446; and the inoculated liquid media is incubated under anaerobic conditions at a temperature between 25-44° C. to Provide an incubated culture.

12. (canceled)

13. The method of culturing a Brachyspira sp. Sask30446 organism in a sample obtained from a subject infected or suspected of being infected with the Brachyspira sp. Sask30446 organism, the method comprising:
a) obtaining the sample from the subject;
b) inoculating the agar media with the sample from the subject and incubating at a temperature of 25-44° C. under anaerobic conditions according to the method of claim 1 to obtain a colony and/or hemolytic zone;
c) optionally one of more subculturing steps; and
d) detecting the presence or absence of a polypeptide selected from a polypeptide comprising SEQ ID NO: 11, 12, 26, 28, 30, 32, and 34; a polypeptide having at least 92.3% sequence identity to SEQ ID NOs: 11 or 12; at least 98.5% sequence identity to SEQ ID NO: 26; at least 95.5% sequence identity to SEQ ID NO: 28; at least 99.5% sequence identity, to SEQ ID NO: 30; least 99.5% sequence identity to SEQ ID NO: 32 and/or at least 95.5% sequence identity to SEQ ID NO: 34; and/or a polynucleotide selected from a polynucleotide having at least 96% sequence identity to SEQ ID NOs: 7, 8 and/or 9; at least 97.5% sequence identity to SEQ ID NO: 25; at least 93.5% sequence identity to SEQ ID NO:27 and/or 31; at least 95.5% sequence identity to SEQ ID NO: 29; at least 99.5% sequence identity to SEQ ID NO: 37; at least 92.5% sequence identity to SEQ ID NO:33; and/or any combination thereof in the sample and/or in the hemolytic zone and/or colony which is indicative that the colony and/or hemolytic zone comprises Brachyspira sp. Sask30446.

14. The method of claim 13, wherein the method comprises a subculturing step, the subculturing step comprising inoculating media or an agar media with a colony and/or hemolytic zone and incubating the inoculated liquid media and/or solid media under anaerobic conditions at a temperature between 25-44° C. to provide an incubated culture, optionally wherein the liquid media comprises BHI and/or HI, 1-20% blood product and 0.5-10% glucose and/or the agar media is selected from BAM, BJ, CVS and Blood agar media and/or a modified media thereof.

15. (canceled)

16. (canceled)

17. A method of isolating a Brachyspira sp. Sask30446 organism comprising at least 96% sequence identity to SEQ ID NOs: 7, 8 and 9; at least 92.3% sequence identity to SEQ ID NOs: 11 and 12; at least 97.5% sequence identity to SEQ ID NO: 25; at least 98.5% sequence identity to SEQ ID NO:26; at least 93.5% sequence identity to SEQ ID NO:27 and 31; at least 95.5% sequence identity to SEQ ID NO:28, 29 and 34; at least 99.5% sequence identity to SEQ ID NO: 30, 32 and 37 and at least 92.5% sequence identity to SEQ ID NO:33; from a sample, the method comprising culturing a sample according to the method of claim 13 and extracting/isolating the Brachyspira sp. Sask30446 organism from the liquid media or agar media.

18. (canceled)

19. The method of claim 17, wherein the isolated Brachyspira sp. Sask30446 is frozen, dessicated or refrigerated.

20. (canceled)

21. An isolated Brachyspira sp. Sask30446 organism comprising one or more sequences of SEQ ID NOs: 7, 8 9, 11, 12, 25-34, 37; or at least 96% sequence identity to SEQ ID NOs: 7, 8 and 9, and at least 92.3% sequence identity to SEQ ID NOs: 11 and 12; at least 97.5% sequence identity to SEQ ID NO: 25; at least 98.5% sequence identity to SEQ ID NO:26; at least 93.5% sequence identity to SEQ ID NO:27 and 31; at least 95.5% sequence identity to SEQ ID NO:28, 29 and 34; at least 99.5% sequence identity to SEQ ID NO: 30, 32 and 37; and at least 92.5% sequence identity to SEQ ID NO:33; optionally wherein the Brachyspira sp. Sask30446 organism is packaged in a vial, such as a sterile vial and is optionally frozen, dessicated or refrigerated.

22. The isolated Brachyspira sp. Sask30446 organism of claim 21 comprising SEQ ID NOs: 7, 8 9, 11, 12, 25-34 and 37 or at least 99%, or 99.5% sequence identity to SEQ ID NOs: 7, 8, 9, 11, 12, 25-34 and 37.

23. (canceled)

24. The isolated Brachyspira sp. Sask30446 organism of claim 21 characterized by the bacteria strain deposited with the International Depository of Canada (1015 Arlington St., Suite H3390 Winnipeg, MB R3E 3R2) on Nov. 16, 2011 under Accession number 16111-01.

25. A composition comprising the isolated Brachyspira sp. Sask30446 of claim 21 and optionally a carrier or diluent.

26. (canceled)

27. The composition of claim 25, wherein the composition is an immunogenic composition and/or a pharmaceutical composition and the carrier or diluent is a pharmaceutically acceptable carrier or diluent.

28. The composition of claim 25, wherein the composition further comprises an isolated polypeptide of Brachyspira sp. Sask30446, selected from one or more polypeptides of SEQ ID NO:11, 12, 26, 28, 30, 3 and 34; a polypeptide having at least 92.3% sequence identity to any one of SEQ ID NOs: 11 and 12; at least 98.5% sequence identity to SEQ ID NO: 26; at least 95.5% sequence identity to SEQ ID NO: 28 at 99.5% sequence identity to SEQ ID NO: 30; at least 99.5% sequence identity to SEQ ID NO: 32; and/or at least 95.5% sequence identity to SEQ ID NO: 34; or a combination of two or more thereof and/or an adjuvant.

29.-31. (canceled)

32. A method for inducing an immune response in a subject against the isolated Brachyspira sp. Sask30446 organism of claim 21, the composition of claim 25 and/or a composition comprising said Brachyspira sp. Sask30446 organism and optionally further comprising an isolated polypeptide of any one of SEQ ID NO:11, 12, 26, 28, 30, 3 and 34; a polypeptide having at least 92.3% sequence identity to any one of SEQ ID NOs: 11 and 12; at least 98.5% sequence identity to SEQ ID NO: 26; at least 95.5% sequence identity to SEC ID NO: 28; at least 99.5% sequence identity to SEQ ID NO: 30; at least 99.5% sequence identity to SEQ ID NO: 32; and/or at least 95.5% sequence identity to SEQ ID NO: 34; or a combination of two or more thereof, comprising administering to the subject or a cell from the subject an effective amount of the isolated Brachyspira sp. Sask30446 organism or the isolated polypeptide.

33. The method of claim 32, wherein the subject is a swine, preferably a pig.

34. The method of claim 13, wherein the sample is obtained from a swine, preferably a pig with hemorrhagic colitis.

35. A process of obtaining a Brachyspira sp. Sask30446 organism comprising:
a) obtaining a sample from a subject infected or suspected of being infected with Brachyspira sp. Sask30446;
b) culturing the sample according to the method claim 1 to provide an incubated culture; and
c) optionally one or more subculturing steps;
d) determining a presence of a Brachyspira sp. Sask3.0446 polypeptide or polynucleotide in the sample and/or incubated culture and/or determining the phenotypic characteristic of the incubated culture; and
e) extracting/isolating Brachyspira sp. Sask30446 for example isolating hemolytic zone and/or colony from the solid media and/or isolating Brachyspira sp. Sask30446 from the incubated culture;
wherein detecting the presence of a Brachyspira sp. Sask30446 polypeptide or polynucleotide in the sample and/or incubated culture and/or detecting a hemolytic zone and/or colony and the presence of a hemolytic (e.g. on horse blood), tiny, clear, wet/glistening, “fried egg” shaped colony or zone of hemolysis on the solid agar media and/or optionally one or more phenotypic characteristics in Tables 4 and/or 5, indicates the extracted/isolated organism comprises Brachyspira sp. Sask30446.

36. A kit comprising an isolated Brachyspira sp. Sask30446 organism of claim 21 and one or more of a resuspension diluent, vial and/or instructions for use.

37. A media composition comprising Brain Heart Infusion broth, 1 to 20% blood product and 0.5% to 10% glucose, optionally comprising 5% ovine blood, 5% fetal calf serum and 1% glucose, and optionally comprising at least one antibiotic.

38. (canceled)

39. (canceled)

40. An isolated Brachyspira sp, comprising a DNA genome encoding SEQ ID NO: 11, 12, 26, 28 30, 32 and 34 and/or encoding polypeptides with at least 92.3% sequence identity with SEQ ID NO: 11 and 12; at least 98.5% sequence identity to SEQ ID NO:26; at least 95.5% sequence identity to SEQ ID NO:28 and 34; and at least 99.5% sequence identity to SEQ ID NO: 30 and 32.

41. The isolated Brachyspira sp. Sask30446 of claim 21 comprising genomic sequence with at least 95%, 96%, 97%, 98%, 99%, or at least 99.5% sequence identity to the bacteria strain deposited with the International Depository of Canada (IDAC) (1015 Arlington St., Suite H3390 Winnipeg, MB R3E 3R2) on Nov. 16, 2011 under Accession number 16111-01.

42. An antibody specific for the isolated Brachyspira sp. Sask30446 of claim 21.


SEQUENCE LISTING (45 sequences)

SEQ ID NO: 1 - DNA, 23 bp, Artificial sequence
  Synthetic construct
tagcytgcggtatygcwctttgg

SEQ ID NO: 2 - DNA, 23 bp, Artificial Sequence
  Synthetic construct
cttcagaccayccagtagaagcc

SEQ ID NO: 3 - DNA, 23 bp, Brachyspira sp. Sask 30446
tcgctaaattattccaacaagga

SEQ ID NO: 4 - DNA, 21 bp, Brachyspira sp. Sask 30446
aaacgcatttctattccagca

SEQ ID NO: 5 - DNA, 24 bp, Brachyspira sp. Sask 30446
tcatagatgctggaatagaaatgc

SEQ ID NO: 6 - DNA, 22 bp, Brachyspira sp. Sask 30446
gcaccgttaggtaaagtttcca

SEQ ID NO: 7 - DNA, 241 bp, Brachyspira sp. Sask 30446
tcgctaaatttatccaacaaggacaggatatcattaatgaaatagctaaacctgaagtaaaaaaagttatggttgttggtgctggttatataggtgttgagcttatagaagcattcaaaaaccatggtaaagaagttatcttaatggaatctatgcctagagttatggctaactattttgataaagaaatcactgatgaagctgaaaaaagaatcatagatgctggaatagaaatgcgttt

SEQ ID NO: 8 - DNA, 241 bp, Brachyspira sp. Sask 30446
tcgctaaattattccaacaaggacaggatatcattaatgaaatagctaaacctgaagtaaaaaaagttatggttgttggtgctggttatataggtgttgagcttatagaagcattcaaaaaccatggtaaagaagttatcttaatggaatctatgcctagagttatggctaactattttgataaagaaatcactgatgaagctgaaaaaagaatcatagatgctggaatagaaatgcgttt

SEQ ID NO: 9 - DNA, 810 bp, Brachyspira sp. Sask 30446
cctgaaggtttgaaatctgaaggcatcgatgtttatatgggacatgaagtaacaaaaatagactgggctaacaaaaaattacatatcaaagaattaaaaacaggtaaagagtttgaagacaattatgataaacttatacttgctactggttcttggcctgtaactcctcctatagaaggattaaaacaagaaggtactgaatacggtcttaaaaaaggtattttcttcgctaaattattccaacaaggacaggatatcattaatgaaatagctaaacctgaagtaaaaaaagttatggttgttggtgctggttatataggtgttgagcttatagaagcattcaaaaaccatggtaaagaagttatcttaatggaatctatgcctagagttatggctaactattttgataaagaaatcactgatgaagctgaaaaaagaatcatagatgctggaatagaaatgcgtttaggtgaaactgttaaaaagtttgaaggtgatgacagagttaaaaaagttgttaccgataaaggttcttatgatgtagatatggtagttatgtctgttggtttcagacctaatagcgaactttataaagactatttggaaactttacctaacggtgctattgtagtagatactactatgagatcaagcaaagatcctgatgtttatgctataggagactgtgctactgtatattcaagagcttctgaaaaacaagagtatattgctttagctactaatgctgtaagaatgggtattgttgctgctaataatgctttaggaaaacatgttgaatactgcggtact

SEQ ID NO: 10 - DNA, 809 bp, Brachyspira innocens
cctgaaagtttaaaagctgaaggcatagatgtttatatgggacatgatgtaacaaaaatagactgggctaataaaaaattacatgttaaagaattaaaaacaggtaaagagtttgatgataattatgacaaacttattcttgctacaggttcttggcctgtaactcctcctatagaaggtttaatgcaggaaggtactgaatacggacttaaaaaaggtattttcttctctaaattattccagcaaggacaagaaattattgatgaaatagctaaacctgaagtaaaaaaagttatggtagttggtgctggttatataggtgttgaacttatagaagctttcaaaaatcatggtaaagaagttatattaatggaagctatgcctagagttatggctaactattttgacaaagaaattactgatgaagctgaaaaaagaatcaaagaagctggcatagaaatgcatttaggtgaaactgttaaaaaatttgaaggtgatgacagagttaaaaaagttattactgacaaaggttcttatgatgtagatatggtagttatgtctgttggtttcagacctaatagtgagctttataaagattatttagaaactttacctaatggtgctataaaagtagacactactatgaaaactacaaaagatcctaatgtatttgctataggagactgtgctactgtatattcaagagcttctggaaaagaagaatacatcgctttagctactaatgctgtaagaatgggattgttgctgctaacaatgctttaggaaaacatgttgaatactgcggtact

SEQ ID NO: 11 - protein, 79 aa, Brachyspira sp. Sask 30446
AKFIQQGQDIINEIAKPEVKKVMVVGAGYIGVELIEAFKNHGKEVILMESMPRVMANYFDKEITDEAEKRIIDAGIEMR

SEQ ID NO: 12 - protein, 270 aa, Brachyspira sp. Sask 30446
PEGLKSEGIDVYMGHEVTKIDWANKKLHIKELKTGKEFEDNYDKLILATGSWPVTPPIEGLKQEGTEYGLKKGIFFAKLFQQGQDIINEIAKPEVKKVMVVGAGYIGVELIEAFKNHGKEVILMESMPRVMANYFDKEITDEAEKRIIDAGIEMRLGETVKKFEGDDRVKKVVTDKGSYDVDMVVMSVGFRPNSELYKDYLETLPNGAIVVDTTMRSSKDPDVYAIGDCATVYSRASEKQEYIALATNAVRMGIVAANNALGKHVEYCGT

SEQ ID NO: 13 - protein, 270 aa, Brachyspira murdochii
PESLKAEGIDVYMGHDVTKIDWANKKLHVKELKTGKEFDDNYDKLILATGSWPVTPPIEGLMQEGTEYGLKKGIFFSKLFQQGQEIIDEIAKPEVKKVMVVGAGYIGVELIEAFKNHGKEVILMEAMPRVMANYFDKEITDEAEKRIKEAGIEMHLGETVKKFEGDDRVKRVVTDKGSYDVDMVVMSVGFRPNSELYKDYLETLPNGAIKVDTTMKTTKDPNVFAIGDCATVYSRASGKEEYIALATNAVRMGIVAANNALGKHVEYCGT

SEQ ID NO: 14 - protein, 79 aa, Brachyspira pilosicoli
SKLYQQGQDIINEIAKPEVKKVMVVGAGYIGVELIEAFKNHGKEVILMEAMPRVMANYFDKEITDEAEKRIKDAGIELH

SEQ ID NO: 15 - DNA, 21 bp, Artificial Sequence
  Synthetic construct
gatgcttcaggcggagttatg

SEQ ID NO: 16 - DNA, 23 bp, Artificial Sequence
  Synthetic construct
ccacactcatagcataaatactg

SEQ ID NO: 17 - DNA, 21 bp, Artificial Sequence
  Synthetic construct
aggctgggtagaacataatgc

SEQ ID NO: 18 - DNA, 23 bp, Artificial Sequence
  Synthetic construct
tctttactttgataagcaatagc

SEQ ID NO: 19 - DNA, 22 bp, Artificial Sequence
  Synthetic construct
gttggtactaacagaatgaata

SEQ ID NO: 20 - DNA, 20 bp, Artificial Sequence
  Synthetic construct
ccgtctttatcgcgtacatt

SEQ ID NO: 21 - DNA, 22 bp, Artificial Sequence
  Synthetic construct
tgtgttatacaatcagcacttc

SEQ ID NO: 22 - DNA, 22 bp, Artificial Sequence
  Synthetic construct
gtagtaagtattctagctccag

SEQ ID NO: 23 - DNA, 50 bp, Artificial Sequence
  Synthetic construct
cgccagggttttcccagtcacgacgannnngcnggngayggnacnacnac

SEQ ID NO: 24 - DNA, 50 bp, Artificial Sequence
  Synthetic construct
agcggataacaatttcacacaggayknykntcnccraanccnggngcytt

SEQ ID NO: 25 - DNA, 555 bp, Brachyspira sp. Sask 30446
gctactgttttagctcaggctatggttaaagaaggtttgaaaaatgtaactagcggtgctaatcctatgcttattaaaagaggtatagaaaaagctgttagcgaaatagttgcttatatcaaatctgaagctaaacaaattaaaggcaaagaagaaattgctcaggttgctactatttctgctaacaatgataaagaaattggtgctttaatcagtgatgctatggaaaaagttggtaaagaaggcgttatcactgtagaagaagctaaatctttagaaactagtctttctttggtagaaggtatgcagtttgacagaggttatatctctccatatttcgtaactaacggagatagtatgactgctgaattagaagatgctttacttcttatctatgataaaaaaatctctaacatgaaagaacttcttcctatacttgaaaaaattgctcagactggaagacctttcattattatcgctgaagatattgaaaatgaagctttggctactttggtacttaacaaaatgagaggagtattaaatgtatgtgctgtt

SEQ ID NO: 26 - protein, 185 aa, Brachyspira sp. Sask 30446
ATVLAQAMVKEGLKNVTSGANPMLIKRGIEKAVSEIVAYIKSEAKQIKGKEEIAQVATISANNDKEIGALISDAMEKVGKEGVITVEEAKSLETSLSLVEGMQFDRGYISPYFVTNGDSMTAELEDALLLIYDKKISNMKELLPILEKIAQTGRPFIIIAEDIENEALATLVLNKMRGVLNVCAV

SEQ ID NO: 27 - DNA, 581 bp, Brachyspira sp. Sask 30446
aaatatttagaagagagaaatataggttttgatgttggagttacaaaggttcctttggtttgtcagtcttgtatatttgatttgcgtgttggggattataaagttcgtccggatattaatatggcttatgaggcttgtatcaatgctcaaaataataatccaaaaatgggaaattatggagctggtacaggtgctagtgttggaaaaatacttggtgctgattatactatgaagtctgggcttggtttttatgctgttcaaattgatgatgttaaggttggtgctatagttgctcttaatgctttcggcgatatttatgattatgataatggaaaaatgattgctggtcttcttaatgaaaataaagatgggtttagaagttctgaagaggagcttataaaaataacgcagaataataatttgtcttttacttctaataataatatagttacaaatactacaataggagctgtaattacaaatgccaagtttacaaagtcacaaatgggaaaaatagcttctatggcacataacggctttgctagggctataaaaccagtgcatactacagtagatggcga

SEQ ID NO: 28 - protein, 193 aa, Brachyspira sp. Sask 30446
KYLEERNIGFDVGVTKVPLVCQSCIFDLRVGDYKVRPDINMAYEACINAQNNNPKMGNYGAGTGASVGKILGADYTMKSGLGFYAVQIDDVKVGAIVALNAFGDIYDYDNGKMIAGLLNENKDGFRSSEEELIKITQNNNLSFTSNNNIVTNTTIGAVITNAKFTKSQMGKIASMAHNGFARAIKPVHTTVDG

SEQ ID NO: 29 - DNA, 930 bp, Brachyspira sp. Sask 30446
ggagtttgcaggagctatacagattgctggagttaagcctgaagagatagctgctattggtattacaaatcaaagggaaactactgttgtatgggataaaaatactggagagcctatttataatgctatagtatggcaatgtagaagaactgctcctatttgcgatgatttaaagaaaaaaggacttgatacttatataagagaaaatacaggtttagttgttgatgcttatttttctggtacaaaaataaaatggatacttgataatgttcctggtgctagagaaaaagccaataaaggagaattactatttggtacaatagacacttggcttgtatggaaacttacaggcggtaaagttcatgttacagactatactaatgcatcaaggactatgatttataatatcaaagatttaaaatgggacgaaaacattttaagagaattagacattcctatgagcatgcttccagaggtaaaagattcttcttgtgtttacggatatgctaatattaacggtaaagaagtacctatatctggaatagcaggagaccagcagtcagcattattcggtcaggctggatttaataagggcgacacaaaaaatacttatggtactggaagtttcattcttatgaatataggagagaattttatattaagtaaaaacggacttattactactataggtataggatataatggaaaaatagaatatgctttggaaggttctgtatttatagctggtgctgttatacaatgggtacgcgatgaattaagacttcttcatgatgcaaaagacactgaatatttcgctactaaagtaaaagatactaacggagtttatttagtacctgcatttgttggtcttggtgctccttattgggatatgtatgctagaggctgtttagtaggtattacaagaggggt

SEQ ID NO: 30 - protein, 310 aa, Brachyspira sp. Sask 30446
EFAGAIQIAGVKPEEIAAIGITNQRETTVVWDKNTGEPIYNAIVWQCRRTAPICDDLKKKGLDTYIRENTGLVVDAYFSGTKIKWILDNVPGAREKANKGELLFGTIDTWLVWKLTGGKVHVTDYTNASRTMIYNIKDLKWDENILRELDIPMSMLPEVKDSSCVYGYANINGKEVPISGIAGDQQSALFGQAGFNKGDTKNTYGTGSFILMNIGENFILSKNGLITTIGIGYNGKIEYALEGSVFIAGAVIQWVRDELRLLHDAKDTEYFATKVKDTNGVYLVPAFVGLGAPYWDMYARGCLVGITRGV

SEQ ID NO: 31 - DNA, 945 bp, Brachyspira sp. Sask 30446
caggtttagctaattatatattaaaacaaggcggaagtgattataaagttgctataggatatgattcaagaaataattctgatgtattttcaaaagctgctgctgagatactttcttcaaacggaataaaagtttatttatatgatgatattcaccctatttcacttctttcttatgctgttagaagtttaggctgtattgcaggaatagttgttactgctagtcataaccctaaggaatataatggatataaagtttattggactgacggagctcaggttataccgcctcatgacaaaaatatcatagatgaagtattaaaagttaagcctgaagaagtaaaaatgggcgactcttcaaaaattactataataggaaaagatattgaagataaatacatgaatgatttgatggtatatttagttaatccagacatcattaaaaaacatcatgatataaaaatagtttataccccaattcatggttctggatacaaaatggttcctatggctttaagaaaggctggttttacaaatttaacaacattagaaggtgctcagcctccagacggaaattttcctactgtagaatctcctaacccagaaaatcctgaagcattgcagatagccgttaataaagctaaagagataggtgcagaacttgttatgggtaccgacccagactgcgacagaatgggatgtgctttgcttactaaagatggaagctatatgtatcttacaggtaatcagataggatccataatggcatactatctcatcacaaacaaaaaaaatattaaaaatccgtacatagtaaaaacaatagtaacaactgaactagctagggctattgctgatgctaataatgttaagatttacgatgtacttacaggttttaaatggattgctgatgttatagaaagagataaagaaggtacatatttat

SEQ ID NO: 32 - protein, 314 aa, Brachyspira sp. Sask 30446
GLANYILKQGGSDYKVAIGYDSRNNSDVFSKAAAEILSSNGIKVYLYDDIHPISLLSYAVRSLGCIAGIVVTASHNPKEYNGYKVYWTDGAQVIPPHDKNIIDEVLKVKPEEVKMGDSSKITIIGKDIEDKYMNDLMVYLVNPDIIKKHHDIKIVYTPIHGSGYKMVPMALRKAGFTNLTTLEGAQPPDGNFPTVESPNPENPEALQIAVNKAKEIGAELVMGTDPDCDRMGCALLTKDGSYMYLTGNQIGSIMAYYLITNKKNIKNPYIVKTIVTTELARAIADANNVKIYDVLTGFKWIADVIERDKEGTYL

SEQ ID NO: 33 - DNA, 821 bp, Brachyspira sp. Sask 30446
tattaatgccggaatacctgtagacaaaccggcattaactataaatatattatgcggttcaggattaagagctgtatcaatggcagcacaaatgattaaatcaggagatgctgatatagtagttgcaggcggtactgaaaatatgagtatggctccatatacttcttcagctatgagaatgggtgctagaatgggcgaaagtaaaatgcaggatactttacttaatgatgctcttatttgtgcttttgagcattatcatatgggagttactgctgaaaatgttgcagaacaatggggtattacaagacaagaacaagatgagtttgcatgcagaagccagaacagagcagaagaagcagtaaaaagcggaagatttaaagatgaaatagtacctgttacaatcaaaactagaaaaggcgaaatagtagttgatacagatgaacaccctacttttggtactactatggaatctttagctaaattaaaacctgcattcaaaaaagatggtactgttactgctggtaatgcttctggtatcaatgatgctgcttcagctgtagttattatgtctaaagaaaaagctgatgagcttggaattaaacctatggctaagattttaggttatgctactcatggtgttgagcctagaataatgggtataggacctatagaggcaactagaaaggctttgaaaatggctaatcttacagttgaagatatggacttaatagaaagtaatgaggctttcgctgctcagtctatagctgttgcccgtgagttaaaattcaatatggacatagttaatgtcaatggcggagcta

SEQ ID NO: 34 - protein, 273 aa, Brachyspira sp. Sask 30446
INAGIPVDKPALTINILCGSGLRAVSMAAQMIKSGDADIVVAGGTENMSMAPYTSSAMRMGARMGESKMQDTLLNDALICAFEHYHMGVTAENVAEQWGITRQEQDEFACRSQNRAEEAVKSGRFKDEIVPVTIKTRKGEIVVDTDEHPTFGTTMESLAKLKPAFKKDGTVTAGNASGINDAASAVVIMSKEKADELGIKPMAKILGYATHGVEPRIMGIGPIEATRKALKMANLTVEDMDLIESNEAFAAQSIAVARELKFNMDIVNVNGGA

SEQ ID NO: 35 - DNA, 19 bp, Artificial Sequence
  Synthetic construct
gagtttgatcctggctcag

SEQ ID NO: 36 - DNA, 18 bp, Artificial Sequecne
  Synthetic construct
gwattaccgcggckgctg

SEQ ID NO: 37 - DNA, 464 bp, Brachyspira sp. Sask 30446
agcgaacgttggcgatgcgtcttaagcatgcaagtcgagcggacttattcgggcaactggataagttagcggcgaactggtgagtaacacgtaggtaatctgccgtagagtgggggataacccatggaaacatggactaataccgcatatactcttactacacaagtagagtagaggaaaggagcaatccgctttacgatgagcctgcggcctattagcctgttggtggggtaacggcctaccaaagctacgataggtagccgacctgagagggtgaccggccacattgggactgagatacggcccagactcctacgggaggcagcagctgagaatcttccacaatggacgaaagtctgatggagcgacatcgcgtgagggatgaaggccttcgggttgtaaacctcggaaattatcgaagaatgagtgacagtagataatgtaagcctcggctaactacgtgc

SEQ ID NO: 38 - DNA, 24 bp, B. hyodysenteriae
agtgaaatagttgctcatatcaaa

SEQ ID NO: 39 - DNA, 21 bp, B. hyodysenteriae
gcatcactgattaaagaacca

SEQ ID NO: 40 - DNA, 24 bp, B. pilosicoli
acaatgataaagagataggtgctt

SEQ ID NO: 41 - DNA, 26 bp, B. pilosicoli
ctaatgaaaggctagtttctaatgat

SEQ ID NO: 42 - protein, 240 aa, Brachyspira sp. Sask 30446
MRLDEYVHSKGYTESRSKAQDIILAGCVFVNGVKVTSKAHKIKDTDNIEVIQNIKYVSRAGQKLEKAFTEFGISVENKTCLDIGASTGGFTDCLLKHGAKKVYALDVGHNQLVYKLRNDNRVISIEDFNAKDIKREMFNDEIPSIVVSDVSFISISKIAPIIFKELNNLEFWVTLIKPQFEAEKGEVSKGGIIRDDLLREKILNNAISRITEIGFKEVNRTVSPIKGAKGNIEYLAHFII

SEQ ID NO: 43 - protein, 444 aa, Brachyspira sp. Sask 30446
MLSALFSGSETAYTSIDDVTLMRLVREKKIKEEDKKYWEKSSSMIPTLLVGNNIVNISASSIITVFAVRLADILPNISTNLMVTISTATITILIIIFGEILPKVIMRVNAEKMMPYLLYFMKFCHFIFKPITFLMDKITTFIMNYFVPKRLRDAEKRSALSSMDDITTIIHLGHKEGIIKEYTHEMLTGVIDFRNKTVEEIMTPRVDMVCIEAETDVNEIIKLTVETGLSRFPVYEETVDHIIGIFHTRALFKEYVKGGGKLNKIKKKAIDYIMLPYFVPETKTISSLFNDMQKKKLQMVITIDEYGGTAGLVTMEDIIEEIMGDIEDESDKKEADVIRFKGKRIIINGNAPIEDVNKTLKLQLEHEEYQTIAGYVIDMLDHIPEINERFILKGYRVRIMKVEDRRIVEMEFTPLKYTRTNENENTDTQETSDLEKNDLEILNE

SEQ ID NO: 44 - protein, 828 aa, Brachyspira sp. Sask 30446
MFQFHLTSKAKKVIELYAQEEAKRLNHDMVTPEHILLGLLHESEALATRVLLRLKIDLDRLKLELESAMVKSSTTKVFGTLPTAPRVQKLISRSAEEARALSHNYIGTEHLLLGLLREESGTAYNVLTSMGLELTILRQEILKMLGVAGSNMSSMEQTNQEDTIKKVKTPTLDQFARDLTKMARDKALDKVIGRENEVMRVVQILSRRKKNNPILLGEPGVGKTAIVEGLAEKIVAGDVPDILLKKRVLTLDLSSVVAGTKYRGEFEERIKNIVLEIKKANNIIIFIDELHTLIGAGGAEGALDAANMLKPALSRGEIQCIGATTINEYKKYIEKDGALVRRFQPINVEEPSVEDTIEILNGIKPKYEEHHKVKYTDEAITAAAVLSKRYIFERHLPDKAIDLIDEAGSRARLLNMTRPQEFKDLEKKIEELNQNKRNAVDNQNFEDAARIRDEISSLQEELSIKEEKWRGEREKIETFIEEDDIRHVISEITNIPIKRLLNSESKRLIGMEEELHQKVVGQEEAISSISKAIRRSRAGLKTSKRPLGSFIFLGPTGVGKTALAKVLSEFMFGDSDALIRIDMSEFMEKFAVSRLIGAPPGYVGYEEGGGLTEKVRRKPYSLILFDEIEKAHPDVTNILLQVLEEGQLTDNFGRKVDFSNTIIIITSNLGARDIVKGSSLGFNAVGSEKDANDIKNFALEELKQNFNPEFLNRIDDIIVFHTLSKDNLKDIINIMLKELNDAIKERNIVINLSEEAKNYIIDKGFDKKYGARSLRRAIQKEIEDYVSTEILFGNIEDGDTINVDANDGSLIFSYDKSVRTENKELSKS

SEQ ID NO: 45 - protein, 256 aa, Brachyspira sp. Sask 30446
MPIHKLISKILKKKDNNTDKNNYVNLSSLTEAEREIITNTIELKTKSVREIMVPRVDVVMIPIESSYDRVIKAFNKDRNSRIPVYKDGIDDIVGVLYVKDLIDAEEKTFSLKKILHKPLFVPISISLMELLKNFREKQIHIAMVVDEYGGFSGIVSMEDVLEQIIGDIRDEYDEEDEEIKSNDDGTYLVDARTRIDDFNKYDILPPIPDDEADTVGGFLFSYLGRLPKRNEDIEYNGYSFTVVGKSGNIVTKIRIE